(19) United States
(12) Patent Application Publication
(10) Pub. No.: US 2011/0143999 A1
(43) Pub. Date: Jun. 16, 2011

(54) FOWLICIDINS AND METHODS OF ITS USE

(75) Inventors: Guolong Zhang, Stillwater, OK (US); Yanjing Xiao, Stillwater, OK (US)

(73) Assignee: THE BOARD OF REGENTS FOR OKLAHOMA STATE UNIVERSITY, Stillwater, OK (US)

(21) Appl. No.: 12/714,474

(22) Filed: Feb. 27, 2010

Related U.S. Application Data
(62) Division of application No. 11/566,086, filed on Dec. 1, 2006, now abandoned.
(60) Provisional application No. 60/741,989, filed on Dec. 2, 2005.

(51) Int. Cl. A61K 38/16; C07K 14/195; A61P 31/04; A01P 1/00

(52) U.S. Cl. 514/2.4; 530/326

(57) ABSTRACT

Antimicrobial peptides and methods for their use are provided. The peptides are optimized, truncated versions of chicken cathelicidins (“fowlicidins”).

### CROSS-REFERENCE TO RELATED APPLICATIONS

This application is a divisional patent application of U.S. patent application Ser. No. 11/566,086, filed Dec. 1, 2006, which claims the benefit of U.S. Provisional Patent Application 60/741,989, filed Dec. 2, 2005. The complete contents of both of these applications are hereby incorporated by reference.

### SEQUENCE LISTING

This application includes as the Sequence Listing the complete contents of the accompanying text file “Sequence.txt”, created Dec. 1, 2006, containing 32,607 bytes, hereby incorporated by reference.

### BACKGROUND OF THE INVENTION

1. Field of the Invention

The invention generally relates to antimicrobial peptides. In particular, the invention provides optimized truncated versions of chicken cathelicidins (“fowlicidins”).

2. BACKGROUND OF THE INVENTION

The rapid emergence of antibiotic-resistant pathogens poses increasingly serious health concerns worldwide. Approximately 70% of bacteria that cause infections in hospitals are resistant to at least one of the antibiotics most commonly used to treat infections. This antibiotic resistance is driving up health care costs, increasing the severity of disease, and increasing the death rates from certain infections. The annual cost of treating antibiotic-resistant infections in the U.S. is estimated to be as high as $30 billion. Sepsis is another serious medical condition resulting from severe inflammatory response to systemic bacterial infections. According to the CDC, sepsis is the leading cause of death among non-coronary patients in intensive care units and the tenth most common cause of death overall (Martin, G. S., et al. New England Journal of Medicine 2003, 348: 1546).

Antimicrobial peptides (also known as natural antibiotics) comprise a large group of molecules that are capable of killing a broad spectrum of pathogens with similar activities against both antibiotic-susceptible and -resistant bacterial strains and extremely low risks of developing resistance (Zasloff, M. Nature 2002, 415:389). More desirably, many have capacity to bind bacterial endotoxin and neutralize bacterium-induced inflammatory response. Because of the dual capability to kill bacteria and neutralize endotoxins, these antimicrobial peptides hold great promise as a new class of antimicrobial and anti-sepsis agents (Finlay, B. B., and R. E. Hancock. Nature Reviews Microbiology 2004, 2: 497).

Lynn et al., (Immunogenetic 2004; 56: 170-177) describe the bioinformatic discovery and tissue expression pattern of nine novel antimicrobial peptide genes in the chicken, including a cathelicidin-like sequence. However, a structure-function analysis of the peptides is not provided, and no testing or optimization of the amino acid sequences described therein is provided.

Van Kijk et al., (Veterinary Immunology and Immunopathology 106 (2005): 321-327) disclose a novel chicken cathelicidin-like antimicrobial protein. However, a detailed structure-function analysis of the protein is not provided, and no testing or optimization of the protein sequence was undertaken.

There remains an ongoing need to discover new antibiotic agents that are bactericidal and yet display low levels of toxicity to mammalian cells.

### SUMMARY OF THE INVENTION

The present invention is based on the discovery of antibiotic cathelicidins from chickens (“fowlicidins”), and elucidation of the structural and functional relationships of their amino acid sequences to their bactericidal properties. Accordingly, the invention provides novel peptides whose primary sequence is based on or derived from that of the fowlicidins, but optimized with respect to antibiotic and toxicity properties. In general, the optimized peptides are truncated versions of the full length fowlicidins. The optimized, truncated peptides retain excellent bactericidal properties but display reduced toxicity to mammalian cells. In some cases, conservative amino acid substitutions are introduced into the sequences of the peptides.

It is an object of this invention to provide a peptide having amino acid sequence X₀-X₁-P-L-V-X₂ I-X₃-T-V-X₄-A-X₆-X₇-N-L-X₈-X₉-A-I-X₁₀-X₁₁-X₁₂, where X₀=the tripeptide KRF or is absent; X₁=W or L; X₂=the tripeptide PVA or is absent; X₃=R or N; X₄=I or A; X₅=G or P; X₆ G or is absent; X₇=Y, L or I; X₈=Y or L; X₉=R or K; X₁₀=R, K or absent; X₁₁=R, K or absent; and X₁₂=R, K or absent. In one embodiment of the invention, the amino acid sequence of the peptide is selected from the group consisting of

(SEQ ID NO: 9)

W-P-L-V-I-R-T-V-I-A-G-Y-N-L-Y-R-A-I-K-K-K;

(SEQ ID NO: 66)

W-P-L-V-P-V-A-I-N-T-V-A-A-G-I-N-L-Y-K-A-I-K-K-K;

(SEQ ID NO: 11)

W-P-L-V-I-R-T-V-I-A-G-L-N-L-Y-R-A-I-K-K-K;

(SEQ ID NO: 12)

W-P-L-V-I-R-T-V-I-A-G-L-N-L-L-R-A-I-K-K-K;

(SEQ ID NO: 13)

W-P-L-V-I-R-T-V-I-A-G-L-N-L-L-R-A-I-K-K-K;

(SEQ ID NO: 14)

W-P-L-V-I-R-T-V-I-A-G-G-Y-N-L-Y-R-A-I-K-K-K;

(SEQ ID NO: 16)

W-P-L-V-I-R-T-V-I-A-P-Y-N-L-Y-R-A-I-K-K-K;
and

(SEQ ID NO: 18)

K-R-F-W-P-L-V-P-V-A-I-N-T-V-A-A-G-I-N-L-Y-K-A-I-
R-R-K.

In one embodiment of the invention, the carboxyterminus of the peptide is amidated.

In another aspect, the present invention provides a method of treating a bacterial infection in a patient in need thereof. The method comprises the step of administering to the patient a peptide in a quantity sufficient to treat the bacterial infection, the peptide having amino acid sequence: X₀-X₁-P-L-V-X₂-I-X₃-T-V-X₄-A-X₅-X₆-X₇-N-L-X₈-X₉-A-I-X₁₀-X₁₁-X₁₂, where X₀=the tripeptide KRF or is absent; X₁=W or L; X₂=the tripeptide PVA or is absent; X₃=R or N; X₄=I or A; X₅=G or P; X₆=G or is absent; X₇=Y, L or I; X₈=Y or L; X₉=R or K; X₁₀=R, K or absent; X₁₁=R, K or absent; and X₁₂=R, K or absent. In one embodiment of the method, the amino acid sequence of the peptide is selected from the group consisting of

(SEQ ID NO: 9)

W-P-L-V-I-R-T-V-I-A-G-Y-N-L-Y-R-A-I-K-K-K;

(SEQ ID NO: 66)

W-P-L-V-P-V-A-I-N-T-V-A-A-G-I-N-L-Y-K-A-I-K-K-K;

(SEQ ID NO: 11)

W-P-L-V-I-R-T-V-I-A-G-L-N-L-Y-R-A-I-K-K-K;

(SEQ ID NO: 12)

W-P-L-V-I-R-T-V-I-A-G-L-N-L-L-R-A-I-K-K-K;

(SEQ ID NO: 13)

W-P-L-V-I-R-T-V-I-A-G-L-N-L-L-R-A-I-K-K-K;

(SEQ ID NO: 14)

W-P-L-V-I-R-T-V-I-A-G-G-Y-N-L-Y-R-A-I-K-K-K;

(SEQ ID NO: 16)

W-P-L-V-I-R-T-V-I-A-P-Y-N-L-Y-R-A-I-K-K-K;
and

(SEQ ID NO: 18)

K-R-F-W-P-L-V-P-V-A-I-N-T-V-A-A-G-I-N-L-Y-K-A-I-
R-R-K.

In yet another embodiment of the invention, the carboxyterminus of the peptide is amidated. In one embodiment of the invention, the bacterial infection is caused by antibiotic resistant bacteria.

In yet another aspect, the invention provides a method of killing or damaging a bacterium. The method comprises the step of contacting said bacterium with a peptide in a quantity sufficient to kill or damage said bacterium, the peptide having amino acid sequence: X₀-X₁-P-L-V-X₂-I-X₃-T-V-X₄-A-X₅-X₆-X₇-N-L-X₈-X₉-A-I-X₁₀-X₁₁-X₁₂, where X₀=the tripeptide KRF or is absent; X₁=W or L; X₂=the tripeptide PVA or is absent; X₃=R or N; X₄=I or A; X₅=G or P; X₆=G or is absent; X₇=Y, L or I; X₈=Y or L; X₉=R or K; X₁₀=R, K or absent; X₁₁=R, K or absent; and X₁₂=R, K or absent. In one embodiment of the invention, the amino acid sequence of the peptide is selected from the group consisting of

(SEQ ID NO: 9)

W-P-L-V-I-R-T-V-I-A-G-Y-N-L-Y-R-A-I-K-K-K;

(SEQ ID NO: 66)

W-P-L-V-P-V-A-I-N-T-V-A-A-G-I-N-L-Y-K-A-I-K-K-K;

(SEQ ID NO: 11)

W-P-L-V-I-R-T-V-I-A-G-L-N-L-Y-R-A-I-K-K-K;

(SEQ ID NO: 12)

W-P-L-V-I-R-T-V-I-A-G-L-N-L-L-R-A-I-K-K-K;

(SEQ ID NO: 13)

W-P-L-V-I-R-T-V-I-A-G-L-N-L-L-R-A-I-K-K-K;

(SEQ ID NO: 14)

W-P-L-V-I-R-T-V-I-A-G-G-Y-N-L-Y-R-A-I-K-K-K;

(SEQ ID NO: 16)

W-P-L-V-I-R-T-V-I-A-P-Y-N-L-Y-R-A-I-K-K-K;
and

(SEQ ID NO: 18)

K-R-F-W-P-L-V-P-V-A-I-N-T-V-A-A-G-I-N-L-Y-K-A-I-
R-R-K.

In one embodiment of the invention, the carboxyterminus of the peptide is amidated. In yet another embodiment of the invention, the bacterial infection is caused by antibiotic resistant bacteria.

The invention further provides a peptide having amino acid sequence

(SEQ ID NO: 43)

L-V-Q-R-G-R-F-G-R-F-L-R-K-I-R-R-F-R
or

(SEQ ID NO: 44)

R-R-F-R-P-K-V-T-I-T-I-Q-G-S-A-R-F.

In yet another embodiment of the invention, the carboxyterminus of the peptide is amidated.

The invention further provides a method of treating a bacterial infection in a patient in need thereof. The method comprises the step of administering to the patient a peptide in a quantity sufficient to treat the bacterial infection, the peptide having amino acid sequence

(SEQ ID NO: 43)

L-V-Q-R-G-R-F-G-R-F-L-R-K-I-R-R-F-R
or

(SEQ ID NO: 44)

R-R-F-R-P-K-V-T-I-T-I-Q-G-S-A-R-F.

In one embodiment of the invention, the carboxyterminus of the peptide is amidated. In yet another embodiment of the invention, the bacterial infection is caused by antibiotic resistant bacteria.

The present invention also provides a method of killing or damaging a bacterium. The method comprises the step of contacting the bacterium with a peptide in a quantity sufficient to kill or damage the bacterium, the peptide having amino acid sequence

(SEQ ID NO: 43)

L-V-Q-R-G-R-F-G-R-F-L-R-K-I-R-R-F-R
or

(SEQ ID NO: 44)

R-R-F-R-P-K-V-T-I-T-I-Q-G-S-A-R-F.

In one embodiment of the invention, the carboxyterminus of the peptide is amidated. In yet another embodiment of the invention, the bacterial infection is caused by antibiotic resistant bacteria.

### BRIEF DESCRIPTION OF THE DRAWINGS

FIG. 1. Multiple sequence alignment of chicken fowlicidins with representative mammalian cathelicidins. Fowlicidins are aligned with classic cathelicidins (human LL37, mouse CRAMP, porcine Protegrin-1, and bovine Indolicidin) and a group of distantly related neutrophilic granule proteins (NGP) in the pig (pNGP), cow (bNGP), rat (rNGP), and rabbit (P15). Dashes are inserted to optimize the alignment and conserved residues are shaded. The positions of four exon boundaries are indicated by vertical lines. Two intramolecular disulfide bonds in the cathelin pro-sequence are shown. Also indicated are the net positive charge (in parenthesis) and length of each mature cathelicidin as underlined. Because NGP proteins may not be cleaved following activation, no mature sequences are postulated. Note that chicken fowlicidins share higher sequence homology with NGP proteins in the first three exons than with classic mammalian cathelicidins. The SEQ ID NOS. for the peptides are given in Table X.

FIGS. 2A and B. Genomic organization of the chicken fowlicidin cluster (A) and comparative analysis of the mammalian cathelicidin gene clusters (B). The continuous genomic contig containing chicken fowlicidins was obtained by annotation of three whole-genome shotgun sequences (AADN01005055, AADN01005056, and AADN01081708) and additional genomic sequences obtained by PCR and vectorette PCR (see “Experimental Procedures”). The direction of transcription of each gene is indicated by the arrow. In Panel A, the chicken cathelicidin cluster consists of three fowlicidin genes each containing four exons (E) shown as solid rectangles. Located between fowlicidin-1 and fowlicidin-2 genes is chicken CamKV gene, which is homologous to vesicle-associated, calmodulin kinase-like kinase (NP_{—}076951). In Panel B, relative position of each gene is indicated by a solid rectangle or vertical line. Note that chicken fowlicidins, similar to neutrophilic granule proteins (NGP), are located closely adjacent to an evolutionarily conserved gene (KLHL18), but a NGP-like protein is missing in the dog or human genome.

FIG. 3. Phylogenetic analysis of cathelicidins. The tree was constructed by the neighbor-joining method based on the proportion difference (p-distance) of aligned amino acid sites of the full-length peptide sequences. A total of 1000 bootstrap replicates were used to test the reliability of each branch. Numbers on the branches indicate the percentage of 1000 bootstrap samples supporting the branch. Only branches supported by a bootstrap value of at least 50% are shown. The tree constructed with the peptide sequences from the first three conserved exons is essentially the same as the one constructed with the full-length peptides, and therefore, is not shown.

FIG. 4A-C. Antibacterial properties of fowlicidins. E. coli ATCC 25922 was incubated with peptides in 10 mM phosphate buffer, pH 7.4 at 37° C. and surviving bacteria were plated onto trypticase soy agar plates and quantitated as CFU/ml following overnight incubation. A, dose-dependent killing of E. coli by fowlicidins and an ovine cathelicidin (SMAP-29). Bacteria were exposed to indicated peptide concentrations for 2 h followed by quantitative CFU assays. The MIC₅₀ value is indicated as a dotted line. B, Time-dependent killing of E. coli by peptides at MIC₅₀ concentrations. Following exposure to peptides (0.1 μM fowlicidin-1, 0.16 μM fowlicidin-2, and 0.1 μM SMAP-29) or an equal volume of 0.01% acetic acid (control) for 0, 5, 10, 15, 20, 30, and 60 min, surviving bacteria were plated and counted. C, Effect of Salinity on the antibacterial activity of fowlicidins. E. coli were exposed to peptides (0.1 μM fowlicidin-1 and 0.16 μM fowlicidin-2) or an equal volume of solvent (control) in 10 mM phosphate buffer with addition of different concentrations of NaCl (0, 25, 50, 100, and 150 mM). Data shown are means±standard error of the mean (SEM) of 2-4 independent experiments.

FIG. 5A-C. Cytolytic activities of fowlicidins. A, hemolytic activity of fowlicidins and SMAP-29 to chicken erythrocytes. B, hemolytic activity of fowlicidins and SMAP-29 to human erythrocytes. In panels A and B, freshly isolated red blood cells were incubated with different concentrations of peptides in PBS for 2 h before measuring the absorbance at 405 nm for the released hemoglobin. C, cytotoxicity of fowlicidins and SMAP-29 to MDCK cells. Cells were incubated with serially diluted peptides for 24 h in serum-free medium, followed by measurement of the viability of cells by an alamarBlue dye-based, colorimetric method. The EC₅₀ values are indicated as dotted lines. Data shown are means ±SEM of 2-3 independent experiments.

FIG. 6A-D. Neutralization of lipopolysaccharide (LPS) by fowlicidins. A, LPS binding by fowlicidins. Chromogenic Limulus amebocyte lysate assay was used to evaluate the binding of fowlicidins to LPS from E. coli O111:B4. The EC₅₀ value is indicated as a dotted line. Data shown are means±SEM of three independent experiments. B, Hill plot of LPS binding by fowlicidins showing the binding affinity. The plot was graphed from the means in panel A. The Hill's coefficient was derived from the slope of the linear regression. C, Blockage of LPS-induced IL-1β gene expression by fowlicidins. D, Blockage of LPS-induced CCL-2/MCP-1 gene expression by fowlicidins. In panels C and D, RAW264.7 cells were pretreated for 30 min with increasing concentrations (1, 5, and 20 μM) of peptides, and then stimulated with 100 ng/ml LPS or left untreated for 4 h. Total RNA was isolated from cells and subjected to real time RT-PCR. Data shown are a representative of two independent experiments with similar results.

FIG. 7. Solution structure of fowlicidin-1. A ribbon stereo-diagram of the restrained minimized average structure of fowlicidin-1.

FIGS. 8A and B. Helical wheel projections of the central helical regions (residues 6-23) of fowlicidin-1 (A) and its substitution mutant, fowlicidin-1-K⁷L¹²K¹⁴L¹⁶K¹⁸ (B). The representation shows the amphipathic structure of the helical region. Charged residues are indicated in black background, and polar uncharged residues are in gray background. The mutated residues are circled. Notice a significant enhancement in amphipathicity of the mutant peptide relative to the native peptide.

FIGS. 9A and B. LPS-binding isotherms of the deletion (A) and substitution mutants (B) of fowlicidin-1. The EC50 value, indicated by a dotted line in each panel, was defined as the peptide concentration that inhibited LPS-mediated procoagulant activation by 50%. In panel A, ▪ fowlicidin-1(1-26); ◯, fowlicidin-1(8-26); □, fowlicidin-1(1-15); ▴, fowlicidin-1(5-26); □, fowlicidin-1(1-23); , fowlicidin-1(8-26)+fowlicidin-1(1-15). In panel B, ▪ fowlicidin-1(1-26); ▴, fowlicidin-1-L16; and ▾, fowlicidin-1-KLKLK. Data shown are means±SEM of three independent experiments.

FIG. 10. Alignment of representative linear α-helical antimicrobial peptides demonstrating the conservation of a kink induced by glycine near the center. Putatively mature fowlicidin-1 sequence is aligned with representative cathelicidins (mouse CRAMP, rabbit CAP18, bovine BMAP34 and BMAP28 , sheep SMAP34 and SMAP29, and porcine PMAP37) as well as three insect peptides (fruit fly cecropin A1, a putative porcine cecropin P1, and honey bee melittin). Dashes are inserted to optimize the alignment and conserved residues are shaded. Note that each peptide aligned has an α-helix N-terminal to the conserved glycine (boxed) near the center, followed by either a helical or unstructured tail. The only exception is CRAMP, which has a kink at Gly¹¹ instead of Gly¹⁸. The SEQ ID NOS. for the peptides are given in Table X.

FIG. 11. Schematic drawing of the distribution of functional determinants of fowlicidin-1 (SEQ ID NO: 2). Note that the C-terminal helix from Gly¹⁶ to Ile²³ is indispensable for antibacterial, cytolytic, and LPS-binding activities, whereas the three residues (Val⁵-Pro⁷) in the N-terminal unstructured region constitute the core of the second determinant that is critically involved in cytotoxicity and LPS binding, but less significant in the bactericidal activity. The N-terminal helix (Leu⁸-Ala¹⁵) also presumably facilitates the interactions of the C-terminal helix (Gly¹⁶-Ile²³) with lipid membranes.

FIG. 12. Antibacterial activity of fowlicidin-2 and its analogs. Mid-log phase bacteria (5×10⁵ CFU/ml) were incubated overnight with serial twofold dilutions of peptides in the assay medium containing 20% TSB, 25 mM NaHCO₃, and 1 mM NaH₂PO₄ in the presence or absence of 100 mM NaCl. The MIC value of individual peptides against each bacterial strain was determined as the peptide concentration that gave no visible bacterial growth after overnight incubation. The experiments were repeated at least three times and the ranges of MICs were presented.

FIG. 13. Effect of serum on the antibacterial activity of fowlicidin-2 and its analogs. The antibacterial activity was measured by the radial diffusion assay using S. aureus ATCC 25923 and 0.5 μg of each peptide in each well in the presence and absence of 20% or 50% of human serum. The bacterial clearance zones were visualized following overnight incubation at 37° C. The results are representative of two independent experiments.

FIG. 14A-F. Permeabilization of E. coli cytoplasmic membrane of fowlicidin-2 and its analogs. E. coli ML-35p was incubated with each peptide and 1.5 mM of a chromogenic substrate (ONPG) in the presence and absence of 100 mM NaCl. All peptides were used at 0.5 MIC, except for fowlicidin-2(19-31), which was applied at 16 μM. The inner membrane permeability of each peptide was monitored every 2 min for 2 h at 37° C. for the production of o-nitrophenol at 420 nm. Symbols: peptide (-), peptide with 100 mM NaCl ( . . . ) No peptide (. -), and no peptide with 100 mM NaCl ( . . . -). The results are representative of two independent experiments. A, Fowl-2(1-31); B, Fowl-2(1-15); C, Fowl-2(15-31); D, Fowl-2(1-14); E, Fowl-2(1-18); F, Fowl-2(19-31).

FIG. 15A-C. Cytotoxicity of fowlicidin-2 and its analogs. Hemolytic activity was evaluated by incubating individual peptides in serial two-fold dilutions with freshly isolated human erythrocytes in the presence (A) or absence of 10% FBS (B) at 37° C. for 2 h, followed by measuring the released hemoglobin at 405 nm. C, toxicity to human Caco-2 cells. Peptides at 100 μM were incubated with Caco-2 cells at 37° C. for 24 h, and cell viabilities were measured by an Alamar-blue based, colorimetric method. Data shown are means±SEM of 2-3 independent experiments.

FIG. 16A-C. Neutralization of LPS by fowlicidin-2 and its analogs. RAW 264.7 cells were pretreated with or without three different concentrations of peptides (1, 5, and 20 μM) for 30 min, followed by stimulation with 0.1 μg/ml LPS at 37° C. for 4 h. Total RNA was isolated, and the gene expression levels of IL-1β, CCL2/MCP-1, and CCL3/MIP-1a were evaluated by real-time PCR. Data shown are means±SEM of two independent experiments. A, IL-1β; B, CCL-2; C, CCL-3.

FIGS. 17A and B. Tertiary structure of fowlicidin-3 in 50% TFE. A, Ribbon diagram of the minimized average structure of fowlicidin-3. B, Sequence alignment of fowlicidin-1 (SEQ ID NO: 2) and fowlicidin-3 (SEQ ID NO: 18). Dashes are created to maximize the alignment, and the total amino acid residue numbers are also indicated. Vertical bars connecting sequences denote identities, whereas colons mean similarities. The conserved glycine is shaded.

FIG. 18. Permeabilization of bacterial cytoplasmic membrane by fowlicidins. E. coli ML-35p, a lactose permease-deficient strain with constitutive production of β-galactosidase in the cytosol, was diluted to 2.5-5×10⁷ CFU/ml and incubated with 1 μM of fowlicidin-3 or -1 in 10 mM sodium phosphate, pH 7.4, in the presence and absence of 100 mM NaCl at 37° C. A chromogenic substrate for β-galactosidase, p-nitrophenyl-β-D-galactopyranoside (ONPG), was also added to a final concentration of 1.5 mM. The absorbance at 405 nm was monitored every 2 min for the production of p-nitrophenol for up to 1 h. Data shown are representative of two independent experiments with highly similar results.

FIGS. 19A and B. Effect of serum on the antibacterial activity of fowlicidins by radial diffusion assay. A, One μg of fowlicidin-3 and -1, diluted in 0.01% acetic acid with and without 50% human or chicken serum, was added to the wells of the underlay gel containing S. aureus ATCC 25923 (4×10⁵ CFU/ml). After overnight incubation, bacterial clearance zones were recorded, and relative antibacterial activities (%) in the presence of serum were calculated relative to the activities without serum. In panel B, open bars represent no serum controls, whereas striped and solid bars are 50% human and chicken serum, respectively. Data shown are representative of two independent experiments with similar results.

FIGS. 20A and B. Toxicity of fowlicidins to MDCK cells (A) and human erythrocytes (B) in the presence and absence of 10% FBS. 50% effective concentrations (EC₅₀) were indicated as dotted lines in both panels. Data shown are means±SEM of 2-4 independent experiments.

FIGS. 21A and B. Inhibition of LPS-induced expression of IL-1β and CCL3/MIP-1a in RAW264.7 cells. Cells were pretreated for 30 min with and without fowlicidin-3 (0.5, 2.5, and 10 μM) or fowlicidin-1 (2.5 and 10 μM) in duplicate, followed by stimulation for another 4 h with 100 ng/ml LPS. Total RNA was then isolated and subjected to real-time RT-PCR analysis. Data shown are means±SEM of two independent experiments.

FIG. 22. Toxicity of fowlicidin-1 analogs to human Caco-2 epithelial cells.

FIG. 23. Rational design of fowlicidin-1 analogs. The amino acid sequences of exemplary peptides based on fowlicidin-1 are depicted. The SEQ ID NOS. for the peptides are given in Table X.

### DETAILED DESCRIPTION OF THE PREFERRED EMBODIMENTS OF THE INVENTION

The present invention provides novel, optimized antibiotic peptides, the amino acid sequences of which are derived from or based on three newly identified and characterized chicken cathelicidins (“fowlicidins”). The fowlicidins are denominated fowlicidin-1, fowlicidin-2 and fowlicidin-3. The initial identification and characterization of the three chicken fowlicidins is described herein, as is the development of optimized forms of the originally identified, full length peptides. Generally, the optimized versions are truncated forms of the original sequences that display significantly lower toxicity than the parent peptides, and yet retain excellent antibiotic activity. In some cases, amino acid substitutions are also introduced.

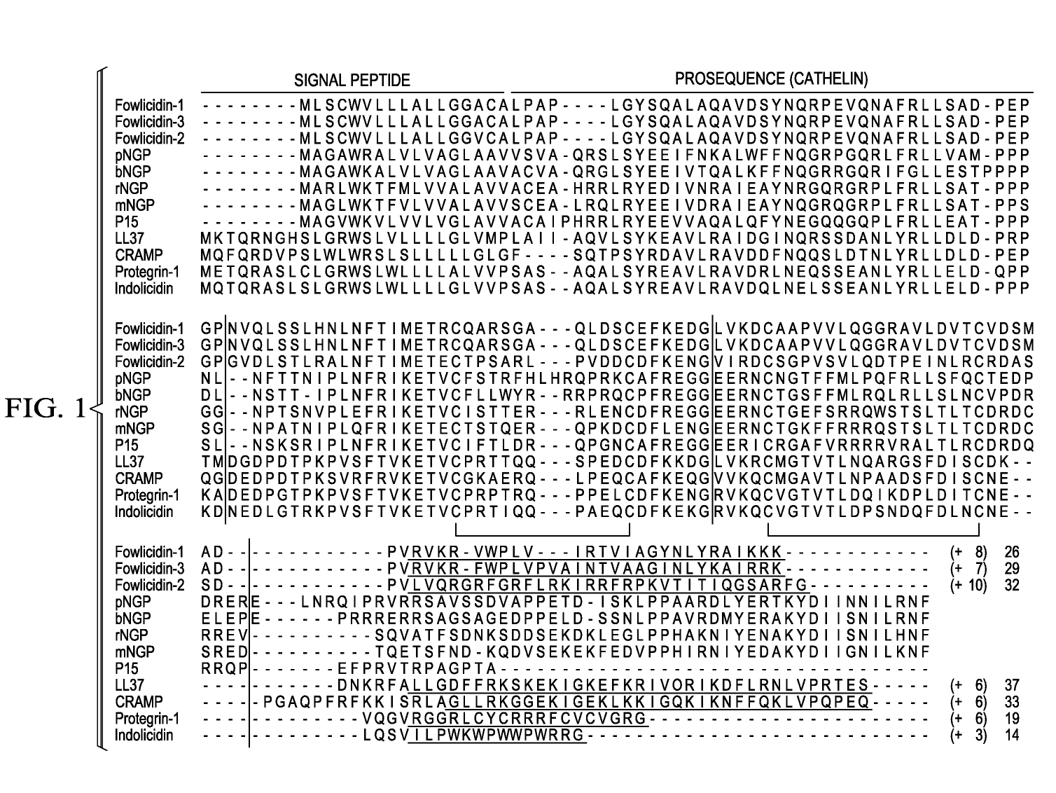

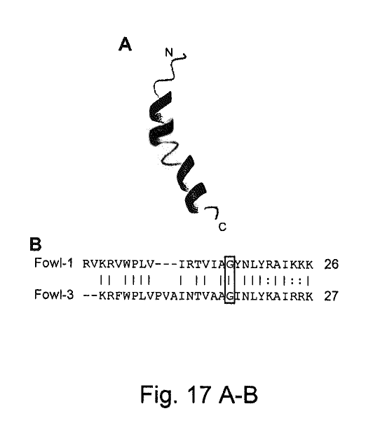

In one embodiment of the invention, the optimized peptides are modified forms of fowlicidin-1 and/or fowlicidin-3, the primary sequences of which are similar (see FIGS. 1 and 17B, and Table X). This category of truncated, optimized peptide has the general formula:

(Formula 1)

X₀-X₁-P-L-V-X₂ I-X₃-T-V-X₄-A-X₅-X₆-X₇-N-L-X₈-X₉-
A-I-X₁₀-X₁₁-X₁₂

where X₀=the tripeptide KRF or is absent (i.e. the sequence may or may not be present at the amino terminus of the peptide); X₁=W or L; X₂=the tripeptide PVA or is absent (i.e. the tripeptide may or may not be present at this position in the peptide); X₃=R or N; X₄=I or A; X₅=G or P; X₆=G or is absent; X₇=Y, L or I; X₈=Y or L; X₉=R or K; X₁₀=R, K or absent; X₁₁=R, K or is absent; and X₁₂=R, K or is absent.

In another embodiment of the invention, the antibiotic peptides are truncated variants of fowlicidin-2 (see Tables VI and X), and have sequences corresponding to amino acids 1-18 of fowlicidin-2 (LVQRGRFGRFLRKIRRFR, SEQ ID NO: 43) or amino acids 15-31 of fowlicidin-2 (RRFRPKVTITIQGSARF, SEQ ID NO: 44), or variants thereof with conservative amino acid substitutions.

In general, the optimized antibiotic peptides of the invention are truncated versions (peptide fragments) of the three original fowlicidins that are identified herein. Those of skill in the art will recognize that, while in some embodiments of the invention, the amino acid sequences of the optimized, truncated antibiotic peptides correspond directly to foreshortened portions or segments of the primary amino acid sequence of the original sequences of the fowlicidin peptides, this need not be the case. The truncated sequences may be further modified. The amino acid sequence of the original (i.e.“natural” or “native” or “parent” or ‘wildtype“) peptide is the amino acid sequence of the fowlicidin as it occurs in nature. Those of skill in the art will recognize that such natural sequences of the three fowlicidins may also display variability, e.g. due to genotypic variations between chickens (for example, between breeds of chickens), or among individual chickens due to spontaneous mutations, etc. All such native sequences of fowlicidins 1-, 2- and 3- as isolated from nature are intended to be comprehended by the present invention, and may serve as the basis for the truncated, optimized peptides of the invention. The sequences of the truncated, optimized peptides of the invention are “based on” or “derived from” or “from” such original sequences. By “based on” or “derived from” or “from” we mean that the sequence of an optimized peptide is the result of modifications of the original, native sequences, e.g. by truncation of the original sequence, by amino acid substitutions, deletions, insertions, or chemical modification.

The amino acid sequence of a truncated, optimized antibiotic peptide as disclosed herein may be altered somewhat from that of a native fowlicidin on which it is based and still be suitable for use in the present invention. While certain specific variations are described, e.g. in Formula 1 where X₁ may be W or L, other variations may also occur to those of skill in the art. For example, certain conservative amino acid substitutions may be made without having a deleterious effect on the ability of the peptide to function as an antibiotic and without increasing toxicity, and in fact may lead to an increase in antibiotic activity and/or a decrease in toxicity. The resulting peptide may be referred to as a “conservative variant”. Those of skill in the art are familiar with the nature of such conservative substitutions, for example, substitution of a positively charged amino acid for another positively charged amino acid; substitution of a negatively charged amino acid for another negatively charged amino acid; substitution of a hydrophobic amino acid for another hydrophobic amino acid; substitution of an aliphatic amino acid for another aliphatic amino acid; substitution of an aliphatic amino acid for another aliphatic amino acid, etc. All such substitutions or alterations of the sequence of the peptides of the invention are intended to be encompassed by the present invention, so long as the resulting peptide is still bactericidal. In general, such substituted sequences will be at least about 50% identical to the corresponding sequence in the native protein, preferably about 60 to 70, or even 70 to 80, or 80 to 90% identical to the wild type sequence, and preferably about 95 to about 100% identical.

In addition, certain other modifications (e.g. chemical modifications) of the optimized peptides of the invention are also contemplated. For example, the carboxyl terminus of the peptides may be amidated; reactive groups may be sufonylated, lipidated, etc.; or L-amino acids of these sequences can be changed to or substituted with D-amino acids.

Further, other variations of the sequences disclosed herein may also be carried out, e.g. the addition of a label or tag to the peptide to facilitate the isolation or detection of the peptides; removal or creation of a protease cleavage site; addition of charged or hydrophilic residues to promote solubility of the peptides; addition of specific residues to promote secondary structural elements (e.g. to modulate helicity, amphipathicity, hydrophobicity, or cationicity); etc.

In addition, the amino acid sequences of the peptides of the invention need not contain the precise number of residues as the exemplary optimized peptides disclosed herein. Certain deletions or additions may be tolerated, so long as the resulting peptide is bactericidal and of sufficiently low toxicity.

Chimeric polypeptides that contain more than one (i.e. multiple) antibiotic peptide sequence within a polypeptide are also envisioned. The multiple peptides may be in tandem within a single, linear polypeptide chain, and may be separated, for example, by spacer peptides, many examples of which are known in the art. Alternatively, the structure of such a chimera may be branched, or a combination of linear and branched. The peptides that make up the chimera may be the same or different.

The optimized peptides of the invention are“antimicrobial” or “antibiotic” or “bactericidal”. By “antibiotic” or “bactericidal” we mean that the peptides exhibit a minimum inhibitory concentration in the low micromolar concentration range (<10 μM) when measured by standard broth microdilution assay as recommended by the Clinical and Laboratory Standards Institute (CLSI). In addition, the peptides are non-toxic or of low toxicity. By “non-toxic”and/or of “low toxicity”, we mean that lysis of 50% of erythrocytes or kiling of 50% mammalian cells occurs at a concentration of >50 μM peptide. Those of skill in the art will recognize that as long as the concentration of peptide that is required in order to be bactericidal is below the level of peptide that is toxic, the peptides may be useful as bactericides.

The antimicrobial peptides of the invention may be used in a variety of ways. As their designation suggests, they may be used as antimicrobial agents, e.g. as bactericidal agents, in order to kill or damage bacteria. Suitable scenarios for such a use of the antimicrobial peptides include but are not limited to: treatment of established bacterial infections (for example in bacterial hosts or potential bacterial hosts such as humans, other mammals, or other living organisms that are susceptible bacterial infections); or prophylactically for the prevention of bacterial infections in such hosts (e.g. the antimicrobial peptides may be administered to individuals whose immune systems are compromised and who may be susceptible bacterial infections); or administered topically to areas of a host that are susceptible to infection, e.g. to areas of the body that are likely sites for bacterial growth, e.g. the gums, open wounds, vaginal and groin area, bed sores or areas which are likely to develop into bedsores, areas which are likely to be moist, e.g. under dressings, diapers, etc.); and the like.

A wide variety of bacterial infections may be treated or prevented by administration of the antimicrobial peptides of the present invention. Examples of such bacteria include but are not limited to: coliform bacteria such as Escherichia coli; Salmonella specis, e.g. S. typhimurium, S. enteritidis, and S. choleraesuis; Klebsiella species, e.g. K. pneumoniae; Pseudomonas species, e.g. P. aeruginosa; Listeria species e.g. L. monocytogenes; Staphylococcus species e.g. S. aureus; Mycobacterium species e.g. M. tuberculosis Enterococcus species, e.g. E. faecalis; Campylobacter species, e.g. C. jejuni, C. coli, and C. fetus; and Clostridium species, e.g. C. perfringens, C. difficile, C. tetani, and C. botulinum In particular, the peptides of the invention may be used to combat bacteria that are resistant to conventional antibiotics, such as MRSA (methicillin-resistant Staphylococcus aureus), VRSA (Vancomycin-resistant S. aureus), VRE (Vancomycin-Resistant Enterococcus), Penicillin-Resistant Enterococcus, Penicillin-resistant Streptococcus pneumoniae, isoniazid/rifampin-resistant Mycobacterium tuberculosis and other antibiotic-resistant strains of E. coli, Salmonella, Campylobacter, and Streptococci. Such bacteria are herein referred to as “antibiotic-resistant” or “drug-resistant” or “multidrug-resistant”, or by other similar terms that are well understood in the art.

While both Gram-negative and Gram-positive bacterial infections may be treated with the peptides of the invention, certain of the optimized peptides are especially active against one or the other category of bacteria, e.g. fowlicidin-1(1-15) is more active against Gram-negative than Gram-positive bacteria. Such selectivity may advantageous when the identity of the bacterial infection being treated is known.

Several diseases or disease conditions that are associated with bacterial infections may be treated with the antibiotic peptides of the invention. Examples of such diseases or conditions include but are not limited to cystic fibrosis-associated chronic respiratory infections, inflammatory bowel diseases (particularly Crohn's disease), acne, and catheter-related infections, and others. In addition, the antibiotic peptides of the invention may be administered prophylactically to patients who are at risk for developing bacterial infections, e.g. those with compromised immune systems due to, for example, HIV infection, chemotherapy, etc.

The present invention also provides new compositions for use in administration to patients (generally humans or mammals). The compounds included one or more of the antimicrobial peptides of the invention. The compositions include one or more substantially purified antimicrobial peptides as described herein, and a pharmacologically suitable carrier. In some embodiments, a single type of antimicrobial peptide may be administered. In other embodiments a “cocktail” or mixture of different antimicrobial peptides may be administered. The preparation of such compositions for use as antimicrobial agents is well known to those of skill in the art. Typically, such compositions are prepared either as liquid solutions or suspensions, however solid forms such as tablets, pills, powders, pastes, ointments, suppositories, and the like are also contemplated. Solid forms suitable for solution in, or suspension in, liquids prior to administration may also be prepared. The preparation may also be emulsified, or incorporated into nanoparticles, micorparticles, biodegradable polymers such as polylactide (PLA) and its copolymers with glycolide (PLGA), etc. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of antimicrobial peptides in the formulations may vary. However, in general, the amount in the formulations will be from about 1-99%, weight/volume.

The antimicrobial peptide compositions (preparations) of the present invention may be administered by any of the many suitable means which are well known to those of skill in the art, including but not limited to by injection, inhalation, orally, intranasally, by ingestion of a food product containing the antimicrobial peptide, topically, as eye drops, via sprays, incorporated into dressings or bandages (e.g. lyophilized forms may be included directly in the dressing), etc. In preferred embodiments, the mode of administration is topical or orally or by injection. In addition, the compositions may be administered in conjunction with other treatment modalities such as substances that boost the immune system, various chemotherapeutic agents, other antibiotic agents, and the like.

The present invention also provides a method of killing or damaging bacteria. The method involves contacting the bacteria with the antimicrobial peptides of the invention. In some instances, the bacteria will be killed outright, and signs or symptoms of bacterial colonization or infection will be completely eradicated. However, those of skill in the art will recognize that much benefit can be derived even if all bacteria in a population are not killed outright. For example, in some cases, the ability of the bacteria to carry out metabolic reactions may be slowed or otherwise attenuated by exposure to the antimicrobial peptides, or the reproductive potential of the bacteria may be decreased. All such lessening of the bacteria's ability to flourish in an environment in which they would typically establish colonies and persist may be of benefit to a host organism in need of treatment with the antimicrobial peptides of the invention.

While in one embodiment of the invention, treatment of bacterial host organisms or potential bacterial host organisms is contemplated (e.g. humans and other mammals, so that veterinary uses are also included), other uses of the antimicrobial peptides of the invention will also occur to those of skill in the art. For example, the treatment of surfaces for food preparation or of edible substances that might otherwise become colonized by bacteria; use in cleansing products such as soaps, detergents, lotions, etc.

The following Examples provide certain exemplary embodiments of the invention and are intended to be illustrative of the invention, but should not be construed as limiting in any way.

### EXAMPLES

### Example 1

### Identification and Characterization of Three Chicken Cathelicidins with Potent Antimicrobial Activity

Cationic antimicrobial peptides comprise a large group of gene-encoded molecules that have been discovered in virtually all species of life, playing a critical role in innate host defense and disease resistance (1-4). Two major families of antimicrobial peptides exist in mammals, namely defensins and cathelicidins. Whereas defensins are characterized by the presence of six cysteines at well-defined positions (5,6), all cathelicidins share a highly conserved “cathelin” pro-sequence at the N-terminus, followed by diversified, cationic mature sequences at the C-terminus (7-9). Cathelicidins are most abundantly present in the granules of phagocytic cells and also to a lesser extent in many other cell types such as mucosal epithelial cells and skin keratinocytes (7-9).

Upon activation, most cathelicidin precursors are proteolytically cleaved to release the cathelin domain and the C-terminal mature peptides with antimicrobial activities, although the unprocessed or differentially processed forms are often found in the biological fluids where cathelicidins are expressed (8,9). The physiological role of the cathelin domain or uncleaved precursors remains elusive, but is more likely to be involved in immune modulation other than just bacterial killing (10,11).

In addition to their ability to directly kill a wide range of bacteria, fungi, and enveloped viruses, mature cathelicidins are actively involved in various phases of host defense. Certain cathelicidins are found to chemoattract and activate a variety of immune cells, inhibit NADPH oxidase, kill activated lymphocytes, and promote angiogenesis and wound healing (1,8,9). Consistent with their critical role in host defense and disease resistance, aberrant expression of cathelicidins are often associated with various disease processes. For example, LL-37/hCAP-18 deficiency correlates with recurrent skin infections in atopic dermatitis patients (12) and chronic periodontal disease in morbus Kostmann patients (13). Similarly, deletion of the cathelicidin gene (CRAMP) in mice resulted in a loss of protection against skin infection by Group A Streptococcus (14) or oral infection with murine enteric pathogen Citrobacter rodentium (15). Conversely, local or systemic administration of cathelicidins conferred enhanced protection against experimental infections (16-20).

A common mechanism by which cathelicidins kill bacteria appears to be mediated through physical interactions with negatively charged microbial membrane phospholipids, followed by membrane disruption (3,21). Many cathelicidins exhibit LPS-binding activity, and the binding affinity is often positively correlated with their antibacterial activity (7). Because of this physical mechanism, these peptide antibiotics are equally effective in killing both drug-resistant and susceptible strains with little possibility of developing resistance (3,7). One side-effect commonly associated with cathelicidins is their cytotoxicity to mammalian cells; however, the concentrations that are required to exert an appreciable degree of cytolytic effect are often much higher than the microbicidal concentrations (7).

To date, cathelicidins have been discovered in a range of mammalian species (8,9). In contrast to the vast majority of “classic” cathelicidins, P15 in rabbits (22) and neutrophilic granule protein (NGP)¹ in mice (23) are distantly related to classic cathelicidins with less homology in the cathelin domain. Hagfish was also found recently to contain two cathelicidin-like sequences (24). However, the evolutionary relationship of these cathelicidins remains uncertain.

Following a genome-wide computational screening and molecular cloning, the complete repertoire of the cathelicidin gene family in the chicken was identified and functionally analyzed. Discovery of these non-mammalian cathelicidins helped reveal for the first time the origin and evolution of mammalian cathelicidins. The data clearly suggested that fowlicidins and mammalian NGPs are likely to originate from a common ancestral gene prior to the separation of birds from mammals and that other classic mammalian cathelicidins may have been duplicated from the NGP gene after the split of mammals and birds. Moreover, a series of functional analyses indicated that these chicken cathelicidins are among the most efficacious cathelicidins that have been identified to date with potent antibacterial and LPS-neutralizing activities, making them attractive candidates as novel antimicrobial and anti-sepsis agents.

### Experimental Procedures

Computational Search for Novel Chicken Cathelicidins—To identify potential novel cathelicidins in the chicken, all known cathelicidin peptide sequences discovered in the hagfish and mammals were individually queried against the translated chicken expressed sequence tags (EST), nonredundant sequences (NR), unfinished high throughput genomic sequences (HTGS), and whole genome shotgun sequences (WGS) in GenBank by using the TBLASTN program (25) as previously described (26-28). All potential hits were then examined for presence of the characteristic cathelin domain, including the highly conserved four cysteines. If necessary, the genomic sequences containing chicken cathelicidin genes were retrieved from GenBank to predict the exon sequence and genomic structure by using GenomeScan (29).
Cloning of the Chicken Fowlicidin Genes—Because no complete genomic sequence is available in GenBank for any of the three fowlicidin genes, the missing sequence of each gene was cloned separately from chicken genomic DNA that was isolated from liver using a genomic DNA isolation kit (Zymo Research, Orange, Calif.). The first exon/intron sequence of the fowlicidin-2 gene that is missing in the WGS sequence (AADN01005055) was cloned by using a genome walking approach, namely vectorette PCR, as previously described (30,31). Briefly, chicken genomic DNA was digested separately with the blunt-end restriction enzymes (DraI, EcoRV, PuvI, RsaI, and StuI), followed by ligation with annealed, bubble oligonucleotides (5′-CAAGGGAGGACGCTGTCTGTCGAAGGTAAGGAACGGACGAGAGAAGGGA GAG-3′ (SEQ ID NO: 46), and 5′-CTCTCCCTTCTCGAATCGTAACCGTTCGTACG AGAATCGCTGTCCTCTCCTTG-3′ (SEQ ID NO: 47). Subsequent PCR was performed by using the forward primer indicated in the underlined region of the bubble oligonucleotide and a gene-specific reverse primer. Nested PCR was further performed with the same forward primer and a second gene-specific reverse primer. Two rounds of vectorette PCR were performed to obtain a total of 1.8 kb upstream sequence of the fowlicidin-2 gene.

Because only a partial sequence of the last exon of the fowlicidin-1 gene is present in AADN01081708, the entire fowlicidin-1 gene sequence was directly cloned from genomic DNA by PCR using the primers

(SEQ ID NO: 48)

(Forward: 5′-GTTTCCGCATTGCCCAACTTCAG-3′;

(SEQ ID NO: 49)

Reverse: 5′-GGAACAGTGCTAACAGTGGCTC-3′,

which are located in the first and last exons flanking the open reading frame, according to the EST Sequence. The missing first intron sequence of the fowlicidin-3 gene, i.e., the gap between AADN01005055 and AADN01005056, was cloned from chicken genomic DNA by PCR using primers (Forward: 5′-GCTGTGGACTCCTACA ACCAAC-3′(SEQ ID NO: 50; Reverse: 5′-TTGAGGTTGTGCA GGGAGCTGA-3′(SEQ ID NO: 51) located in two flanking exons. All PCR products were recovered from agarose gel, ligated into pGEM T-Easy Vector (Promega, USA), and sequenced from both directions.
Assembly of the Chicken Cathelicidin Gene Cluster—To confirm the orientation of three fowlicidin genes on the chromosome, additional PCR reactions were performed to clone the intergenic sequences with chicken genomic DNA and combinations of gene-specific primers located in the first and last exons of each fowlicidin gene. The DNA sequence between fowlicidin-1 and fowlicidin-2 was obtained by using the primers (Forward: 5′-CGCTGGTCATCAGGACTGTGA T-3′ (SEQ ID NO: 52); Reverse: 5′-CCATCGTGTCTCCATTCTA TC-3′ (SEQ ID NO: 53), while the sequence between fowlicidin-2 and fowlicidin-3 was obtained by using the primers (Forward: 5′-CACCGTGTTGATGGCC ACTGG-3 (SEQ ID NO: 54); Reverse: 5′-TGAGGCCACCGAGTG TCACCT-3′SEQ ID NO: 55). PCR products were subsequently cloned into pGEM T-Easy Vector and sequenced from both directions. No other primer combinations yielded any PCR products.

To generate a continuous, gap-free cathelicidin gene cluster, three WGS sequences containing fowlicidin genes (AADN01005055, AADN01005056, and AADN01081708) were retrieved from GenBank and annotated together with our newly cloned intra- and intergenic sequences. Structures of fowlicidin genes were determined by comparing their cDNA sequences with the assembled genomic contig. Chromosomal location of the chicken cathelicidin gene cluster was revealed by using the Map Viewer Program (at the website located at ncbi.nlm.nih.gov/ mapview/) in the most current chicken genome assembly (Build 1.1) released on Jul. 1, 2004.
Alignment and Phylogenetic Analysis of Chicken Cathelicidins—Multiple sequence alignment was constructed by using the ClustalW program (version 1.82) (32). The phylogenetic tree was constructed using the neighbor-joining method (33) by calculating the proportion of amino acid differences (p-distance) among all known cathelicidin precursors with and without the last exon sequence. The reliability of each branch was tested by 1000 bootstrap replications.
Peptide Synthesis—Given that valine is the preferred cleavage site for elastase in the processing and maturation of bovine and porcine cathelicidins (34,35), we reasoned that the first valine in the fourth exon of fowlicidin-1 and -2 (FIG. 1) is likely to be cleaved by chicken elastase. Therefore, putative mature fowlicidin-1 (RVKRVWPLVIRTVIAGYNL YRAIKKK (SEQ ID NO: 2) and -2 (LVQRGRFGRFLRKIRRFRPKVTITIQGSARF (SEQ ID NO: 17) were chemically synthesized by SynPep (Dublin, Calif.), and a sheep cathelicidin, SMAP-29 (RGLR RLGRKIAHGVKKYGPTVLRIIRIA-NH2 (SEQ ID NO: 56) was synthesized by Bio-Synthesis (Lewisville, Tex.) by the standard solid-phase synthesis method. All peptides were purified to >95% purity through reverse phase-HPLC. The mass and purity of each peptide was confirmed by the matrix-assisted laser desorption/ionization time-of-flight (MALDI-TOF) mass spectrometry using the Voyager DE-PRO instrument (Applied Biosystems, Foster City, Calif.) housed in the Recombinant DNA/Protein Core Facility of Oklahoma State University. The molecular masses of three peptides are as follows: fowlicidin-1 (calculated: 3141.9 and observed: 3141.6), fowlicidin-2 (calculated: 3760.6 and observed: 3760.1), and SMAP-29 (calculated: 3199.0 and observed: 3198.7).
Bacterial Culture—Gram-negative bacteria (Escherichia coli ATCC 25922, E. coli O157:H7 ATCC 700728, Salmonella typhimurium ATCC 14028, Klebsiella pneumoniae ATCC 13883, and Pseudomonas aeruginosa ATCC 27853), and Gram-positive bacteria (L. monocytogenes ATCC 19115 and Staphylococcus aureus ATCC 25923) were purchased from either ATCC (Manassas, Va.) or MicroBiologics (St. Cloud, Minn.) and tested individually against fowlicidins and SMAP-29. Three multidrug-resistant bacterial strains (S. typhimurium DT104 ATCC 700408, S. aureus ATCC BAA-39, and S. aureus ATCC 43300) were also purchased from ATCC and used in the antibacterial testing. All bacteria were maintained on trypticase soy agar (TSA) plates. Fresh colonies were cultured and subcultured in trypticase soy broth (TSB) with shaking at 250 rpm at 37° C. in a shaking incubator.
Antibacterial Assays—Standard colony counting assay was used to determine the antibacterial activity of fowlicidins as previously described (36). Briefly, overnight cultures of bacteria were subcultured for additional 3-5 h at 37° C. in TSB to the mid-logarithmic phase, washed once with 10 mM sodium phosphate buffer, pH 7.4, and suspended to 4×10⁵ colony forming units (CFU)/ml in the same buffer. Bacteria (90 μl) were dispensed into 96-well microtiter plates, followed by addition of 10 μl of serial twofold dilutions of peptides in duplicate. After 2-h incubation at 37° C., surviving bacteria were counted (CFU/ml) after serial plating onto TSA plates and overnight incubation. Minimum inhibitory concentration (MIC₉₀) of individual peptides against each bacterial strain was determined as the lowest concentration that reduced bacterial growth by 90%.
   For the kinetics of bacterial killing, fowlicidin-1 (0.1 μM), fowlicidin-2 (0.16 μM), and SMAP-29 (0.1 μM) at MIC₉₀ concentrations were incubated separately with E. coli ATCC 25922 at 37° C. in 10 mM sodium phosphate buffer, pH 7.4. The reaction was stopped by addition of ice-cold PBS at 0, 5, 10, 15, 20, 30, and 60 min and plated immediately for counting viable bacteria. To study the effect of salinity on the antimicrobial activity, fowlicidin-1 (0.1 μM) and fowlicidin-2 (0.16 μM) were incubated separately with E. coli ATCC 25922 for 2 h with different concentration of NaCl (0, 25, 50, 100, and 150 mM) in 10 mM sodium phosphate buffer, pH 7.4. Surviving bacteria were counted, following overnight incubation on TSA plates.

To examine the antibacterial spectrum of each peptide, a modified broth microdilution assay was used essentially as described (37). Briefly, bacteria were subcultured to the mid-log phase, washed with 10 mM sodium phosphate buffer and suspended to 5×10⁵ CFU/ml in 1% cation-adjusted Mueller Hinton broth (MHB) (BBL, Cockeysville, Md.) with and without 100 mM of NaCl. Bacteria (90 μl) were then dispensed into 96-well plates, followed by addition in duplicate of 10 μl of serially diluted peptides in 0.01% acetic acid. Because of poor growth of P. aeruginosa ATCC 27853 in 1% MHB, this strain was grown in 10% cation-adjusted MHB with peptides in the presence and absence of 100 mM NaCl. After overnight incubation at 37° C., the MIC value of each peptide is determined as the lowest concentration that gave no visible bacterial growth.
Hemolysis Assay—The hemolytic activities of fowlicidins were determined essentially as described (38,39). Briefly, fresh human and chicken blood were collected, washed twice with PBS, and diluted to 0.5% in PBS with and without addition of 10% FBS, followed by dispensing 90 μl into 96-well plates. Different concentrations of peptides (10 μl) dissolved in 0.01% acetic acid were added in duplicate to cells and incubated at 37° C. for 2 h. Following centrifugation at 800×g for 10 min, the supernatants were transferred to new 96-well plates and monitored by measuring the absorbance at 405 nm for released hemoglobin. Controls for 0 and 100% hemolysis consisted of cells suspend in PBS only and in 1% Triton X-100, respectively. Percent hemolysis (%) was calculated as [OD_{405 nm, peptide}−OD_{405 nm, PBS})/(OD_{405 nm, 1% Triton X-100}−OD_{405 nm, PBS)}]×100. The effective concentration (EC₅₀) was defined a the peptide concentration that caused 50% lysis of erythrocytes.
Cytotoxicity Assay—The cytotoxic effect of fowlicidins on mammalian cells was measured by using the alamarBlue dye (Biosource), which has been shown to be equivalent to the classic MTT-based assay (40). Madin-Darby canine kidney (MDCK) epithelial cells were purchased from ATCC and maintained in DMEM with 10% FBS. MDCK cells were seeded into 96-well plates at 1.5×10⁵/well. Following overnight growth, the cells were washed once with DMEM, followed by addition of 90 μl of fresh DMEM with or without 10% FBS, together with 10 μl of serially diluted peptides in 0.01% acetic acid in duplicate. After incubation for 18 h, 10 μl of alamarBlue dye was added to cells for 6 h at 37° C. in a humidified 5% CO₂ incubator. The fluorescence of dye was read with excitation at 545 nm and emission at 590 nm. Percent cell death (%) was calculated as [1−(F_{peptide}−F_{background})/(F_{acetic acid}−F_{background})]×100, where F_{peptide} is the fluorescence of cells exposed to different concentrations of peptides, F_{acetic acid} the fluorescence of cells exposed to 0.01% acetic acid only, F_{background} is the background fluorescence of 10% alamarBlue dye in cell culture medium without cells. Cytotoxicity (EC₅₀) of individual peptides was defined as the peptide concentration that caused 50% cell death.
LPS Binding Assay—The binding of LPS to fowlicidins was measured by the kinetic chromogenic Limulus amebocyte lysate assay (Kinetic-QCL 1000 kit; BioWhittaker, Walkersville, Md.) as previously described (37,41). Briefly, 25 μl of serially diluted peptide were added in duplicate into 25 μl of E. coli O111:B4 LPS containing 0.5 endotoxin units/ml and incubated for 30 min at 37° C., followed by incubation with 50 μl of the amoebocyte lysate reagent for 10 min. The absorbance at 405 nm was measured at 10 and 16 min after addition of 100 al of chromogenic substrate, Ac-Ile-Glu-Ala-Arg-ρ-nitroanilide. Percent LPS binding was calculated as {[ΔD1−ΔD2+ΔD3]/ΔD1}×100, where ΔD1 represents the difference in the absorbance between 10 and 16 min for the sample containing LPS only, ΔD2 represents the difference in the absorbance between 10 and 16 min for the samples containing LPS and different concentrations of peptides, and ΔD3 represents the difference in the absorbance between 10 and 16 min for the samples containing different concentrations of peptides with no LPS. Hill plot was graphed as described (37,41) by plotting log₁₀ fowlicidin concentrations against log₁₀ [F₁/(1.0−F₁)], where F₁ was the fractional inhibition of LPS binding activity.
Modulation of LPS-induced Proinflammatory Gene Expression by Fowlicidins—Mouse macrophage RAW 264.7 cells were plated in 12-well plates at 5×10⁵ cells/well in DMEM with 10% FBS and allowed to grow overnight. The cells were pretreated for 30 min with 1, 5, and 20 μM of fowlicidin-1, fowlicidin-2, and SMAP-29 in duplicate, followed by stimulation with 100 ng/ml LPS from E. coli O114:B4 (Sigma) for 4 h. The supernatant was removed and total RNA was isolated from cells using TRIzol (Invitrogen) according to the manufacturer's instructions. The first strand cDNA from 1.5 μg of each RNA sample was synthesized at 42° C. for 30 min by using QuantiTect® Reverse Transcription Kit (Qiagen), which includes removal of genomic DNA contamination prior to cDNA synthesis.

The first-strand cDNA of each sample was then used as a template for subsequent real-time PCR amplification by using QuantiTect® SYBR Green qRT-PCR Kit (Qiagen) and MyiQ® Real-Time PCR Detection System (Bio-Rad). Three common proinflammatory cytokines and chemokines, namely interleukin (IL)-1β, CC chemokine ligand 2 (CCL2)/MCP-1, and CCL3/MIP-1a, were selected. All primers were designed to expand at least an intron sequence (Table I). The PCR reaction was set up in a total volume of 15 μl containing 0.4 μM of each primer and 0.2 μg of the first-strand cDNA. PCR cycling conditions were as follows: 95° C. for 10 min, followed by 50 cycles of 95° C. for 15 sec, 55° C. for 30 sec, and 72° C. for 30 sec.

TABLE I
Primer sequences used for real-time RT-PCR
analysis of murine cytokines and chemokines

| | | | Product | |
|---|---|---|---|---|
| | Primer Sequence | | Size (bp) | |
| Gene | Forward | Reverse | cDNA | |
| IL-1β | AGAATCTATACCTGTC | TGTGCTCTGCTTGTGA | 916 | 195 |
| | CTGTGT | GGTG | | |
| | (SEQ ID NO: 57) | (SEQ ID NO: 58) | | |
| CCL2 | ACAAGAGGATCACCAG | CTGAAGACCTTAGGG | 511 | 186 |
| | CAGC | CAGATG | | |
| | (SEQ ID NO: 59) | (SEQ ID NO: 60) | | |
| CCL3 | CACGCCAATTCATCGT | CATTCAGTTCCAGGT | 372 | 147 |
| | TGAC | CAGTG | | |
| | (SEQ ID NO: 61) | (SEQ ID NO: 62) | | |
| β-actin | GGAGATTACTGCTCTG | CTCCTGCTTGCTGATC | 264 | 139 |
| | GCTC | CACA | | |
| | (SEQ ID NO: 63) | (SEQ ID NO: 64) | | |

Gene expression levels were quantified by the comparative Δ ΔC_{T} method as described (42) by using β-actin as an internal standard for normalization. The ΔC_{T} value was determined by subtracting the C_{T} value of each sample from that of 13-actin in the corresponding sample. The Δ ΔC_{T} values were further calculated by subtracting the highest mean ΔC_{T} value as an arbitrary constant from all other ΔC_{T} values. Relative gene expression levels were calculated using the formula 2^{-ΔΔCt}. The presence of contaminating genomic DNA was determined by including a no-reverse transcriptase control and signal generated by primer dimers was determined through no-template controls. Melting curve analysis (55-95° C.) was performed and confirmed no visible nonspecific amplification of any PCR products from genomic DNA or primer dimers.

### Results

Identification of Three Novel Chicken Cathelicidin Genes—To identify potential cathelicidins in the chicken, all known cathelicidin peptide sequences were queried individually against the translated genomic and EST sequences in GenBank by using the TBLASTN program (25) as previously described (26-28). As a result, seven chicken EST sequences (GenBank accession numbers BX936022, BU106516, AJ393748, CB018183, BU420865, CR389785, and BQ484540) were identified (Table II). Three putative cathelicidin peptide sequences were subsequently deduced and termed fowlicidins 1-3. Because the N-terminal sequence including the start codon of fowlicidin-2 was missing in GenBank, a genome walking approach known as vectorette PCR was performed by using chicken genomic DNA as previously described (30,31). As a result, a 1.8-kb upstream sequence of the fowlicidin-2 gene was obtained following two rounds of vectorette PCR (data not shown). The missing N-terminal peptide sequence of fowlicidin-2 was predicted by GenomeScan (29) based on its homology with the other two fowlicidins.

TABLE II
Identification of chicken fowlicidins

| | | | Gene Size (bp) | | | | | | |
|---|---|---|---|---|---|---|---|---|---|
| Gene | EST | WGS | E 1 | I 1 | E 2 | I 2 | E 3 | I 3 | E 4 |
| fowlicidin-1 | BX936022 | AADN01081708 | 168 | 537 | 108 | 84 | 84 | 99 | 87 |
| | BU106516 | | | | | | | | |
| | AY534900 | | | | | | | | |
| fowlicidin-2 | AJ393748 | AADN01005055 | 168 | 901 | 108 | 70 | 84 | 293 | 105 |
| | BU420865 | | | | | | | | |
| | CB018183 | | | | | | | | |
| fowlicidin-3 | CR389785 | AADN01005055 | 168 | 535 | 108 | 84 | 84 | 99 | 96 |
| | | AADN01005056 | | | | | | | |

The chicken genome contains three cathelicidin genes, namely fowlicidins 1-3, encoded by expressed sequence tags (EST) and whole genome shotgun sequences (WGS) as indicated. Each fowlicidin gene consists of four exons (E) separated by three introns (I). The sizes of the first and last exons are given according to the coding sequences without the 5′- and 3′-untranslated regions being counted. Note that the first three exons are 100% identical between fowlicidin-1 and fowlicidin-3, except for exon 4, which encodes different mature sequences.

Alignment of three fowlicidin peptide sequences revealed that they are highly homologous to each other (FIG. 1). Among all three peptides, fowlicidin-1 and -3 are more closely related with >90% identity throughout the entire sequence. Chicken cathelicidins also share a high degree of similarity with all known mammalian cathelicidins, particularly in the prosequence region (FIG. 1). Noticeably, four cysteines that are conserved in the cathelin domain of all mammalian cathelicidins are also invariantly spaced in three fowlicidins. These results clearly suggest that three chicken fowlicidins are bona fide non-mammalian cathelicidins.

Despite of sequence conservation at the N-terminus, fowlicidins and classic cathelicidins are drastically diverged at the C-terminus (FIG. 1). Similar to classic cathelicidins, fowlicidins 1-3 are positively charged at the C-terminus due to the presence of an excess number of cationic residues (R and K). The preferred cleavage site for elastase in the processing and maturation of bovine and porcine cathelicidins (34,35) also appears to be conserved in the chicken. Therefore, mature fowlicidins 1-3 are predicted to be devoid of cysteines and composed of 26, 32, and 29 amino acid residues with a net charge of +8, +10, and +7, respectively (FIG. 1).

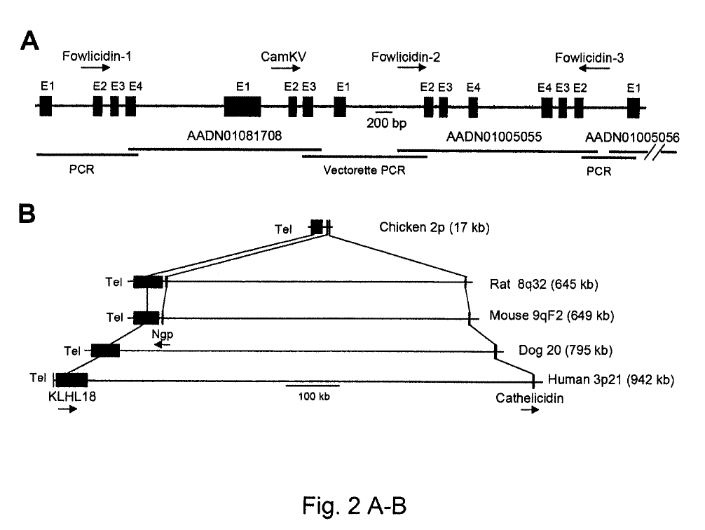

In addition to fowlicidins, we also identified the orthologs of rabbit P15 (22) and mouse NGP (23) in the rat, pig, and cow, which we named rNGP, pNGP, and bNGP, respectively (FIG. 1). However, no NGP-like genes were found in dogs or primates. Surprisingly, fowlicidins share a higher degree of similarity particularly in the signal sequence region with NGPs than to classic cathelicidins (FIG. 1), implying that chicken cathelicidins and NPGs may be more closely related. It is noted that all NGPs are highly conserved throughout the entire sequence among rodents and ungulates with a net negative charge at the C-terminus. The functional significance of such anionic sequences remains to be studied.
Genomic Organization of the Chicken Fowlicidin Gene Cluster—A screening through genomic sequences in the NR, HTGS and WGS databases in GenBank identified three WGS sequences. AADN01081708 contains a part of the last exon sequence of the fowlicidin-1 gene, and AADN01005055 and AADN01005056 encode the majority of the fowlicidin-2 and -3 genes (FIG. 2A). The fowlicidin-1 gene was cloned from chicken genomic DNA by PCR using the primers located in the first and last exons, whereas the missing first intron sequence of the fowlicidin-3 gene, i.e., the gap between AADN01005055 and AADN01005056, was cloned directly by PCR with primers located in two flanking exons (FIG. 2A). The 5′-end of the fowlicidin-2 gene was cloned by two rounds of vectorette PCR as described in the previous section. Structural organizations of three fowlicidin genes were obtained by comparing their cDNA with genomic DNA sequences. As shown in Table II, all three genes are organized similarly with four exons separated by three introns. The first three exons encode the signal and cathelin pro-sequences, whereas the last exon primarily encodes the mature sequences. Such structures are surprisingly identical to the mammalian cathelicidin genes, a clear indication of significant conservation during evolution.

Annotation of AADN01005055, AADN01005056, AADN01081708, and our newly cloned intra- and intergenic sequences resulted in the formation a continuous, gap-free contig that harbors all three fowlicidin genes, which has been deposited to GenBank under accession number DQ092350. As shown in FIG. 2A, three fowlicidin genes are packed tightly in a 7.5-kb distance on the chromosome with fowlicidin-2 and -3 in a head-to-head orientation that is separated only by 736 by from the stop codons of both genes. However, fowlicidin-1 and -2 are separated 2.4 kb from each other by a gene homologous to the C-terminal end of vesicle-associated, calmodulin kinase-like kinase (CamKV) (GenBank accession no. NP 076951). Chromosomal location of the chicken cathelicidin gene cluster was further revealed by using the Map Viewer Program. AADN01005055 and AADN01005056 were found to locate on the p arm of chromosome 2 that are less than 3.5 Mb from the proximal end in the current chicken genome assembly (Build 1.1) released on Jul. 1, 2004, but AADN01081708 remains unmapped.
Comparative and Evolutionary Analyses of Vertebrate Cathelicidins—Identification of three chicken fowlicidins provides an excellent opportunity to study the evolutionary relationship of mammalian cathelicidins. The physical locations of the cathelicidin gene clusters across several phylogenetically distant vertebrate species were examined. As shown in FIG. 2B, the cathelicidin genes are located in the syntenic regions flanking an evolutionarily conserved gene, Kelch-like 18 (KLHL18) (NP_{—}071737) across rodents, dogs, and humans, clearly indicating that cathelicidins in mammals and birds share a common ancestor. It is noteworthy that the chicken CamKV gene is absent in syntenic regions in mammals (FIG. 2B).

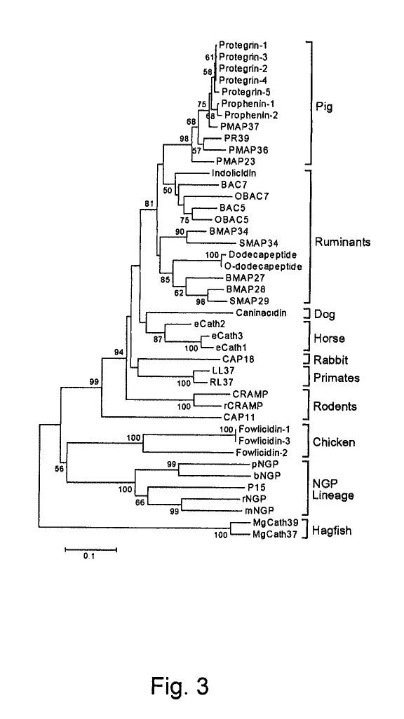

We then performed the phylogenetic analysis of fowlicidins together with all known mammalian cathelicidins and two recently identified hagfish cathelicidins using the neighbor-joining method (33) by calculating the proportion of amino acid differences. All vertebrate cathelicidins clearly formed three distinct clusters with two hagfish peptides located in a separate clade from others (FIG. 3). Supported by a bootstrap value of 56%, fowlicidins clustered with NGPs, suggesting that fowlicidins and NGPs are likely to originate from a common ancestor prior to the separation of birds from mammals. This is further supported by the close proximity of fowlicidins and NGPs with KLHL18 on chromosomes (FIG. 2B). All classic mammalian cathelicidins comprised a separate cluster supported by a bootstrap value of 99% (FIG. 3) and are located more than 500 kb away from KLHL18 and NGP, implying that classic cathelicidins are likely to be duplicated from NGPs after the mammal-bird split. Apparent absence of NGPs in the dog, chimpanzee, and human genomes (data not shown) suggested that the NGP lineage was lost after canines and primates diverged from other mammals.

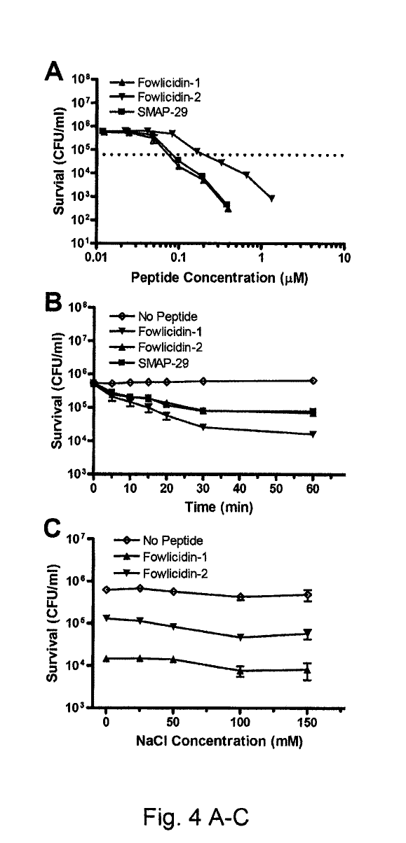

However, it is also possible that two different primordial genes for NGPs/fowlicidins and classic cathelicidins were present in the common ancestor of ayes and mammals. Both gene lineages are preserved in most mammals, but the classic cathelicidin lineage was lost in ayes after they split from mammals. Because two hagfish cathelicidins are too divergent, it is impossible to point out the evolutionary relationship of fish cathelicidins with their avian and mammalian homologs. Availability of genomic sequences of additional phylo-genetically distant lower vertebrate species is expected to help bridge the gap.
Antibacterial Properties of Fowlicidins—To test antibacterial properties of chicken cathelicidins, putatively mature fowlicidin-1 and -2 were synthesized commercially by the standard solid phase synthesis method and purified to >95% purity. A reference strain of E. coli ATCC 25922 was tested by using the colony counting assay as previously described (36). As shown in FIG. 4A, fowlicidin-1 and -2 displayed a MIC₉₀ of 65 nM and 180 nM, respectively, against E. coli. In fact, when compared directly with SMAP-29, which is the most potent cathelicidin that has been reported thus far (7), both fowlicidin-1 and -2 showed comparable antibacterial potency, implying the promising therapeutic potential of these two chicken cathelicidins. Furthermore, similar to SMPA-29, both fowlicidins showed a rapid killing of E. coli with the maximum killing occurring at 30 min at MIC₉₀ concentrations (FIG. 4B), reinforcing the notion that both fowlicidins kill bacteria most likely through physical membrane disruption. However, unlike many antimicrobial peptides whose antimicrobial activities are inhibited by salt at physiological concentrations (36-38,43,44), fowlicidin-1 and -2 maintained their activities up to 150 mM NaCl (FIG. 4C), implying their potential for systemic therapeutic applications. Interestingly, no obvious synergistic effect of two fowlicidin peptides in killing E. coli was observed when applied together (data not shown).

To test the antibacterial spectrum of fowlicidins, six Gram-negative and four Gram-positive bacterial strains were used in a modified broth microdilution assay (37). Both chicken cathelicidins were broadly effective against all bacteria tested in a salt-independent manner, with most MIC values in the range of 0.4-2.0 μM (Table III). P. aeruginosa appeared to be the only exception, being slightly more resistant to fowlicidins with the MIC of 3-6 μM for both peptides. Strikingly, both peptides displayed comparable antibacterial potency with SMAP-29 against all bacteria, although there was a tendency that fowlicidin-1 is slightly more efficacious than fowlicidin-2 in most cases (Table III). More desirably, both peptides were equally effective against antibiotic-resistant bacterial strains, such as multidrug-resistant S. typhimurium DT104 and methicilin-resistant S. aureus (MRSA) (Table III). Fowlicidin-3 also showed similar antibacterial activities against Gram-positive and Gram-negative bacteria to fowlicidin-1 (data not shown), but was omitted for further functional analyses because of its high homology with fowlicidin-1 (FIG. 1).

TABLE III
Antibacterial spectrum of fowlicidins against
Gram-negative and Gram-positive bacteria.

| | | MIC (μM) | | | | | |
|---|---|---|---|---|---|---|---|
| | | Fowlicidin-1 | | Fowlicidin-2 | | SMAP-29 | |
| | | (NaCl, mM) | | (NaCl, mM) | | (NaCl, mM) | |
| Bacteria | ATCC # | 0 | 100 | 0 | 100 | 0 | 100 |
| Gram-negative: | | | | | | | |
| E. coli | 25922 | 1.59 | 1.59 | 2.66 | 2.66 | 1.56 | 1.56 |
| E. coli O157:H7 | 700408 | 0.80 | 0.80 | 0.66 | 1.33 | 0.78 | 0.78 |
| S. typhimurium | 14028 | 0.80 | 1.59 | 1.33 | 1.33 | 0.39 | 0.78 |
| S. typhimurium | 700408 | 0.40 | 1.59 | 0.66 | 1.33 | 0.39 | 0.78 |
| DT104 | | | | | | | |
| K. pneumoniae | 13883 | 0.40 | 0.80 | 0.66 | 1.33 | 0.39 | 0.39 |
| P. aeruginosa | 27853 | 3.18 | 3.18 | 5.32 | 2.66 | 3.12 | 3.12 |
| Gram-positive: | | | | | | | |
| L. monocytogenes | 19115 | 0.80 | 1.59 | 1.33 | 1.33 | 0.78 | 0.78 |
| S. aureus | 25923 | 0.80 | 0.80 | 0.66 | 1.33 | 0.78 | 0.78 |
| S. aureus | BAA-39 | 0.40 | 0.80 | 0.66 | 1.33 | 0.39 | 0.39 |
| (MRSA)¹ | | | | | | | |
| S. aureus | 43300 | 0.40 | 0.40 | 0.33 | 2.66 | 0.39 | 0.39 |
| (MRSA)¹ | | | | | | | |

Mid-log phase bacteria (5 × 10⁵ CFU/ml) in 1% cation-adjusted Muller Hinton broth (MHB) were incubated overnight with serial twofold dilutions of peptides in the presence or absence of 100 mM NaCl. The exception was P. aeruginosa ATCC 27853, which was incubated with peptides in 10% cation-adjusted MHB with or without addition of 100 mM NaCl. The minimum inhibitory concentrations (MIC) of individual peptides against each bacterial strain were determined as the peptide concentration that gave no visible bacterial growth after overnight incubation. The experiments were repeated at least twice with essentially the same results.
¹MRSA, methicilin-resistant S. aureus.

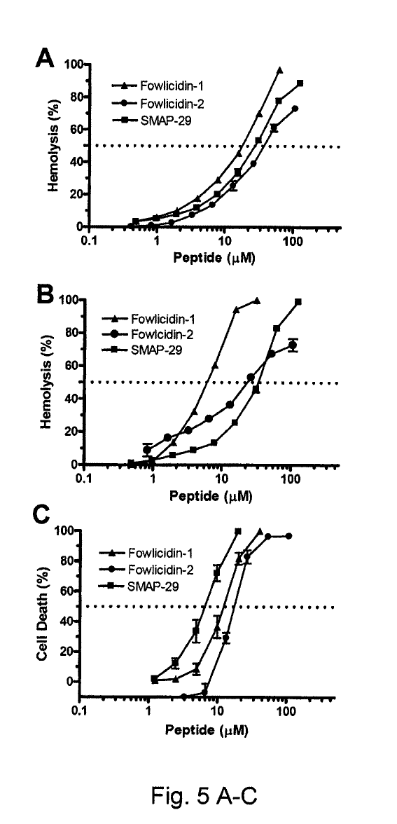

Cytotoxicity of Fowlicidins—To evaluate the hemolytic activity of fowlicidins against red blood cells, freshly isolated human and chicken erythrocytes were incubated with fowlicidins, together with SMAP29 as a positive reference. Hemolysis was monitored by measuring the absorbance at 405 nm for released hemoglobin as described (38, 39). Hemolytic activities of both chicken cathelicidins were similar toward human and chicken erythrocytes with EC₅₀ occurring at approximately 6-10 μM and 15-20 μM for fowlicidin-1 and -2, respectively (FIGS. 5A and 5B). Hemolytic activities of both fowlicidins and SMAP-29 were reduced by 2-4 fold in the presence of 10% FBS (data not shown).

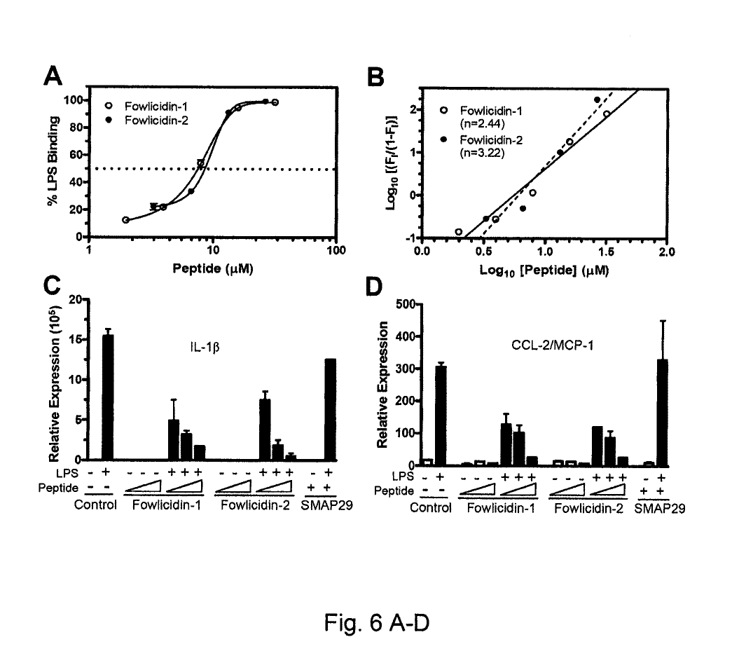

To further examine the cytotoxicities of fowlicidins toward mammalian epithelial cells, the viability of MDCK cells was measured by an alamarBlue-based, colorimetric method (40), following exposure to either peptide for 24 h. As shown in FIG. 5C, in the absence of 10% FBS, EC₅₀ occurred in the range of 10-20 μM toward MDCK cells for both fowlicidins, with SMAP-29 being the most toxic. A similar trend also occurred with RAW264.7 macrophage cells (data not shown). A 2- to 5-fold reduction in toxicity was observed in the presence of 10% FBS for all three peptides. It is noted that the cytotoxicities of fowlicidins toward mammalian host cells are at least several fold higher than their MIC values toward bacteria (compare Table III and FIG. 5), suggestive of the therapeutic potential of the fowlicidins. Nevertheless, a further reduction of their toxicity through rational mutagenesis will be desirable.
LPS Binding and Host Gene Modulatory Activities of Fowlicidins—To test the potential of fowlicidins as anti-sepsis agents, a chromogenic Limulus amebocyte lysate assay was used to measure the binding of LPS to fowlicidins by the competitive inhibition of LPS-induced procoagulant activation as described (37,41). As shown in FIG. 6A, typical sigmoidal curves of LPS binding activity were observed for both peptides, which exhibited a similar LPS binding efficiency with 50% binding occurring at 7.5 μM and 8.6 μM for fowlicidin-1 and -2, respectively. Both peptides completely inhibited the LPS procoagulant activity at 10-15 μM concentrations. Because the sigmoidal shapes imply cooperativity, we also graphed the data on a Hill Plot (FIG. 6B). The Hill plot coefficients (slopes) were calculated to be 2.44 and 3.22 for fowlicidin-1 and -2, respectively, suggesting the presence of cooperative LPS binding sites possibly on each peptide molecule for both peptides. These results are reminiscent of SMAP-29 and LL-37 with multiple intramolecular LPS binding sites that function cooperatively to allow peptides to bind to LPS with high affinity (37,41).

To further evaluate whether binding of fowlicidins to LPS can neutralize LPS-induced proinflammatory responses, RAW264.7 macrophage cells were stimulated with LPS in the presence of different concentrations of peptides, followed by evaluation of proinflammatory cytokine/chemokine gene expression by real time reverse transcriptase-PCR. Fowlicidin-1 and -2, when applied up to 20 μM in the absence of LPS, did not alter gene expression. However, they blocked LPS-induced expression of IL-1β and CCL-2/MCP-1 in a dose-dependent fashion (FIGS. 6C and 6D). The same trend also occurred with CCL-3/MIP-1a for both peptides (data not shown). In fact, fowlicidins inhibited the expression of all three genes by >90% at 20 μtM (FIGS. 6C and 6D) Surprisingly, albeit with in vitro LPS-binding activity (41), SMAP-29, even at 20 μM, failed to suppress the expression of any of the three proinflammatory genes that were examined. Collectively, these data strongly suggested the potential of fowlicidin-1 and -2 as both antibacterial and anti-sepsis agents. It is noted that all three peptides, when applied at 20 μM, caused only minimal, <5% cell death to RAW264.7 cells in the presence of 10% FBS (data not shown).

### Discussion

Three chicken cathelicidins consist of linear cationic sequences at the C-termini, which are expected to be freed from the cathelin domain to become biologically active. Indeed, putatively mature fowlicidins possess potent antibacterial activities (FIG. 4 and Table III). Although valine on the fourth exon of fowlicidins (FIG. 1) is likely to be the processing site for chicken elastase-like protease as in the case of bovine and porcine cathelicidins (34,35), the protease and exact cleavage site for fowlicidins need to be experimentally confirmed.

In the course of screening for chicken cathelicidins, NGPs were also identified in rats, pigs and cows that are highly homologous to P15s in rabbits (22) and NGP in mice (23) (FIG. 1). These NGP-like proteins appear to be evolutionarily conserved only in glires and ungulates, but not in dogs and primates. In spite of relatively low homology in the cathelin domain with the majority of other mammalian cathelicidins, NGPs share similar tissue expression pattern (22,23), chromosomal location (FIG. 2B), gene structure (data not shown), and antimicrobial activities (22) to classic mammalian cathelicidins, and therefore, clearly belong to the cathelicidin family. Identification of three fowlicidins, which are more closely related to NGPs (FIGS. 1 and 3), suggested that the ancestral gene for fowlicidins/NGPs arose in the common ancestor of mammals and birds, which may have further given rise to classic mammalian cathelicidins as a result of gene duplication after the mammal-bird split. Classic cathelicidins must have been duplicated from NGPs prior to the divergence of mammals from each other, because of a high degree of homology within classic cathelicidins particularly in the cathelin domain. Apparently, independent duplications have occurred after mammals were separated from each other, which is supported by species-specific clustering and presence of a varied number of classic cathelicidins in most cases (FIG. 3). For example, ungulates have multiple cathelicidins, whereas most other mammals have one or very few members.

As for the origin of three chicken cathelicidins, fowlicidin-1 and -3 are apparently a result of gene duplication, because of significant homology across the entire open reading frame (FIG. 1). Furthermore, the intron sequences of these two cathelicidin genes are highly similar (data not shown). Fowlicidin-2 also shares significant homology in the first three exons with fowlicidin-1 and -3, but diverged greatly in the last exon encoding the mature sequence (FIG. 1). However, alignment of the last intron and exon nucleotide sequences of three fowlicidin genes revealed approximately 45% identity (data not shown). This suggested that, the entire fowlicidin-2 gene may have been duplicated directly from fowlicidin-1 or -3, but not a result of exon shuffling (45), which probably gave rise to multiple cathelicidins with drastic sequence divergence in the last exon in ungulates. Although P15s is unique in that its does not undergo proteolytic processing when released (22), it will be interesting to see whether it is also true with other mammalian NGPs. The presence of valines in the fourth exons of NGPs in the pig and rat at equivalent elastase cleavage site raises the possibility that at least some NGPs may be enzymatically processed upon activation (FIG. 1). However, the C-terminal peptides of NGPs are all negatively charged, as opposed to classic cathelicidins with cationic sequences. Therefore, it will be interesting to study the processing and biological roles of these NGPs. Because of the existence of two cathelicidins (NGP and CRAMP) in mice as opposed to a single cathelicidin (LL-37/hCAP-18) in humans, extrapolation of the data from CRAMP-deficient mouse to the human system needs to be more prudent.

During the preparation of this manuscript, sequences identical to fowlicidin-1 and -2 have been reported independently (46,47). Similar to most mammalian cathelicidins, fowlicidin-1 and -2 were shown to be expressed primarily in bone marrow, but none of the functional information was been provided (46,47). Our data clearly indicated that putatively mature fowlicidins are clearly among the most potent cathelicidins discovered to date, killing a variety of bacteria at <2 μM (Table III). This is perhaps due to the fact that chicken heterophils, which are equivalent to mammalian neutrophils and likely the store sites of fowlicidins, lack myeloperoxidase and depends primarily on non-oxidative mechanisms for antimicrobial activity (48). Unlike many cationic antimicrobial peptides that are inactivated at physiological concentrations of salt, fowlicidins maintained antibacterial activity in the presence of high NaCl (FIG. 4C and Table III). Salt-independent killing of bacteria of fowlicidins may offer an attractive therapeutic option for cystic fibrosis and Crohn's disease, both of which are associated with aberrant local expression or inactivation of antimicrobial peptides (49-51).

In summary, discovery and functional characterization of these chicken cathelicidins offer new insights on the evolution of mammalian cathelicidins. Potent, broad-spectrum, salt-independent antibacterial activities with strong LPS-neutralizing activity make fowlicidins excellent candidates as antimicrobial and anti-sepsis agents.

### References for Example 1

1. Yang, D., Biragyn, A., Hoover, D. M., Lubkowski, J., and Oppenheim, J. J. (2004) Annu Rev Immunol 22, 181-215
2. Brogden, K. A., Ackermann, M., McCray, P. B., Jr., and Tack, B. F. (2003) Int J Antimicrob Agents 22(5), 465-478
3. Zasloff, M. (2002) Nature 415(6870), 389-395
4. Scott, M. G., and Hancock, R. E. (2000) Crit Rev Immunol 20(5), 407-431
5. Ganz, T. (2003) Nat Rev Immunol 3(9), 710-720
6. Lehrer, R. I. (2004) Nat Rev Microbiol 2(9), 727-738
7. Zanetti, M., Gennaro, R., Skerlavaj, B., Tomasinsig, L., and Circo, R. (2002) Curr Pharm Des 8(9), 779-793
8. Zaiou, M., and Gallo, R. L. (2002) J Mol Med 80(9), 549-561
9. Zanetti, M. (2004) J Leukoc Biol 75(1), 39-48
10. Zarember, K. A., Katz, S. S., Tack, B. F., Doukhan, L., Weiss, J., and Elsbach, P. (2002) Infect Immun 70(2), 569-576
11. Zaiou, M., Nizet, V., and Gallo, R. L. (2003) J Invest Dermatol 120(5), 810-816
12. Ong, P. Y., Ohtake, T., Brandt, C., Strickland, I., Boguniewicz, M., Ganz, T., Gallo, R. L., and Leung, D. Y. (2002) N Engl J Med 347(15), 1151-1160
13. Putsep, K., Carlsson, G., Boman, H. G., and Andersson, M. (2002) Lancet 360(9340), 1144-1149
14. Nizet, V., Ohtake, T., Lauth, X., Trowbridge, J., Rudisill, J., Dorschner, R. A., Pestonjamasp, V., Piraino, J., Huttner, K., and Gallo, R. L. (2001) Nature 414(6862), 454-457
15. Iimura, M., Gallo, R. L., Hase, K., Miyamoto, Y., Eckmann, L., and Kagnoff, M. F. (2005) J Immunol 174(8), 4901-4907
16. Carretero, M., Del Rio, M., Garcia, M., Escamez, M. J., Mirones, I., Rivas, L., Balague, C., Jorcano, J. L., and Larcher, F. (2004) FASEB J 18(15), 1931-1933
17. Bartlett, K. H., McCray, P. B., Jr., and Thorne, P. S. (2003) Antimicrob Agents Chemother 47(12), 3901-3906
18. Lee, P. H., Ohtake, T., Zaiou, M., Murakami, M., Rudisill, J. A., Lin, K. H., and Gallo, R. L. (2005) Proc Natl Acad Sci USA 102(10), 3750-3755
19. Bals, R., Weiner, D. J., Meegalla, R. L., and Wilson, J. M. (1999) J Clin Invest 103(8), 1113-1117
20. Bals, R., Weiner, D. J., Moscioni, A. D., Meegalla, R. L., and Wilson, J. M. (1999) Infect Immun 67(11), 6084-6089
21. Shai, Y. (2002) Curr Pharm Des 8(9), 715-725
22. Levy, O., Weiss, J., Zarember, K., Ooi, C. E., and Elsbach, P. (1993) J Biol Chem 268(8), 6058-6063
23. Moscinski, L. C., and Hill, B. (1995) J Cell Biochem 59(4), 431-442
24. Uzzell, T., Stolzenberg, E. D., Shinnar, A. E., and Zasloff, M. (2003) Peptides 24(11), 1655-1667
25. Altschul, S. F., Gish, W., Miller, W., Myers, E. W., and Lipman, D. J. (1990) J Mol Biol 215(3), 403-410
26. Xiao, Y., Hughes, A. L., Ando, J., Matsuda, Y., Cheng, J. F., Skinner-Noble, D., and Zhang, G. (2004) BMC Genomics 5(1), 56
27. Patil, A., Hughes, A. L., and Zhang, G. (2004) Physiol Genomics 20(1), 1-11
28. Patil, A. A., Cai, Y., Sang, Y., Blecha, F., and Zhang, G. (2005) Physiol Genomics 23(1), 5-17
29. Yeh, R. F., Lim, L. P., and Burge, C. B. (2001) Genome Res 11(5), 803-816
30. Munroe, D. J., Haas, M., Bric, E., Whitton, T., Aburatani, H., Hunter, K., Ward, D., and Housman, D. E. (1994) Genomics 19(3), 506-514
31. Riley, J., Butler, R., Ogilvie, D., Finniear, R., Jenner, D., Powell, S., Anand, R., Smith, J. C., and Markham, A. F. (1990) Nucleic Acids Res 18(10), 2887-2890
32. Thompson, J. D., Higgins, D. G., and Gibson, T. J. (1994) Nucleic Acids Res 22(22), 4673-4680
33. Saitou, N., and Nei, M. (1987) Mol Biol Evol 4(4), 406-425
34. Scocchi, M., Skerlavaj, B., Romeo, D., and Gennaro, R. (1992) Eur J Biochem 209(2), 589-595
35. Panyutich, A., Shi, J., Boutz, P. L., Zhao, C., and Ganz, T. (1997) Infect Immun 65(3), 978-985
36. Shi, J., Zhang, G., Wu, H., Ross, C., Blecha, F., and Ganz, T. (1999) Infect Immun 67(6), 3121-3127
37. Turner, J., Cho, Y., Dinh, N. N., Waring, A. J., and Lehrer, R. I. (1998) Antimicrob Agents Chemother 42(9), 2206-2214
38. Shin, S. Y., Park, E. J., Yang, S. T., Jung, H. J., Eom, S. H., Song, W. K., Kim, Y., Hahm, K. S., and Kim, J. I. (2001) Biochem Biophys Res Commun 285(4), 1046-1051
39. Yu, K., Park, K., Kang, S. W., Shin, S. Y., Hahm, K. S., and Kim, Y. (2002) J Pept Res 60(1), 1-9
40. Nociari, M. M., Shalev, A., Benias, P., and Russo, C. (1998) J Immunol Methods 213(2), 157-167
41. Tack, B. F., Sawai, M. V., Kearney, W. R., Robertson, A. D., Sherman, M. A., Wang, W., Hong, T., Boo, L. M., Wu, H., Waring, A. J., and Lehrer, R. I. (2002) Eur J Biochem 269(4), 1181-1189
42. Hettinger, A. M., Allen, M. R., Zhang, B. R., Goad, D. W., Malayer, J. R., and Geisert, R. D. (2001) Biol Reprod 65(2), 507-513
43. Travis, S. M., Anderson, N. N., Forsyth, W. R., Espiritu, C., Conway, B. D., Greenberg, E. P., McCray, P. B., Jr., Lehrer, R. I., Welsh, M. J., and Tack, B. F. (2000) Infect Immun 68(5), 2748-2755
44. Larrick, J. W., Hirata, M., Balint, R. F., Lee, J., Zhong, J., and Wright, S. C. (1995) Infect Immun 63(4), 1291-1297
45. Patthy, L. (1999) Gene 238(1), 103-114
46. Lynn, D. J., Higgs, R., Gaines, S., Tierney, J., James, T., Lloyd, A. T., Fares, M. A., Mulcahy, G., and O'Farrelly, C. (2004) Immunogenetics 56(3), 170-177
47. van Dijk, A., Veldhuizen, E. J., van Asten, A. J., and Haagsman, H. P. (2005) Vet Immunol Immunopathol 106(3-4), 321-327
48. Harmon, B. G. (1998) Poult Sci 77(7), 972-977
49. Kobayashi, K. S., Chamaillard, M., Ogura, Y., Henegariu, O., Inohara, N., Nunez, G., and Flavell, R. A. (2005) Science 307(5710), 731-734
50. Wehkamp, J., Fellermann, K., and Stange, E. F. (2005) Chem Immunol Allergy 86, 42-54
51. Smith, J. J., Travis, S. M., Greenberg, E. P., and Welsh, M. J. (1996) Cell 85(2), 229-236

### Example 2

Cathelicidins are a major family of animal antimicrobial peptides with hallmarks of a highly conserved prosequence (cathelin domain) and an extremely variable, antibacterially active sequence at the C-terminus [1-3]. The exact microbicidal mechanism for this family of antimicrobial peptides is not clearly understood. However, it is generally believed that the electrostatic interaction between the C-terminal cationic peptides with anionic lipids followed by membrane permeabilization is mainly responsible for killing prokaryotic cells. Because of such a non-specific membrane-lytic mechanism, many cathelicidins kill a variety of bacteria at low micromolar concentrations with much less chance of developing resistance [4-6]. More importantly, they are equally active against antibiotic-resistant bacterial strains with some demonstrating synergism in killing bacteria with conventional antibiotics or structurally different antimicrobial peptides [7-9]. One side-effect that is commonly associated with cathelicidins as potential therapeutic agents is their cytotoxicity toward mammalian host cells [4-6]. However, the concentrations that are required for cathelicidins to exert an appreciable cytolytic effect are often higher than the bactericidal concentrations. Structure-activity relationship (SAR) studies of cathelicidins revealed that cationicity, amphipathicity, hydrophobicity, and helicity (helical content) are among the most important determinants of their microbicidal and cytolytic activities [10, 11]. However, in general there is no simple correlation between any of these physicochemical properties and peptide functions. A delicate balance of these parameters often dictates the antimicrobial potency and target selectivity [10, 11]. Moreover, the domain that is responsible for cytotoxicity can sometimes be separated from that for antimicrobial activity [12, 13]. Therefore, it is possible that strategic manipulation of structural and physicochemical parameters of cathelicidins may maximize their antimicrobial activity while reducing their cytotoxicity.

Three novel chicken cathelicidins have been identified [14-16], which are called fowlicidins 1-3 herein. All three fowlicidins share little similarity with mammalian cathelicidins in the C-terminal sequence [16]. Putatively mature fowlicidin-1, a linear peptide of 26 amino acid residues, is broadly active against a range of Gram-negative and Gram-positive bacteria with a similar potency to SMAP-29 [16]. However, fowlicidin-1 also displayed considerable cytotoxicity toward human erythrocytes and mammalian epithelial cells with 50% lysis in the range of 6-40 μM [16].

To better understand its mechanism of action, we determined its tertiary structure by NMR spectroscopy in this study. Fowlicidin-1 was shown to be composed of an α-helical segment with a slight kink near the center and a flexible unstructured region at the N-terminal end. A series of deletion and substitution mutants of fowlicidin-1 were further synthesized and tested separately for their antibacterial, LPS-binding, and cytolytic activities. The regions that are responsible for each of these functions have been revealed. In addition, we identified a fowlicidin-1 analog with deletion of the N-terminal flexible region that retains the antibacterial potency but with substantially reduced cytotoxicity. Such a peptide analog may represent an excellent candidate as a novel antimicrobial agent against bacteria that are resistant to conventional antibiotics.

### Materials and Methods

### Peptide Synthesis

Fowlicidin-1 was synthesized using the standard solid-phase method by SynPep (Dublin, Calif.) and its analogs were synthesized by either Sigma Genosys (Woodlands, Tex.) or Bio-Synthesis (Lewisville, Tex.). The peptides were purified through RP-HPLC and purchased at >95% purity. The mass and purity of each peptide were further confirmed by 15% Tris-Tricine polyacrylamide gel electrophoresis (data not shown) and by MALDI-TOF mass spectrometry (not shown) using the Voyager DE-PRO instrument (Applied Biosystems, Foster City, Calif.) housed in the Recombinant DNA/Protein Resource Facility of Oklahoma State University.

### CD Spectroscopy

To determine the secondary structure of fowlicidin-1, CD spectroscopy was performed with a Jasco-715 spectropolarimeter using a 0.1-cm path length cell over the 180-260 nm range as we previously described [41]. The spectra were acquired at 25° C. every 1 nm with a 2-s averaging time per point and a 1-nm band pass. Fowlicidin-1 (10 μM) was measured in 50 mM potassium phosphate buffer, pH 7.4, with or without different concentrations of TFE (0%, 10%, 20%, 40%, and 60%) or SDS micelles (0.25% and 0.5%). Mean residue ellipticity (MRE) was expressed as [θ]MRE (deg.cm².dmol⁻¹). The contents of six types of the secondary structural elements, including regular and distorted α-helix, regular and distorted β-sheet, turns, and unordered structures, were analyzed with the program SELCON3 [42].

### NMR Spectroscopy

2D [¹H-¹H] NMR experiments for fowlicidin-1 were performed as previously described [43, 44]. Briefly, NMR data were acquired on a 11.75T Varian UNITYplus spectrometer (Varian, Palo Alto, Calif.), operating at 500 MHz for ¹H, with a 3-mm triple-resonance inverse detection probe. The NMR sample of fowlicidin-1, consisting of 4 mM in water containing 50% deuterated TFE (TFE-d3, Cambridge Isotope Laboratories) and 10% D₂O, was used to record spectra at 10, 20, 30, and 35° C. The spectra acquired at 35° C. were determined to provide the optimal resolution of overlapping NMR resonances. These spectra were processed and analyzed using Varian software VNMR Version 6.1C on a Silicon Graphics Octane workstation. The invariant nature of the NMR chemical shifts and line widths upon 10-fold dilution indicated that fowlicidin-1 was monomeric in solution at the concentration used for 2D NMR analysis. A total of 512 increments of 4K data points were collected for these 2D NMR experiments. The high digital resolution DQF-COSY spectra were recorded using 512 increments and 8K data points in t1 and t2 dimensions. Sequential assignments were carried out by comparison of cross-peaks in a NOESY spectrum with those in a TOCSY spectrum acquired under similar experimental conditions. NOESY experiments were performed with 200, 300, 400 and 500 ms mixing times. A mixing time of 200 ms was used for distance constraints measurements. The NOE cross-peaks were classified as strong, medium, weak and very weak based on an observed relative number of contour lines. TOCSY spectra were recorded by using MLEV-17 for isotropic mixing for 35 and 100 ms at a B1 field strength of 7 KHz.

Water peak suppression was obtained by low-power irradiation of the water peak during relaxation delay. The residual TFE methylene peak was considered as reference for the chemical shift values. The temperature dependencies of amide proton chemical shifts were measured by collecting data from 10° to 35° C. in steps of 5° C. by using a variable temperature probe. All experiments were zero-filled to 4K data points in t1 dimension and when necessary, spectral resolution was enhanced by Lorenzian-Gaussian apodization.

### Structure Calculations

For structure calculations, NOE-derived distance restraints were classified into four ranges: 1.8-2.7, 1.8-3.5, 1.8-4.0 and 1.8-5.0 Å, according to the strong, medium, weak and very weak NOE intensities. Upper distance limits for NOEs involving methyl protons and non-stereospecifically assigned methylene protons were corrected appropriately for center averaging [45]. In addition, a distance of 0.5 Å was added to the upper distance limits only for NOEs involving the methyl proton after correction for center averaging [46]. The distance restraints were then used to create initial peptide structures starting from extended structures using the program CNS (version 1.1) [47]. CNS uses both a simulated annealing protocol and molecular dynamics to produce low energy structures with the minimum distance and geometry violations. In general, default parameters supplied with the program were used with 100 structures for each CNS run. The final round of calculations began with 100 initial structures and 20 best structures with the lowest energy were selected and analyzed with MOLMOL [48] and PROCHECK-NMR [19]. Structure figures were generated by using MOLMOL. The structures of fowlicidin-1 analogs were further modeled by using Modeller [20], based on the parent peptide.

### Antibacterial Assay

Two representative Gram-negative bacteria (E. coli ATCC 25922 and S. enterica serovar Typhimurium ATCC 14028) and two Gram-positive bacteria (L. monocytogenes ATCC 19115 and S. aureus ATCC 25923) were purchased from ATCC (Manassas, Va.) and tested separately against fowlicidin-1 and its analogs by using a modified broth microdilution assay as described [16, 21]. Briefly, overnight cultures of bacteria were subcultured for additional 3-5 h at 37° C. in trypticase soy broth to the mid-log phase, washed with 10 mM sodium phosphate buffer, pH 7.4, and suspended to 5×10⁵ CFU/ml in 1% cation-adjusted Mueller Hinton broth (BBL, Cockeysville, Md.), which was prepared by a 1:100 dilution of conventional strength Mueller Hinton broth in 10 mM phosphate buffer. If necessary, 100 mM of NaCl were added to test the influence of salinity on antibacterial activity. Bacteria (90 μl) were then dispensed into 96-well plates, followed by addition in duplicate of 10 μl of serially diluted peptides in 0.01% acetic acid. After overnight incubation at 37° C., the MIC value of each peptide was determined as the lowest concentration that gave no visible bacterial growth. The antibacterial assays were repeated at least 3-4 times for each bacterial strain with <2-fold difference in MIC values in all cases, and therefore, representative MIC values were tabulated in Table V.

### Hemolysis Assay

The hemolytic activity of fowlicidin-1 and its mutants were determined essentially as described [13, 22]. Briefly, fresh anti-coagulated human blood was collected, washed twice with PBS, diluted to 0.5% in PBS, and 90 μl were dispensed into 96-well plates. Serial 2-fold dilutions of peptides were added in duplicate to erythrocytes and incubated at 37° C. for 2 h. Following centrifugation at 800×g for 10 min, the supernatants were transferred to new 96-well plates and monitored by measuring the absorbance at 405 nm for released hemoglobin. Controls for 0 and 100% hemolysis consisted of cells suspended in PBS only and in 1% Triton X-100, respectively. Percent hemolysis (%) was calculated as [(A_{405 nm, peptide}−A_{405 nm, PBS})/(A_{405 nm, 1% Triton X-100}−A_{405 nm, PBS})]×100. EC₅₀ of the hemolytic activity was defined as the peptide concentration that caused 50% lysis of erythrocytes.

### Cytotoxicity Assay

The toxic effect of fowlicidin-1 and its analogs on mammalian epithelial cells was evaluated with MDCK cells by using alamarBlue dye (Biosource) as previously described [16]. Briefly, cells were seeded into 96-well plates at 1.5×10⁵/well and allowed to grow overnight in DMEM containing 10% FBS. Cells were then washed once with DMEM, followed by addition of 90 μl of fresh DMEM, together with 10 μl of serially diluted peptides in 0.01% acetic acid in triplicate. After incubation for 18 h, 10 μl of alamarBlue dye were added to cells for 6 h at 37° C. in a humidified 5% CO₂ incubator before the fluorescence was read with excitation at 545 nm and emission at 590 nm. Percent cell death was calculated as [1−(F_{peptide}−F_{background})/(F_{acetic acid}−F_{background})]×100, where F_{peptide} is the fluorescence of cells exposed to different concentrations of peptides, F_{acetic acid} is the fluorescence of cells exposed to 0.01% acetic acid only, and F_{background} is the background fluorescence of 10% alamarBlue dye in cell culture medium without cells. Cytotoxicity (EC₅₀) of individual peptides was defined as the peptide concentration that caused 50% cell death.

### LPS Binding Assay

The binding of LPS to fowlicidin-1 and its analogs was measured by a kinetic chromogenic Limulus amebocyte lysate assay (Kinetic-QCL 1000 kit; BioWhittaker, Walkersville, Md.) as previously described [21, 25]. Briefly, 25 μl of serially diluted peptide were added in duplicate into 25 μl of E. coli O111:B4 LPS containing 0.5 endotoxin units/ml and incubated for 30 min at 37° C., followed by incubation with 50 μl of the amoebocyte lysate reagent for 10 min. The absorbance at 405 nm was measured at 10 and 16 min after addition of 100 μl of chromogenic substrate, Ac-Ile-Glu-Ala-Arg-p-nitroanilide. Percent LPS binding was calculated as [(ΔD1−ΔD2+ΔD3)/ΔD1]×100, where ΔD1 represents the difference in the optical density between 10 and 16 min for the sample containing LPS only, ΔD2 represents the difference in the optical density between 10 and 16 min for the samples containing LPS and different concentrations of peptides, and ΔD3 represents the difference in the optical density between 10 and 16 min for the samples containing different concentrations of peptides with no LPS. EC₅₀ of the LPS-binding activity was defined as the peptide concentration that inhibited LPS-mediated procoagulant activation by 50%.

### Protein Data Bank Accession Code

The atomic coordinates and structure factors of putatively mature fowlicidin-1 have been deposited under accession code 2AMN in the Protein Data Bank, Research Collaboratory for Structural Bioinformatics, Rutgers University, New Brunswick, N.J. (see the web site at www.rcsb.org).

### Results

### Solution Structure of Fowlicidin-1

To first determine the secondary structure of fowlicidin-1, CD spectroscopy was performed in the increasing concentrations of structure-promoting agents, trifluoroethanol (TFE) and SDS. Fowlicidin-1 was largely unstructured in the aqueous solution, but underwent a dramatic transition to a typical α-helical conformation with addition of TFE. The α-helical content of fowlicidin-1 increased dose-dependently from 10% in 50 mM phosphate buffer to 81% in 60% TFE, with a concomitant reduction of the random coiled structure. Significant α-helical content (81%) was similarly observed in the presence of 0.25% or 0.5% SDS (not shown).

Because of adoption of a well-defined structure in the presence of TFE or SDS, subsequent NMR experiments were done in 50% deuterated TFE. The spectra acquired at 35° C. gave good chemical shift dispersion with limited spectral overlap, enabling the assignment of most spin systems for fowlicidin-1 (not shown). The complete proton resonance assignments were obtained for the peptide using spin system identification and sequential assignments [17] from 2D NMR spectra recorded at 35° C. Some ambiguities due to overlapping signals were also solved by the comparative use of spectra recorded at 10° C. and 35° C. In these assignments, Hα(i)-Hδ(i+1:Pro) (d_{αδ}) or Hα(i)-Hα(i+1:Pro) (d_{αα}.) NOEs instead of daN were used for Pro⁷, which showed strong dad NOEs, indicating that Pro⁷ in fowlicidin-1 has trans configuration.

Stereo-specific assignments of β-methylene protons were obtained by using information on ³J_{HαHβ} coupling constants estimated qualitatively from short-mixing time TOCSY spectra combined with intra-residue NH—H_{β} and H_{α}—H_{β} NOEs. Qualitative analysis of short- and medium-range NOEs, ³J_{HNHα} coupling constants, and slowly-exchanging amide proton patterns was used to characterize the secondary structure of fowlicidin-1. A number of nonsequential d_{αN}(i, i+3) and d_{αβ}(i, i+3) NOEs that are clearly characteristics of α-helical conformation were observed for fowlicidin-1 from Leu⁸ to Lys²⁵. A continuous stretch of d_{NN}(i, i+1) also extended from Leu⁸ to Lys²⁵, except for Gly¹⁶. The helicity of fowlicidin-1 was further supported by the chemical shift index (not shown).

To determine the tertiary structure of fowlicidin-1, a total of 247 NOE distance constraints involving 90 interresidue, 81 sequential, and 76 medium range constraints were used in structural calculations (not shown). Of 100 conformers calculated, 20 structures with the lowest energy were retained for further analysis. All 20 structures were in good agreement with the experimental data, with no distance violations >0.3 Å and no angle violations >5°. A Ramachandran plot was also produced by PROCHECK-NMR [19] showing that 76.1% of the residues are in the most favored region and 21.8 and 1.1% in additional and generously allowed regions, respectively.

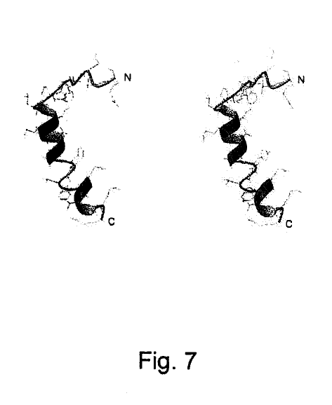

The minimized average structure (FIG. 7) indicated that fowlicidin-1 is primarily an α-helical peptide consisting of a helical segment from Leu⁸ to Lys²⁵ and a disordered region near the N-terminus from Arg¹ to Pro⁷. No unambiguous long range NOEs for the first four N-terminal residues were observed, indicative of their extremely flexible nature. A closer examination revealed that the long helix of fowlicidin-1 is further composed of two short, but perfect, α-helical segments (Leu⁸-Ala¹⁵ and Arg²¹-Lys²⁵) with a slight bend between Gly¹⁶ and Tyr²⁰, due to the presence of Gly¹⁶ (FIG. 7). A superimposition of the backbones of 20 lowest energy structures best fitted to residues 8-16 or residues 17-25 indicated that the two short helices are highly rigid, but with some degree of flexibility in between not shown). The superimposition of the two short helical segments of the 20 final structures against an averaged structure resulted in a RMSD value of backbone <0.5 Å. Greater flexibility between the helices was revealed when only one segment of the helix was superimposed. It is noteworthy that the angle between the two helical axes could not be measured because of a lack of NOEs in the Gly¹⁶ region and fluidity between the two segments. However, flexibility of the “hinge” is somewhat restricted by the side chains of nearby residues, such as Tyr¹⁷ (FIG. 7).

### Design and Physicochemical Properties of Fowlicidin-1 Analogs

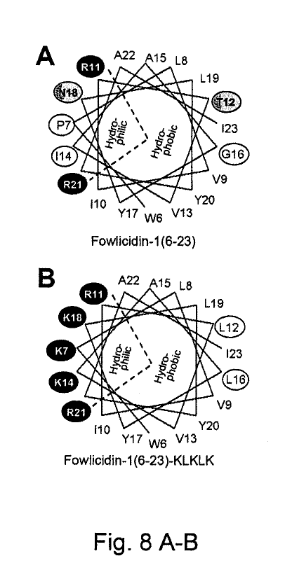

In contrast with most cathelicidins containing a highly cationic, amphipathic α-helix [10], the central helical region (residues 6-23) of fowlicidin-1 is highly hydrophobic, containing only two cationic residues (Arg¹¹ and Arg²¹) and two uncharged polar residues (Thr¹² and Gln¹⁸) (FIG. 8A). Positively charged residues are instead highly concentrated at both ends. To probe the impact of N- and C-terminal cationic regions and two short helical segments on antibacterial, LPS-binding, and cytolytic activities of fowlicidin-1, several N- and C-terminal deletion mutants were designed (Table IV). All mutants have fewer net positive charges than the parent peptide, in addition to missing one or two structural components.

TABLE IV
Fowlicidin-1 and its analogs

| | | | | Mass | |
|---|---|---|---|---|---|
| Peptide | Sequence | Charge | Length | Calculated | Observed |

| | | | | | |
|---|---|---|---|---|---|
| Fowlicidin-1 | RVKRVWPLVIRTVIAG | +8 | 26 | 3141.9 | 3141.6 |
| (1-26) | YNLYRAIKKK | | | | |
| | (SEQ ID NO: 2) | | | | |
| Fowl-1(1-15) | RVKRVWPLVIRTVIA | +4 | 15 | 1807.3 | 1807.6 |
| | (SEQ ID NO: 3) | | | | |
| Fowl-1(1-23) | RVKRVWPLVIRTVIAG | +5 | 23 | 2758.4 | 2757.2 |
| | YNLYRAI | | | | |
| | (SEQ ID NO: 4) | | | | |
| Fowl-1(8-26) | LVIRTVIAGYNLYRAI | +5 | 19 | 2220.8 | 2220.9 |
| | KKK | | | | |
| | (SEQ ID NO: 5) | | | | |
| Fowl-1(5-26) | VWPLVIRTVIAGYNLY | +5 | 22 | 2603.2 | 2600.3 |
| | RAIKKK | | | | |
| | (SEQ ID NO: 6) | | | | |
| Fowl-1-L16 | RVKRVWPLVIRTVIAL | +8 | 26 | 3199.0 | 3197.3 |
| | YNLYRAIKKK | | | | |
| | (SEQ ID NO: 7) | | | | |
| Fowl1- | RVKRVWKLVIRLVKA | +11 | 26 | 3271.2 | 3271.1 |
| K⁷L¹²K¹⁴L¹⁶K¹⁸ | LYKLYRAIKKK | | | | |
| | (SEQ ID NO: 8) | | | | |

To further investigate the influence of helicity on the functional properties, Gly¹⁶ of fowlicidin-1 was replaced with a helix-stabilizing residue, leucine, to give rise to fowlicidin-1-L¹⁶. Such a variant minimized the bend and flexibility between two short helices as modeled by Modeller [20] (data not shown), without significantly altering any other structural or physicochemical characteristics. Another substitution variant, , fowlicidin-1-K⁷L¹²K¹⁴L¹⁶K¹⁸, was designed mainly for significant augmentation of its amphipathicity. This mutant now has cationic residues clearly aligned along one side and hydrophobic residues aligned along the opposite side of the helix (compare FIGS. 8A with 8B). The net charge of this mutant has increased from +8 to +11, as compared with the parent peptide. Replacement of two helix-breaking residues, Pro⁷ and Gly¹⁶, with helix-stabilizing residues, lysine and leucine, respectively, also enhanced the helical content of fowlicidin-1-K⁷L¹²K¹⁴L¹⁶K¹ by concomitant reduction of the kink in the center and extension of the helix at the N-terminus. Along with simultaneous enhancement of amphipathicity, cationicity, and helicity, it is understandable that such a peptide variant also has reduced hydrophobicity in the helical region as a result of incorporation of several positively charged residues. Consistent with the modeling results, two substitution mutants showed increased α-helical contents in the presence of 50% TFE by CD spectroscopy, relative to the parent peptide (data not shown).

All peptides were synthesized commercially by the standard solid-phase method and ordered at >95% purity. The molecular mass and purity of each synthetic peptide were further confirmed by mass spectrometry (Table IV).

### Antibacterial Activities of Fowlicidin-1 and its Analogs

Two representative Gram-negative bacteria (Escherichia coli ATCC 25922 and Salmonella enterica serovar Typhimurium ATCC 14028) and two Gram-positive bacteria (Listeria monocytogenes ATCC 19115 and Staphylococcus aureus ATCC 25923) were used to test antibacterial potency of fowlicidin-1 and its analogs by a modified broth microdilution assay as described [16, 21]. As compared with the parent peptide, the analog with deletion of three C-terminal lysines [fowlicidin-1(1-23)] or four N-terminal residues [fowlicidin-1(5-26)] or seven N-terminal residues [fowlicidin-1(8-26)] retained much of the bactericidal activity (Table V), suggesting that the cationic residues at both ends are dispensable for its antibacterial activity, but all or part of the central hydrophobic α-helical region between residues 8-23 plays a major role in killing bacteria. However, the peptide analog that is composed of entirely the central hydrophobic α-helix (residues 8-23) with a net charge of +2 became insoluble in 0.01% acetic acid and, therefore, was excluded from antibacterial assays.

TABLE V
Functional properties of fowlicidin-1 and its analogs

| | Antibacterial Activity | | | | Cytolytic Activity | | LPS Binding |
|---|---|---|---|---|---|---|---|
| | (MIC, μM) | | | | (EC50, μM) | | Activity |
| Peptide | S. aureus | Listeria | Salmonella | E. coli | Hemolytic | Cytotoxic | (EC50, μM) |
| Fowlicidin-1(1-26) | 0.5 | 2.0 | 2.0 | 4.0 | 6 | 15 | 11 |
| Fowl-1(1-15) | 13.8 | 13.8 | 3.5 | 6.9 | >443 | >443 | >443 |
| Fowl-1(1-23) | 1.1 | 2.3 | 2.3 | 4.5 | 38 | 40 | 39 |
| Fowl-1(8-26) | 2.8 | 5.6 | 2.8 | 5.6 | >360 | 159 | >260 |
| Fowl-1(5-26) | 0.6 | 2.4 | 2.4 | 4.8 | 11 | 9 | 10 |
| Fowl-1-L¹⁶ | 2.0 | 3.9 | 2.0 | 7.8 | 3 | 15 | 9 |
| Fowl-1-KLKLK | 1.9 | >7.6 | 1.9 | >7.6 | 1 | 11 | 6 |

To further examine the differential role of the N- and C-terminal short helical segments in antibacterial potency, fowlicidin-1(1-15) with omission of the C-terminal helical region after the kink at Gly¹⁶, was tested against the four bacterial strains and found to have only <2-fold reduction in minimum inhibitory concentration (MIC) toward Gram-negative bacteria, but 7- to 18-fold reduction toward Gram-positive bacteria (Table V), suggesting that the C-terminal short helix (residues 16-23) is critical in maintaining its antibacterial potency against Gram-positive but not Gram-negative bacteria. This is consistent with earlier observations that activity of cationic antimicrobial peptides against Gram-negative bacteria is generally more tolerant to structural changes [10].

In contrast to expectations, two substitution mutants (fowlicidin-1-L16 and fowlicidin-1-K⁷L¹²K¹⁴L¹⁶K¹⁸) with significant improvement in helicity, amphipathicity, and/or cationicity were found to have reduced antibacterial activity, relative to the wild-type peptide (Table V), reinforcing the notion that an intricate balance, rather than a simple enhancement in those structural parameters, dictates the antibacterial potency of the α-helical antimicrobial peptides [10, 11]. It is noteworthy that all peptide analogs showed similar kinetics in killing bacteria as the full-length peptide, with maximal activities being reached 30 min after incubation with bacteria in the presence or absence of 100 mM NaCl (data not shown). It is not clear why fowlicidin-1-K⁷L¹²K¹⁴L¹⁶K¹⁸ largely maintained its potency against S. aureus and Salmonella enterica serovar Typhimurium, but failed to completely inhibit the growth of E. coli and L. monocytogenes even at the highest concentration (7.6 μM=25 μg/ml) that we tested.

### Cytotoxicity of Fowlicidin-1 and its Analogs

To map the region that is responsible for lysis of eukaryotic cells and to identify a peptide analog with reduced cytolytic activity, all deletion and substitution mutants of fowlicidin-1 were tested individually against human erythrocyte and Madin-Darby canine kidney cells (MDCK) for their toxicity as previously described [13, 16, 22]. As summarized in Table V, Fowlicidin-1 exhibited considerable toxicity toward erythrocytes and epithelial cells with 50% effective concentrations (EC₅₀) in the range of 6-15 μM. Deletion of the last three lysines [fowlicidin-1(1-23)] resulted in a modest <4-fold reduction in toxicity, while truncation of the entire C-terminal short helix [fowlicidin-1(1-15)] caused a nearly complete loss of lytic activity toward both erythrocytes and epithelial cells, indicating that the C-terminal helix (residues 16-23), but not the last three lysines, is a critical determinant of cytotoxcity.

Relative to the full-length peptide, fowlicidin-1(5-26) maintained a similar lytic activity, whereas fowlicidin-1(8-26) only caused minimal 20% lysis of human red blood cells at 360 μM, the highest concentration tested (data not shown), suggesting possible presence of another cytotoxicity determinant in the N-terminal unstructured segment between residues 5-7. Consistent with these results, a significant >10-fold reduction in killing MDCK cells was also observed with fowlicidin-1(8-26) (Table V). Because of the fact that two peptide analogs, fowlicidin-1(1-15) and fowlicidin-1(8-26) each containing one cytolytic determinant, had substantially reduced toxicity, it is likely that the two lytic sites (residues 5-7 and 16-23) act in a synergistic manner in lysis eukaryotic cells, i.e., the presence of one determinant facilitates the action of the other.

Single substitution of Gly¹⁶ for leucine (fowlicidin-1-L16) did not lead to any obvious alterations in killing eukaryotic cells (Table V). In contrast, fowlicidin-1-K⁷L¹²K¹⁴L¹⁶K¹⁸ with a nearly perfect amphipathic helix in the center showed a 6-fold increase in lysis of red blood cells, but only slightly higher lytic activity against mammalian epithelial cells (Table V). This suggested that the amphipathic helix has a stronger binding affinity and permeability toward erythrocyte membranes than to epithelial membranes, perhaps due to the difference in the lipid composition of the two host cell types.

### LPS-Binding Activity of Fowlicidin-1 and its Analogs

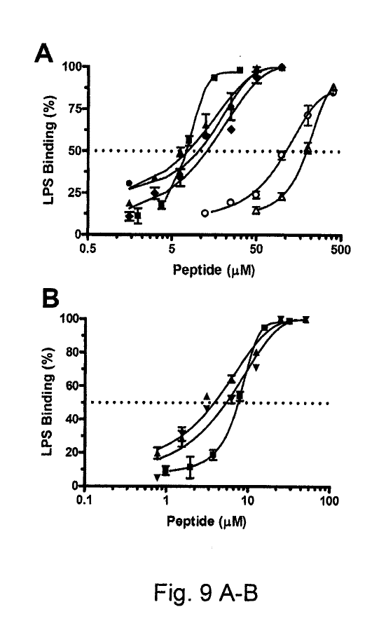

Binding and disrupting anionic LPS, the major outer membrane component of Gram-negative bacteria, is often the first step for antimicrobial peptides to interact with bacteria and permeabilize membranes [10]. Several cathelicidins, including human LL-37/hCAP-18 [21, 23], rabbit CAP-18 [24], and sheep SMAP-29 [25], have been shown to bind and neutralize LPS with EC₅₀ at low micromolar concentrations. We have also demonstrated that fowlicidin-1 has at least two LPS binding sites [16]. To map the regions involved in the binding of fowlicidin-1 to LPS, the N- and C-terminal deletion mutants were mixed with LPS, and their ability to bind LPS and inhibit LPS-mediated procoagulant activation was measured by a chromogenic Limulus amoebocyte assay [21, 25]. As shown in FIG. 9A, fowlicidin-1(1-23) and fowlicidin-1(5-26) had similar affinities for LPS to the full-length peptide, with EC₅₀ in the range of 10-39 μM (Table V), suggesting that LPS-binding sites are likely to be located in the central helical region between residues 5-23.

Residues 5-7 is clearly involved in LPS binding and may constitute the core region of one LPS-binding site, because fowlicidin-1(8-26) showed a >15-fold reduction in binding to LPS relative to fowlicidin-1(5-26), which had a similar affinity for LPS to the full-length peptide. The other LPS-binding site is likely located in the C-terminal short helix between residues 16-23, because deletion of that region [fowlicidin-1(1-15)] resulted in a >25-fold reduction in LPS binding, as compared to fowlicidin-1(1-23) FIG. 9A, Table V). It is important to note that two LPS-binding sites of fowlicidin-1 are located in the same regions where the two cytotoxicity determinants reside. This is perhaps not surprising, given that sequences that interact with anionic LPS or phospholipids on bacterial membranes are likely involved in interactions with eukaryotic cell membranes, which is a prerequisite for cytotoxicity. In fact, the hemolytic domain of SMAP-29 was also shown to overlap with a LPS-binding site at the C-terminal end [25].

To determine whether the two LPS-binding sites act in a synergistic manner, an equimolar mixture of fowlicidin-1(1-15) and fowlicidin-1(8-26) with each containing one LPS-binding site was incubated with LPS and measured for their ability to bind to LPS. As shown in FIG. 5A, the mixture displayed an enhanced affinity for LPS, approaching the full-length peptide, indicative of the synergistic nature of two LPS-binding sites. Both substitution mutants, fowlicidin-1-L16 and fowlicidin-1-K⁷L¹²K¹⁴L¹⁶K¹⁸, had minimal changes in LPS-binding affinity, relative to the native peptide, suggesting that a simultaneous enhancement in helicity, cationicity, and amphipathicity has little impact on interactions of peptides with LPS and possibly with bacterial membranes as well, which may explain why the antibacterial activities of both mutants remained largely unchanged (Table V).

### Discussion

Cathelicidins are highly conserved from birds to mammals in the prosequence, but are extremely divergent in the C-terminal mature sequence [1-3]. Cathelicidin-like molecules have also been found in the hagfish, the most ancient extant jawless fish with no adaptive immune system [26]. With the finding that fowlicidin-1 adopts an α-helix, it is now evident that at least one cathelicidin in α-helical conformation is present in each of the fish, bird, and mammalian species examined. This suggests that, in addition to the prosequence, cathelicidins appear to be conserved in the mature region structurally and presumably functionally as well. It is plausible that presence of additional structurally different cathelicidins in certain animal species may help the hosts better cope with unique microbial insults in the ecological niche where each species inhabits, given the fact that different cathelicidins appear to possess non-overlapping antimicrobial spectrum [6] and that some act synergistically in combinations in killing microbes [7]. On the other hand, innate host defense of animal species like primates and rodents containing a single cathelicidin may be compensated by the presence of a large number of other antimicrobial peptides such as a- and β-defensins [27, 28]. Conversely, pig and cattle have multiple cathelicidins but no alpha-defensins reported.

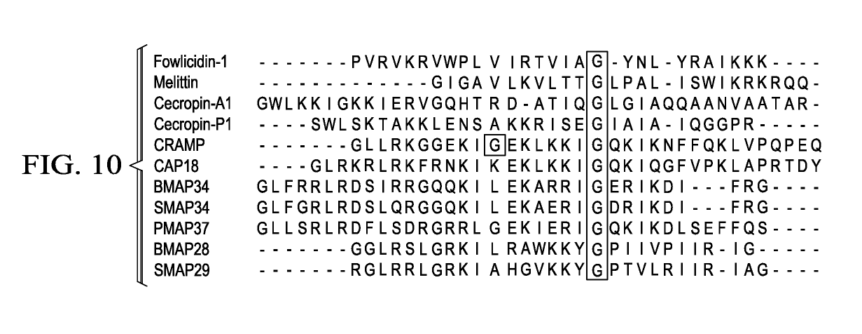

Our NMR studies revealed that, in addition to a short flexible unstructured region at the N-terminus, fowlicidin-1 is primarily composed of two short α-helical segments connected by a slight kink caused by Gly¹⁶ near the center (FIG. 7). Interestingly, such a helix-hinge-helix structural motif is not uncommon for cathelicidins. Mouse cathelicidin CRAMP [22], bovine BMAP-34 [29], and porcine PAMP-37 [30] all adopt a helix-hinge-helix structure with the hinge occurring at the central glycine (see FIG. 10, where the amino acid sequence of Fowlicidin-1 is depicted with an additional amino terminal “PV” included; this sequence is referred to as “Fowlicidin-1 (-2-26)” herein. In fact, none of the linear, naturally occurring cathelicidins are strictly α-helical. Besides peptides with helix-hinge-helix structures, a few other linear cathelicidins consist of a N-terminal helix followed by non-helical and mostly hydrophobic tails, such as rabbit CAP-18 [31], sheep SMAP-29 [25], and bovine BMAP-27 and BMAP-28 [12] (FIG. 10).

In addition to cathelicidins, a scan of over 150 helical antimicrobial peptides revealed that glycine is frequently found near the center and acts as a hinge to increase flexibility in many other protein families [10] (FIG. 10). Presence or insertion of such a hinge in the helix has been shown in many cases to be desirable, attenuating the toxicity of peptides to host cells while maintaining comparable antimicrobial potency with the peptides having no hinge sequences [10, 11]. Mutation of the hinge sequence with a helix-stabilizing residue, like leucine, will generally result in an increase in cytotoxicity and in several cases antimicrobial potency as well. However, substituting Gly16 of fowlicidin-1-L¹⁶ for leucine did not enhance antibacterial or cytolytic activity (Table V), which is likely due to the relatively low flexibility of the wild-type peptide.

A careful comparison of fowlicidin-1 with other α-helical cathelicidins indicated that the α-helix (residues 8-23) of fowlicidin-1 is much more hydrophobic and much less amphipathic than most of the mammalian cathelicidins (FIG. 10). The positive charges of fowlicidin-1 are more concentrated in the non-helical regions at both ends. Because high hydrophobicity is often associated with strong cytotoxicity [10, 11], it is perhaps not surprising to see that fowlicidin-1 is relatively more toxic than many other cathelicidins. Interestingly, fowlicidin-1 structurally more resembles melittin, a helical peptide found in honey bee venom that has a curved hydrophobic helix with positively charged residues located primarily at the C-terminal end [32] (FIG. 10). Like fowlicidin-1, melittin displays considerable antibacterial and hemolytic activities. An attempt to reduce hydrophobicity and enhance amphipathicity of the helical region of fowlicidin-1 to make fowlicidin-1-K⁷L¹²K¹⁴L¹⁶K¹⁸ led to a dramatically increased toxicity particularly toward erythrocytes with a minimum change in the antibacterial activity against certain bacteria (Table). This is consistent with an earlier conclusion that an amphipathic helix is more essential for interactions with zwitteronic lipid membranes on eukaryotic cells than for anionic lipids on prokaryotic cells [33].

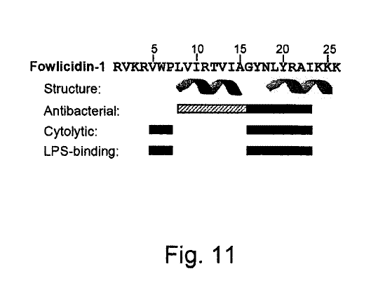

Our SAR data revealed the regions that are responsible for each of the antibacterial, LPS-binding, and cytolytic activities of fowlicidin-1 (FIG. 11). The C-terminal a-helix after the kink (residues 16-23), consisting of a stretch of eight amino acids, is required for all three functions, suggesting that this region is likely a major site for the peptide to interact with LPS and lipid membranes and to permeabilize both bacterial and eukaryotic cells. It is not surprising to see the presence of two lipophilic tyrosines (Tyr¹⁷ and Tyr²⁰) that might be critical in mediating membrane interactions for fowlicidin-1. However, the α-helix before the kink at Gly16 is likely to be involved in membrane penetration as well, because the minimum length required for a helical peptide to traverse membranes and exert antimicrobial and lytic activities is about 11-14 residues [34].

Another region, comprised of three amino acids in the N-terminal flexible region (residues 5-7), is also involved in both LPS binding and cytotoxicity, but not so important in bacterial killing (FIG. 11). It is interesting to note that among the three residues in this region is Trp6, which is known to have a preference to be inserted into lipid bilayers at the membrane-water interface [35, 36]. Because of such membrane-seeking ability, inclusion of tryptophan often renders peptides with higher affinity for membranes and more potency against bacteria [37, 38]. It is not known why tryptophan is not significantly involved in the antibacterial activity of fowlicidin-1.

It is noteworthy that the N-terminal helix of many cathelicidins plays a major role in LPS binding and bacterial killing, while the C-terminal segment is either dispensable for antimicrobial activity or more involved in cytotoxicity [12, 25, 39, 40]. However, the C-terminal helix after the kink of fowlicidin-1 is more important in killing bacteria than the N-terminal helix. Such a sharp difference in the distribution of functional domains along the peptide chain between fowlicidin-1 and other cathelicidins is probably because of a more pronounced hydrophobic nature of the helix and the presence of an additional highly flexible segment at the N-terminus of fowlicidin-1.

One aim of the present study was to identify peptide analog(s) with better therapeutic potential. Fowlicidin-1(1-23) and fowlicidin-1(5-26) had only marginal effect on either antibacterial potency or cytotoxicity, whereas fowlicidin-1(1-15) exhibited minimal toxicity up to 443 μM, but with an obvious decrease in antibacterial activity particularly against Gram-positive bacteria, implying less desirable therapeutic relevance of these peptide analogs as a broad-spectrum antibiotic. Fowlicidin-1-L16 and fowlicidin-1-K⁷L¹²K¹⁴L¹⁶K¹⁸ also had a more pronounced reduction in antibacterial activity than in toxicity, therefore with reduced clinical potential. In contrast, fowlicidin-1(8-26) with the N-terminal toxicity determinant (residues 5-7) deleted and the C-terminal antibacterial domain (residues 16-23) left unaltered, had a slight reduction in MIC against bacteria, but with >10-fold reduction in toxicity toward mammalian epithelial cells and negligible toxicity toward erythrocytes (Table V). Coupled with its smaller size, this peptide analog may represent a safer and more attractive therapeutic candidate than the parent peptide. Given the fact that fowlicidin-1 is broadly effective against several common bacterial strains implicated in cystic fibrosis, including S. aureus, Klebsiella pneumoniae, and Pseudomonas aeruginosa in a salt-independent manner [16], its analog, fowlicidin-1(8-26), should be useful in controlling chronic respiratory infections of cystic fibrosis patients. These results also suggested the usefulness of systematic SAR studies in improving the safety and target specificity of antimicrobial peptides.

### References for Example 2

1. Zanetti M (2004) Cathelicidins, multifunctional peptides of the innate immunity. J Leukoc Biol 75, 39-48.
2. Zaiou M & Gallo R L (2002) Cathelicidins, essential gene-encoded mammalian antibiotics. J Mol Med 80, 549-561.
3. Lehrer R I & Ganz T (2002) Cathelicidins: a family of endogenous antimicrobial peptides. Curr Opin Hematol 9, 18-22.
4. Zasloff M (2002) Antimicrobial peptides of multicellular organisms. Nature 415, 389-395.
5. Hancock RE & Patrzykat A (2002) Clinical development of cationic antimicrobial peptides: from natural to novel antibiotics. Curr Drug Targets Infect Disord 2, 79-83.
6. Zanetti M, Gennaro R, Skerlavaj B, Tomasinsig L & Cireo R (2002) Cathelicidin peptides as candidates for a novel class of antimicrobials. Curr Pharm Des 8, 779-793.
7. Yan H & Hancock R E (2001) Synergistic interactions between mammalian antimicrobial defense peptides. Antimicrob Agents Chemother 45, 1558-1560.
8. Levy O, Ooi C E, Weiss J, Lehrer R I & Elsbach P (1994) Individual and synergistic effects of rabbit granulocyte proteins on Escherichia coli. J Clin Invest 94, 672-682.
9. Nagaoka I, Hirota S, Yomogida S, Ohwada A & Hirata M (2000) Synergistic actions of antibacterial neutrophil defensins and cathelicidins. Inflamm Res 49, 73-79.
10. Tossi A, Sandri L & Giangaspero A (2000) Amphipathic, alpha-helical antimicrobial peptides. Biopolymers 55, 4-30.
11. Dathe M & Wieprecht T (1999) Structural features of helical antimicrobial peptides: their potential to modulate activity on model membranes and biological cells. Biochim Biophys Acta 1462, 71-87.
12. Skerlavaj B, Gennaro R, Bagella L, Merluzzi L, Risso A & Zanetti M (1996) Biological characterization of two novel cathelicidin-derived peptides and identification of structural requirements for their antimicrobial and cell lytic activities. J Biol Chem 271, 28375-28381.
13. Shin S Y, Park E J, Yang S T, Jung H J, Eom S H, Song W K, Kim Y, Hahm K S & Kim J I (2001) Structure-activity analysis of SMAP-29, a sheep leukocytes-derived antimicrobial peptide. Biochem Biophys Res Commun 285, 1046-1051.
14. van Dijk A, Veldhuizen E J, van Asten A J & Haagsman H P (2005) CMAP27, a novel chicken cathelicidin-like antimicrobial protein. Vet Immunol Immunopathol 106, 321-327.
15. Lynn D J, Higgs R, Gaines S, Tierney J, James T, Lloyd A T, Fares M A, Mulcahy G & O'Farrelly C (2004) Bioinformatic discovery and initial characterisation of nine novel antimicrobial peptide genes in the chicken. Immunogenetics 56, 170-177.
16. See Example 1.
17. Wuthrich K (1986) NMR of Proteins and Nucleic Acids. John Wiley and Sons Inc., New York.
18. Wishart D S, Sykes B D & Richards F M (1992) The chemical shift index: a fast and simple method for the assignment of protein secondary structure through NMR spectroscopy. Biochemistry 31, 1647-51.
19. Laskowski R A, Rullmannn J A, MacArthur M W, Kaptein R & Thornton J M (1996) AQUA and PROCHECK-NMR: programs for checking the quality of protein structures solved by NMR. J Biomol NMR 8, 477-486.
20. Fiser A & Sali A (2003) Modeller: generation and refinement of homology-based protein structure models. Methods Enzymol 374, 461-491.
21. Turner J, Cho Y, Dinh N N, Waring A J & Lehrer R I (1998) Activities of LL-37, a cathelin-associated antimicrobial peptide of human neutrophils. Antimicrob Agents Chemother 42, 2206-2214.
22. Yu K, Park K, Kang S W, Shin S Y, Hahm K S & Kim Y (2002) Solution structure of a cathelicidin-derived antimicrobial peptide, CRAMP as determined by NMR spectroscopy. J Pept Res 60, 1-9.
23. Larrick J W, Hirata M, Balint R F, Lee J, Zhong J & Wright S C (1995) Human CAP18: a novel antimicrobial lipopolysaccharide-binding protein. Infect Immun 63, 1291-1297.
24. Larrick J W, Hirata M, Zheng H, Zhong J, Bolin D, Cavaillon J M, Warren H S & Wright S C (1994) A novel granulocyte-derived peptide with lipopolysaccharide-neutralizing activity. J Immunol 152, 231-240.
25. Tack B F, Sawai M V, Kearney W R, Robertson A D, Sherman M A, Wang W, Hong T, Boo L M, Wu H, Waring A J & Lehrer R I (2002) SMAP-29 has two LPS-binding sites and a central hinge. Eur J Biochem 269, 1181-1189.
26. Uzzell T, Stolzenberg E D, Shinnar A E & Zasloff M (2003) Hagfish intestinal antimicrobial peptides are ancient cathelicidins. Peptides 24, 1655-1667.
27. Patil A, Hughes A L & Zhang G (2004) Rapid evolution and diversification of mammalian alpha-defensins as revealed by comparative analysis of rodent and primate genes. Physiol Genomics 20, 1-11.
28. Patil A A, Cai Y, Sang Y, Blecha F & Zhang G (2005) Cross-species analysis of the mammalian beta-defensin gene family: presence of syntenic gene clusters and preferential expression in the male reproductive tract. Physiol Genomics 23, 5-17.
29. Gennaro R, Scocchi M, Merluzzi L & Zanetti M (1998) Biological characterization of a novel mammalian antimicrobial peptide. Biochim Biophys Acta 1425, 361-368.
30. Tossi A, Scocchi M, Zanetti M, Storici P & Gennaro R (1995) PMAP-37, a novel antibacterial peptide from pig myeloid cells. cDNA cloning, chemical synthesis and activity. Eur J Biochem 228, 941-946.
31. Chen C, Brock R, Luh F, Chou P J, Larrick J W, Huang R F & Huang T H (1995) The solution structure of the active domain of CAP18—a lipopolysaccharide binding protein from rabbit leukocytes. FEBS Lett 370, 46-52.
32. Terwilliger T C, Weissman L & Eisenberg D (1982) The structure of melittin in the form I crystals and its implication for melittin's lytic and surface activities. Biophys J 37, 353-361.
33. Dathe M, Schumann M, Wieprecht T, Winkler A, Beyermann M, Krause E, Matsuzaki K, Murase O & Bienert M (1996) Peptide helicity and membrane surface charge modulate the balance of electrostatic and hydrophobic interactions with lipid bilayers and biological membranes. Biochemistry 35, 12612-22.
34. Blondelle S E & Houghten R A (1992) Design of model amphipathic peptides having potent antimicrobial activities. Biochemistry 31, 12688-12694.
35. Yau W M, Wimley W C, Gawrisch K & White S H (1998) The preference of tryptophan for membrane interfaces. Biochemistry 37, 14713-14718.
36. Cook G A, Prakash O, Zhang K, Shank L P, Takeguchi W A, Robbins A, Gong Y X, Iwamoto T, Schultz B D & Tomich J M (2004) Activity and structural comparisons of solution associating and monomeric channel-forming peptides derived from the glycine receptor m2 segment. Biophys J 86, 1424-1435.
37. Deslouches B, Phadke S M, Lazarevic V, Cascio M, Islam K, Montelaro R C & Mietzner T A (2005) De novo generation of cationic antimicrobial peptides: influence of length and tryptophan substitution on antimicrobial activity. Antimicrob Agents Chemother 49, 316-322.
38. Subbalakshmi C, Bikshapathy E, Sitaram N & Nagaraj R (2000) Antibacterial and hemolytic activities of single tryptophan analogs of indolicidin. Biochem Biophys Res Commun 274, 714-716.
39. Larrick J W, Hirata M, Shimomoura Y, Yoshida M, Zheng H, Zhong J & Wright S C (1993) Antimicrobial activity of rabbit CAP18-derived peptides. Antimicrob Agents Chemother 37, 2534-2539.
40. Tossi A, Scocchi M, Skerlavaj B & Gennaro R (1994) Identification and characterization of a primary antibacterial domain in CAP18, a lipopolysaccharide binding protein from rabbit leukocytes. FEBS Lett 339, 108-112.
41. Soulages J L, Arrese E L, Chetty P S & Rodriguez V (2001) Essential role of the conformational flexibility of helices 1 and 5 on the lipid binding activity of apolipophorin-III. J Biol Chem 276, 34162-34166.
42. Sreerama N, Venyaminov S Y & Woody R W (2000) Estimation of protein secondary structure from circular dichroism spectra: inclusion of denatured proteins with native proteins in the analysis. Anal Biochem 287, 243-251.
43. Dhanasekaran M, Baures P W, VanCompernolle S, Todd S & Prakash O (2003) Structural characterization of peptide fragments from hCD81-LEL. J Pept Res 61, 80-89.
44. Yu X Q, Prakash O & Kanost M R (1999) Structure of a paralytic peptide from an insect, Manduca sexta. J Pept Res 54, 256-261.
45. Wuthrich K, Billeter M & Braun W (1983) Pseudo-structures for the 20 common amino acids for use in studies of protein conformations by measurements of intramolecular proton-proton distance constraints with nuclear magnetic resonance. J Mol Biol 169, 949-961.
46. Clore G M, Gronenborn A M, Nilges M & Ryan C A (1987) Three-dimensional structure of potato carboxypeptidase inhibitor in solution. A study using nuclear magnetic resonance, distance geometry, and restrained molecular dynamics. Biochemistry 26, 8012-8023.
47. Brunger A T, Adams P D, Clore G M, DeLano W L, Gros P, Grosse-Kunstleve R W, Jiang J S, Kuszewski J, Nilges M, Pannu N S, Read R J, Rice L M, Simonson T & Warren G L (1998) Crystallography & NMR system: A new software suite for macromolecular structure determination. Acta Crystallogr D Biol Crystallogr 54, 905-921.
48. Koradi R, Billeter M & Wuthrich K (1996) MOLMOL: a program for display and analysis of macromolecular structures. J Mol Graph 14, 51-55.

### Example 3

### The Central Kink Region of α-Helical Fowlicidin-2 is Critically Involved in Antibacterial and Lipopolysaccharide-Neutralizing Activities

With rapid emergence of antibiotic-resistant pathogens, there is an unprecedented urgency to develop new antimicrobials with a less likelihood of gaining resistance. Cationic antimicrobial peptides, an integral component of innate immunity found in virtually all species of life, are capable of killing a variety of pathogens with a similar efficiency against the strains that are resistant to conventional antibiotics (1,2). Because of nonspecific membrane-lytic activities of most cationic peptides, it is conceivable that bacteria are much more difficult to develop resistance against these peptides than conventional antibiotics (2-4). Therefore, these peptides are being actively explored as a new class of antimicrobials. However, there are several major barriers that impede the development of cationic peptides as therapeutic agents. Factors like salt and serum are known to suppress microbicidal activities of a majority of these peptides (5-7). The cytotoxicity associated with many peptides is also noticeable. Furthermore, like any peptide-based drugs, proneness to degradation by endogenous proteases is also of particular concern. Various strategies have been developed in attempt to reduce the side-effects and enhance antimicrobial efficacy and serum stability of these peptides. To date, a number of structure-activity relationship studies have lead to identification of novel peptide analogs with better therapeutic potential by altering certain physico-chemical and structural properties, such as charge, amphipathicity, hydrophobicity, helicity, and cyclization (8-10).

Cathelicidins, a major family of vertebrate antimicrobial peptides, are receiving a particular attention as novel antimicrobials, due to simple structures (devoid of cysteines in most cases) and high antibacterial efficacy relative to other families of cationic peptides (11-14). In addition to a broad spectrum of antimicrobial activities, many cathelicidins have capacity to directly bind lipopolysaccharide (LPS)4 and lipoteichoic acid in vitro and neutralize Gram-negative and Gram-positive bacteria-induced septic shock in vivo (15-17). Thus these cathelicidins may also represent excellent therapeutic candidates for sepsis treatment.

Example 1 describes the identification of three novel chicken cathelicidins, namely fowlicidins 1-3, with highly potent antibacterial and LPS-neutralizing activities (18). In comparison with fowlicidin-1, fowlicidin-2 exhibited similar antibacterial efficacy with slightly less lytic activity to host cells (18). To further reduce the toxicity of fowlicidin-2, in this report we first determined the tertiary structure of fowlicidin-2 by nuclear magnetic resonance (NMR) spectroscopy. Based on the structural information, several deletion analogs were further designed and analyzed for their antibacterial, cytotoxic, membrane-permeabilizing, and LPS-neutralizing activities. We demonstrated that four amino acids in the central kink region (residues 15-18) are critically important in peptide-membrane interactions as well as in bacterial killing and LPS neutralization. We further identified that two short peptide analogs containing either the N- or C-terminal helical segment together with residue 15-18 retain substantial antibacterial and LPS-neutralizing activities but with a significant reduction in cytotoxicity, relative to the parent peptide.

### Materials and Methods

Peptide synthesis—Fowlicidin-2 was synthesized using the standard solid-phase method by SynPep (Dublin, Calif.) and its analogs by Bio-Synthesis (Lewisville, Tex.) (Table VI). The peptides were purified through reverse phase-HPLC and ordered at >95% purity. The mass and purity of each peptide were further confirmed by MALDI-TOF mass spectrometry using the Voyager DE-PRO instrument (Applied Biosystems, Foster City, Calif.) housed in the Recombinant DNA/Protein Resource Facility of Oklahoma State University. The average mass of each peptide determined by mass spectrometry was consistent with the mass calculated from the sequence (Table VI).

TABLE VI
Fowlicidin-2 and its analogs

| | | | | Mass | |
|---|---|---|---|---|---|
| Peptide | Sequence | Charge | Length | Calculated | Observed |

| | | | | | |
|---|---|---|---|---|---|
| Fowl-2(1-31) | LVQRGRFGRFLRKIR | +10 | 31 | 3761.5 | 3760.6 |
| | RFRPKVTITIQGSARF | | | | |
| | (SEQ ID NO: 40) | | | | |
| Fowl-2(1-14) | LVQRGRFGRFLRKI | +5 | 14 | 1746.1 | 1746.1 |
| | (SEQ ID NO: 41) | | | | |
| Fowl-2(1-15) | LVQRGRFGRFLRKIR | +6 | 15 | 1902.3 | 1903.2 |
| | (SEQ ID NO: 42) | | | | |
| Fowl-2(1-18) | LVQRGRFGRFLRKIR | +8 | 18 | 2361.9 | 2363.0 |
| | RFR | | | | |
| | (SEQ ID NO: 43) | | | | |
| Fowl-2(15-31) | RRFRPKVTITIQGSARF | +5 | 17 | 2033.4 | 2034.0 |
| | (SEQ ID NO: 44) | | | | |
| Fowl-2(19-31) | PKVTITIQGSARF | +2 | 13 | 1417.7 | 1417.8 |
| | (SEQ ID NO: 45) | | | | |

CD spectroscopy—To determine the secondary structure of fowlicidin-2 and its analogs, circular dichroism (CD) spectroscopy was performed with a Jasco-715 spectropolarimeter using a 0.1-cm path length cell over the 180-260 nm range as we previously described (19). The spectra were acquired at 25° C. every 1 nm with a 2-s averaging time per point and a 1-nm band pass. Peptides (10 μM) were measured in 50 mM potassium phosphate buffer, pH 7.4, with or without different concentrations of trifluoroethanol (TFE) (0%, 10%, 20%, 40%, and 60%) or SDS micelles (0.25% and 0.5%). Mean residue ellipticity (MRE) was expressed as [θ]_{MRE} (deg.cm².dmo⁻¹). The contents of six types of the secondary structural elements, including regular and distorted α-helix, regular and distorted β-sheet, turns, and unordered structures, were analyzed with the program SELCON3 (20).
NMR spectroscopy—To determine the solution structure of fowlicidin-2, 1D and 2D ¹H NMR experiments were performed with an 11.75 T Varian UNITYplus spectrometer (Varian, Palo Alto, Calif., USA), operating at 499.96 MHz, with a 3-mm triple-resonance inverse detection probe. Fowlicidin-2 (3 mM) was used to record TOCSY and NOESY experiments at 25° C. in 50% deuterated TFE (TFE-d3)/50% H₂O essentially as described (19). A series of 1D experiments were conducted from 5-35° C. with 5° C. increments. Water peak suppression was obtained by low-power irradiation of the H2O resonance. A total of 256 increments of 4K data points were recorded for the 2D experiments. All data sets were obtained in the hypercomplex phase-sensitive mode. TOCSY spectra were recorded by using MLEV-17 for isotropic mixing for 100 ms at a B1 field strength of 8 KHz. NOESY experiments were performed with mixing times of 100, 300, and 400 ms, and the residual TFE methylene peak was considered a reference for the chemical shift values. Varian software, VNMR 6.1C, on a Silicon Graphics Octane workstation was used for data processing, and Sparky 3 software (Goddard, T. D. and D. G. Kneller, University of California, San Francisco, Calif.) was used for data analysis.
Structure calculation—Proton resonance assignments were made using 2D TOCSY for intra-residue spin systems and NOESY spectra for inter-residue connectivities as described (21). A total of 174 distance constraints obtained from the NOESY spectrum were used for structure calculations. NOE peak were classified as intra-residue, sequential, medium, and long ranges. NOE cross peak intensities were classified as strong (1.8-2.7 Å), medium (1.8-3.5 Å), weak (1.8-4.0 Å), and very weak (1.8-5.0 Å). Upper distance limits for NOEs involving methyl protons and non-stereospecifically assigned methylene protons were corrected appropriately by adding 1 Å to the constraints for center averaging (22). The obtained distance restraints were then used to create initial peptide structures starting from extended structures using the program CNS version 1.1 (23), which uses both a simulated annealing protocol and molecular dynamics to produce low energy structures with minimum distance and geometry violations. Default parameters were used to generate 100 structures for the initial CNS run. A second round of calculations generated 20 structures, from which 10 with fewer or no restriction violations and the lowest energy were selected and analyzed with Sybyl 7.1 (Tripos, St. Louis, Mo.). Surface accessibilities of fowlicidin-2 was generated using GRASP (24).
Antibacterial assay—Three representative Gram-negative bacteria (Escherichia coli ATCC 25922 and Salmonella enterica serovar Typhimurium ATCC 14028, and Klebsiella pneumoniae ATCC 13883) and four Gram-positive bacteria (Listeria monocytogenes ATCC 19115, Staphylococcus aureus ATCC 25923, and two methicilin-resistant S. aureus ATCC BAA-39 and S. aureus ATCC 43300) were purchased from ATCC (Manassas, Va.) or MicroBiologics (St. Cloud, Minn.). The antibacterial activities of fowlicidin-2 and its analogs were tested by using a modified broth microdilution assay in the bicarbonate buffer as described (25). Briefly, bacteria were cultured overnight in tryptic soy broth (TSB) at 37° C., diluted 1:1000 in fresh subculture medium containing 20% TSB, 10% fetal bovine serum (FBS), 100 mM NaCl, 25 mM NaHCO₃, and 1 mM NaH₂PO₄, and grown to the mid-log phase. Cells were then washed twice with 10 mM sodium phosphate buffer, pH 7.4, and resuspended to 5×10⁵ colony-forming units (CFU)/ml in the assay medium containing 20% TSB, 25 mM NaHCO₃, and 1 mM NaH₂PO₄ (25). If necessary, 100 mM NaCl was incorporated to test the effect of salinity on antibacterial activity. Bacteria (90 μl) were dispended into 96-well plates followed by addition in duplicate of 10 μl of peptides in serial two-fold dilutions in 0.01% acetic acid. The minimum inhibitory concentration (MIC) of each peptide against each bacterial strain was determined as the lowest concentration that gave no visible bacterial growth after overnight incubation at 37° C.
Effect of serum on the antibacterial activity—The influence of serum on the antibacterial activity of fowlicidin-2 and its analogs was evaluated by the radial diffusion assay as described (26,27). Briefly, S. aureus ATCC 25923 was grown in TSB to the mid-log phase, and washed twice with 10 mM sodium phosphate buffer, pH 7.4, and then mixed evenly to 4×10⁵ CFU/ml with the underlay gel containing 10 mM sodium phosphate buffer (pH 7.4), 0.3 mg/ml of TSB powder, and 1% (w/v) agarose. Sample wells (3 mm in diameter) were punched in the underlay gel. Peptides (0.5 or 1 μg), diluted in 4 μl of 0.01% acetic acid containing 0, 20% and 50% serum, were added to each well. After 3 h incubation at 37° C., the overlay gel containing 6% TSB powder, 1% agarose, and 10 mM sodium phosphate buffer (pH 7.4) was poured onto the underlay gel and incubated overnight at 37° C. to visualize the bacterial clearance zones.
Inner membrane permeabilization assay—The kinetics of permeabilization of bacterial cytoplasmic membranes by fowlicidin-2 and its analogs were determined using E. coli ML-35p similarly as described (25). E. coli ML-35p, which constitutively expresses β-galactosidase in the cytosol but lacks lactose permease, was cultured overnight in TSB at 37° C., diluted 1:1000 and grown for additional 3 h at 37° C. Cells were washed twice with 10 mM sodium phosphate buffer, pH 7.4 and diluted to A₆₀₀=0.03 (equivalent to 2.5-5×10⁷ CFU/ml) in the same buffer containing 1% TSB with and without 100 mM NaCl. Bacteria (80 μl) were then dispended into a 96-well plate, followed by addition of each peptide (at 0.25, 0.5 or 1 MIC concentration) and 1.5 mM of a chromogenic substrate of β-galactosidase, p-nitrophenyl-β-D-galactopyranoside (ONPG) (Sigma, St. Louis, Mo.) to a total volume of 100 μ. Because the MIC of fowlicidin-2(19-31) could not be determined even at the highest peptide concentration tested (>32 μM) by the modified broth microdilution method described above, the concentration of this analog used was 16 μM. The plate was incubated at 37° C. for 2 h with shaking and reading every two minutes for monitoring the production of o-nitrophenol at 420 nm. Negative controls contained no peptide but an equal volume of 0.01% acetic acid.
Hemolytic assay—The hemolytic activity of fowlicidin-2 and its analogs were determined as described in Examples 1 and 2.
Cytotoxicity assay—The toxicity of fowlicidin-2 and its analogs was evaluated with human colonic epithelial Caco-2 cells (ATCC) by using alamarBlue dye (Biosource) similarly as described in Examples 1 and 2.
Modulation of LPS-induced proinflammatory gene expression—The effect of fowlicidin-2 and its analogs on the expression of LPS-induced proinflammatory genes were evaluated as described in Example 1.

### Results

Solution structure of fowlicidin-2—The secondary structure of fowlicidin-2 was determined by CD spectroscopy in different concentrations of TFE and SDS as described in Example 2. Fowlicidin-2 was largely unstructured in phosphate buffer, but the α-helical content was dose-dependently increased in the presence of TFE. The α-helicity rose from 3% to 36% as the TFE concentration increased from 10% to 60%. A similar trend was also observed in 0.25% and 0.5% SDS micelles (data not shown).

Two-dimensional 1H NMR spectroscopy was next performed to determine the tertiary structure of fowlicidin-2 in TFE:H2O (1:1, v/v) similarly as described in Example 2. Sequence-specific resonance assignments were obtained by using the TOCSY, and NOESY spectra. The TOCSY spectra acquired at 25° C. gave good chemical shift dispersion with limited spectral overlap, enabling the assignment of the spin systems for all residues. A spatial distance constraint was further assigned from each NOE specific peak in the NOESY spectra. A number of nonsequential d_{αN(i, i+3)} NOES that are clearly indicative of α-helical conformation were observed from Gly⁵ to Phe and from Val²¹ to Arg³⁰. The characteristic chemical shift index of these amino acids also confirmed the helical conformation in these two regions.

For structural calculations, a total of 174 NOE distance constraints involving 74 inter-residue, 53 sequential, and 37 medium range constraints were used. From 100 conformers calculated, 10 structures with the lowest energy were retained for further analysis. A Ramachandran plot, produced by PROCHECK-NMR (28), showed that 64.5% residues are in the most favored region and 32.2% in additional allowed regions, respectively. A superimposition of the 10 lowest energy structures showed a considerable degree of flexibility with a pairwise root mean square deviation (rmsd) of backbone of 3.86 Å. However, alignments along residues 6-12 and 23-27 of the 10 lowest energy structures showed the backbone rmsd values of 1.55 and 0.28 Å, suggesting the two α-helical segments are relatively rigid, whereas other regions are flexible.

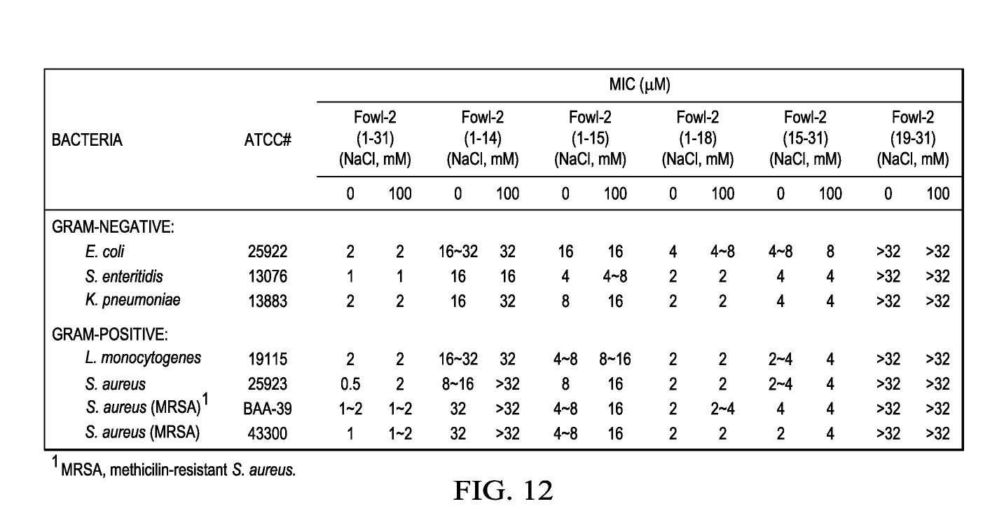

The average solution structure of fowlicidin-2 indicated that it has two well-defined α-helices from Arg⁶ to Arg¹² and from Ile²³ to Gly²⁷, a bend from Phe17 to Lys²⁰, and a flexible region at the N-terminus from Leu¹ to Arg⁴. In contrast to most α-helical cathelicidins with a limited degree of kink near the center (19,29,30), the bending of fowlicidin-2 in the central region is excessive, due to the presence of Pro¹⁹. A closer examination revealed that the N-terminal α-helix adopts a typical amphipathic structure, while the C-terminal α-helix is highly hydrophobic. It is noted that the central kink region of fowlicidin-2 is highly positively charged, containing six cationic residues.
Design and confirmation of fowlicidin-2 analogs—To probe the impact of two short helical segments and the central kink region on antibacterial, cytotoxic, and LPS-binding activities of fowlicidin-2, several analogs with deletion of either N- or C-terminal residues were designed (Table VI), synthesized chemically, and purified to >95% homogeneity by reverse phase-HPLC. The mass and purity of each peptide were further confirmed by mass spectrometry (Table VI). The secondary structure of each peptide was determined by CD spectroscopy in the presence of 60% TFE (data not shown) or 0.5% SDS (data not shown). CD spectra showed that all peptide analogs have a higher degree of α-helicity than the parent peptide. Three C-terminal deletion analogs, namely fowlicidin-2(1-14), fowlicidin-2(1-15) and fowlicidin-2(1-18), have a gradual increase in the α-helical content ranging from 44% and 53% to 62%, suggesting that residues 15-18 in the central kink region of the parent peptide likely adopt an α-helical conformation without Pro19. The α-helical content of two N-terminal deletion analogs, fowlicidin-2(15-31) and fowlicidin-2(19-31), were estimated to be 42% and 79%, respectively, consistent with the secondary structural contents predicted from the parent peptide.
Antibacterial activities of fowlicidin-2 and its analogs—A modified broth microdilution assay was used to test the antibacterial activity of fowlicidin-2 and its analogs using a bicarbonate-based buffer as described (25). Three representative Gram-negative bacteria (E. coli ATCC 25922 and S. enterica serovar Typhimurium ATCC 14028, and K. pneumoniae ATCC 13883) and four Gram-positive bacteria (L. monocytogenes ATCC 19115, S. aureus ATCC 25923, and two methicilin-resistant strains of S. aureus ATCC BAA-39 and ATCC 43300) were included in the assay. As shown in tabular form in FIG. 12, fowlicidin-2 is potent against all bacterial strains with the MIC values in the range of 0.5-2 μM, largely insensitive to salt. In contrast, the analog containing only the N-terminal α-helix [fowlicidin-2(1-14)] exhibited=8-fold reduction in antibacterial activity. The analog consisting solely of the C-terminal α-helix [fowlicidin-2(19-31)] failed to kill bacteria even at 32 μM, the highest concentration tested. These results suggested neither α-helical segments alone are sufficient in maintaining the antibacterial activity of the parent peptide.

As compared with fowlicidin-2(1-14), fowlicidin-2(1-15) and (1-18) with inclusion of additional positively charged residues in the central bending region of the parent peptide showed an graduate increase in antibacterial potency (FIG. 12), consistent with earlier observations that a simultaneous increase in cationicity and α-helicity is often directly correlated with the antibacterial efficacy (9,10). Fowlicidin-2(1-18) with a concurrent increase of +3 in the net charge and 18% in the α-helical content exhibited 8- to 16-fold enhancement in bactericidal efficiency, as compared with fowlicidin-2(1-14). In fact, the MIC values of fowlicidin-2(1-18) were approaching to those of the parent peptide, with only =2-fold difference against all bacteria tested (FIG. 12). These results demonstrated the critical involvement of the four amino acids in the bending region from Arg¹⁵ to Arg¹⁸ in killing bacteria.

Relative to fowlicidin-2(19-31), fowlicidin-2(15-31) containing additional residues from Arg¹⁵ to Arg¹⁸ also demonstrated significant antibacterial activities, with only 2- to 4-fold reduction in MIC as compared with the parent peptide. These results further confirmed that the 4-amino acid segment from Arg¹⁵ to Arg¹⁸ in the central kink region is of vital importance in maintenance of the bactericidal activity of fowlicidin-2.

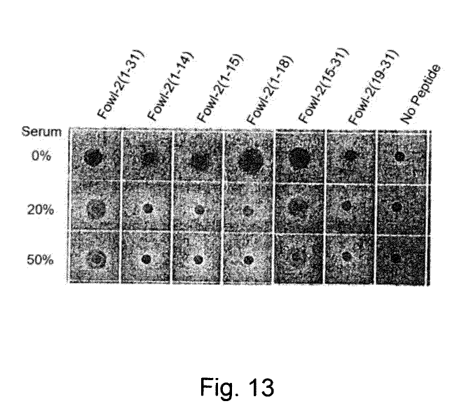

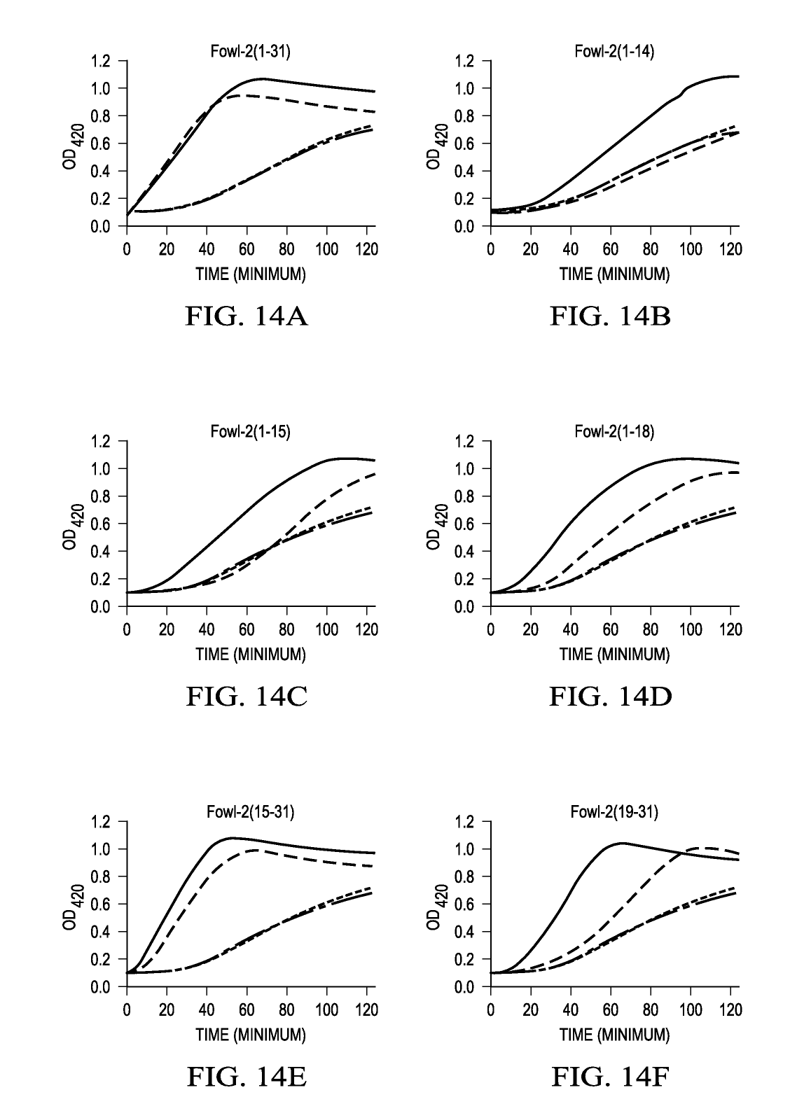

Many antimicrobial peptides show a reduced antimicrobial activity in the presence of serum or physiological concentrations of NaCl (5-7,31). However, none of the fowlicidin-2 analogs displayed >2-fold reduction in MIC with addition of 100 mM NaCl in the assay buffer (FIG. 12). It is particularly true with fowlicidin-2(1-18) and fowlicidin-2(15-31) whose antibacterial activity were largely unaffected by salt. The effect of serum on the antibacterial potency of peptides were also tested against S. aureus ATCC 29523 by the radial diffusion assay (26,27) using 0.5 μg/well of each peptide in the presence and absence of 20% or 50% human serum. As shown in FIG. 13, serum did not affect the antibacterial activity of the parent peptide or fowlicidin-2(15-31), but inhibited the activities of three C-terminal deletion analogs, namely fowlicidin-2(1-14), fowlicidin-2(1-15) and fowlicidin-2(1-18). However, when used at 1 μg/well, all peptides including three C-terminal deletion analogs maintained the antibacterial potency in 20% and 50% serum (data not shown), implying that high concentration might alter the peptide confirmation and/or cause peptide oligomerization, which may collectively resist the inhibition or cleavage by certain serum proteins.
Permeabilization of bacterial cytoplasmic membrane by fowlicidin-2 and its analogs—Electrostatic interaction with and subsequent disruption of bacterial membrane is a common mode of action for most antimicrobial peptides (2-4). The ability of fowlicidin-2 and its analogs to permeabilize the inner membranes of bacteria using 0.5 MIC of each peptide and an E. coli strain ML-35p with constitutive expression of cytosolic β-galactosidase as described (25). The disruption of E. coli ML-35p inner membrane results in the release of β-galactosidase, which in turn hydrolyzes ONPG, a chromogenic substrate to generate a color product, o-nitrophenol, which can be monitored for absorbance at 420-450 nm. As shown in FIG. 14, full-length fowlicidin-2 permeabilized the inner membrane of E. coli quickly in a time-dependent fashion with lysis occurring almost immediately upon contact of the peptide with bacteria and the peak level being reached within 50-60 min, consistent with our earlier time-kill assay that fowlicidin-2 achieved maximum bacterial killing within 30-60 min of incubation (Example 1). These results also suggested that physical membrane disruption is the major mechanism of bacterial killing by fowlicidin-2. In agreement with the broth microdilution assay (FIG. 12) and the radial diffusion assay (FIG. 13), salt did not affect the killing kinetics of fowlicidin-2 (FIG. 14).

All three C-terminal deletion analogs at 0.5 MIC concentrations reached peak lysis within 2 h in the absence of salt (FIG. 14), indicating that the N-terminal α-helical segment alone can result in membrane permeabilization, although at a very slow rate. In fact, relative to the parent peptide, the onset of lysis did not occur until ˜20 min following peptide-bacterial incubation for fowlicidin-2(1-14), 10-15 min for fowlicidin-2(1-15), and ˜5 min for fowlicidin-2(1-18) (FIG. 14). Peak lysis also delayed by 50-60 min for fowlicidin-2(1-14) and fowlicidin-2(1-15), and by 10-20 min for fowlicidin-2(1-18) in the absence of salt. Presence of salt further delayed membrane permeabilization for all three analogs. Although fowlicidin-2(1-18) reached the maximum lysis at 120 min, fowlicidin-2(1-14) remained largely inactive within 2 h, suggesting that the four-amino acid segment from Arg¹⁵ to Arg¹⁸ appears to be important in resisting the adverse effects of salt on peptide-membrane interactions. A comparison among three C-terminal deletion analogs also suggested that cationicity and helicity are positively correlated with the kinetics of membrane interaction and permeabilization with and without salt.

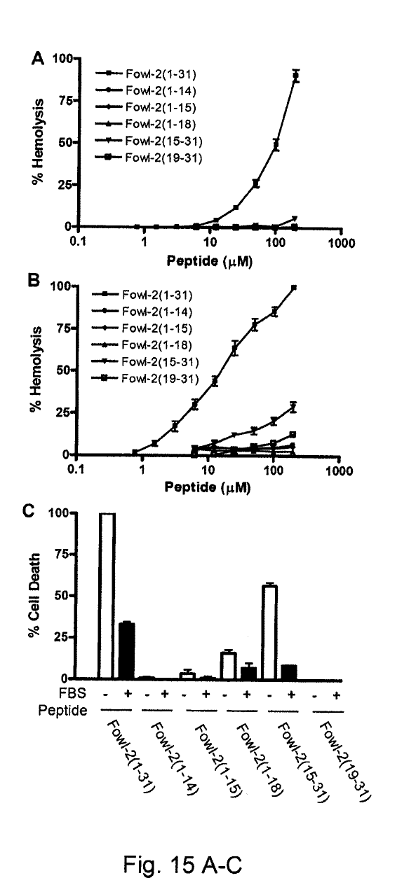

Two N-terminal deletion analogs, fowlicidin-2(15-31) and fowlicidin-2(19-31), also showed time-dependent membrane lysis of E. coli (FIG. 14), indicating that, similar to the N-terminal helix, the C-terminal helix alone also appears to be capable of interacting and permeabilizing bacterial membrane. It is interesting to note that, however, fowlicidin-2(19-31) at 16 μM did not lead to an appreciable level of bacterial killing FIG. 12), implying that, membrane permeabilization and bacterial killing may not necessarily to be directly related. The fact that fowlicidin-2(19-31) approached to the peak level of lysis at 50-60 min in the absence of salt and at 90-100 min in the presence of 100 mM NaCl (FIG. 14), indicating that the C-terminal helix may be much more efficient in lysis of membranes than the N-terminal helix. Fowlicidin-2(15-31) with inclusion of the four-amino acid segment from Arg¹⁵ to Arg¹⁸ further enhanced the kinetics of membrane lysis, and furthermore, such membrane permeabilizing activity was not affected by salt. Collectively, these observations also confirmed that the central kink region, particularly from Arg¹⁵ to Arg¹⁸, is critical in mediating salt-resistant interactions between peptides and bacterial membranes.
Cytotoxicity of fowlicidin-2 and its analogs—To identify the region(s) that are involved in cytotoxicity and possibly the analog(s) with reduced toxicity, fowlicidin-2 and its analogs were tested for their toxicities to human erythrocytes and colonic epithelial Caco-2 cells as previously described (Example 1). The full-length fowlicidin-2 peptide displayed a noticeable, dose-dependent hemolytic activity. At 200 μM, fowlicidin-2 lysed 80% and 100% erythrocytes in the presence (FIG. 15A) and absence of 10% FBS (FIG. 15B), respectively. Remarkably, all three C-terminal deletion analogs and fowlicidin-2(19-31) showed essentially no hemolysis even at 200 regardless of serum (FIGS. 15A and 15B). Fowlicidin-2(15-31) also lost hemolytic activity in the presence of serum (FIG. 15A), and only exhibited 30% of lysis in the absence of serum (FIG. 6B), when applied at 200 μM.

As for Caco-2 cells, the full-length peptide showed 100% killing in the presence of 10% FBS and 30% killing without FBS, whereas all analogs lost their toxicity significantly. Fowlicidin-2(1-14), fowlicidin-2(1-15) and fowlicidin-2(19-31) caused only a negligible degree of cell death, when 100 μM was used with and without serum (FIG. 15C). Fowlicidin-2(1-18) and fowlicidin-2(15-31) caused 15% and 55% of cell death at 100 μM without serum, respectively, whereas serum reduced the cytotoxicity of both peptides to minimal 8%.

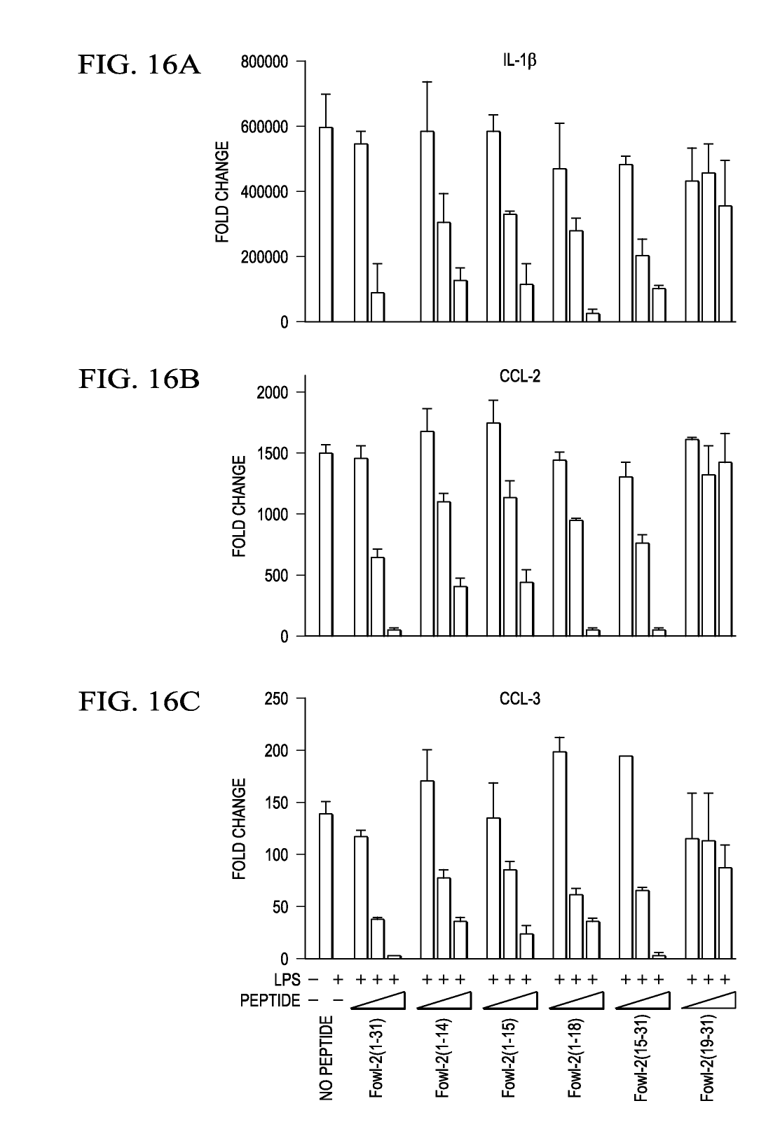

Because fowlicidin-2(1-14) and fowlicidin-2(19-31) displayed no toxicity at all against either human erythrocytes or epithelial cells, it is obvious that neither the N- or C-terminal α-helix alone is sufficient in the interaction with and lysis of mammalian cell membranes. Therefore, apparent cytoxicity of the full-length fowlicidin-2 must be caused by the amino acid sequence in the central kink region. However, the two analogs containing Arg¹⁵-Arg¹⁸ [fowlicidin-2(1-18) and fowlicidin-2(15-31)] exhibited a substantially reduced cytotoxicity, although such toxicity was higher than those of the peptide analogs containing the a-helix alone. The obvious reduction in toxicity associated with fowlicidin-2(1-18) and fowlicidin-2(15-31) is likely due to the conformational differences of the four-amino acid segment (Arg¹⁵-Arg¹⁸) in these two analogs as opposed in the full-length peptide.
LPS neutralization activities of fowlicidin-2 and its analogs—We have shown that fowlicidin-2 is capable of binding to LPS and further blocking LPS-induced inflammatory gene expression in RAW264.7 mouse macrophage cells (Example 1). To further map the region(s) responsible for the LPS-neutralizing activity, RAW264.7 cells were stimulated with 0.1 μg/ml of LPS for 4 in the presence or absence of three different concentrations of fowlicidin-2 and its analogs (1, 5, and 20 μM), and the expression levels of three common cytokine/chemokine genes, including IL-1β, CCL-2/MCP-1, and CCL-3//MIP-1a, were measured by real time RT-PCR as described (Example 1). The full-length peptide displayed potent LPS-neutralization, with >95% inhibition being observed with all three reference genes at 20 μM (FIG. 16). Two analogs containing the N-terminal α-helix [fowlicidin-2(1-14) and fowlicidin-2(1-15)] also suppressed LPS-induced inflammatory gene expressions in a dose-dependent manner, suggesting that the first 14 N-terminal residues, particularly the N-terminal α-helix, constitute a LPS binding and neutralization site. As expected, inclusion of cationic residues to the N-terminal α-helix [fowlicidin-2(1-18)] potentiated the binding and neutralization of anionic LPS, particularly at higher concentrations.

Interestingly, the analog containing entirely the C-terminal α-helix [fowlicidin-2(19-31)] showed no obvious inhibition of LPS-induced inflammatory gene expression even at 20 μM, indicating that, in contrary to the N-terminal α-helix, the C-terminal helix iteself is not sufficient in LPS binding and neutralization. The failure for fowlicidin-2(19-31) to interact with LPS is likely due to the fact that this segment is relatively hydrophobic with a net charge of +2, whereas the N-terminal 14 residues is amphipathic, with a net charge of +5. Inclusion of the cationic, four-amino acid segment to the C-terminal α-helix [fowlicidin-2(15-31)] showed potent, dose-dependent inhibition, implying the central kink region is critical in mediating the binding of cationic peptides to anionic LPS. Thus, the residues 15 to 31 are likely to constitute the second LPS binding and neutralization site, consistent with our earlier prediction with the full-length fowlicidin-2 based on the Limulus amebocyte lysate assay, in which at least two LPS-binding sites were predicted (Example 1). It is noted that neither peptide analogs alone caused alterations in the expression levels of three reference genes when used to stimulate RAW264.7 cells (data not shown), suggesting that the inhibitory effect was specifically due to LPS-peptide interactions.

### Discussion

A majority of α-helical antimicrobial peptides are composed of a predominant α-helix with a short central hinge sequence, due to the presence of a glycine (9,19). In contrast, fowlicidin-2 consists primarily of two short α-helices (residues 6-12 and 23-27), connected by an excessive kink region (residues 13-20) induced by Pro19 (not shown). The N-terminal α-helix adopts a typical amphipathic structure, whereas the C-terminal helix is more hydrophobic. The central kink region of fowlicidin-2 is highly positively charged containing six cationic residues. Fowlicidin-1, on the other hand, is composed of two hydrophobic α-helices separately by a slight kink caused by glycine (19). Most of the cationic residues of fowlicidin-1 are instead concentrated at both ends (19). Such structural differences between fowlicidin-2 and other α-helical antimicrobial peptides suggest that fowlicidin-2 might be functionally different, with distinct antimicrobial spectra or immunomodulatory activities.

By studying several fowlicidin-2 analogs with deletions of certain structural components, we have been able to determine the involvement of the two α-helices and central kink region in antibacterial, cytotoxicity, and LPS-neutralizing activities. We have revealed that the C-terminal a-helix alone [fowlicidin-2(19-31)] has no antibacterial, cytolytic or LPS-neutralizing activities. The N-terminal α-helix [fowlicidin-2(1-14)] is also weakly active against bacteria with relatively low capacity to bind and neutralize LPS and no toxicity to mammalian cells. These results indicated that the two α-helical segments per se are not sufficient for fowlicidin-2 to interact with LPS and prokaryotic and eukaryotic membranes and permeabilize membranes.

In contrast, addition of four amino acids in the central bending region (Arg¹⁵-Arg¹⁸) to the N- or C-terminal α-helical segment is always associated with a significant enhancement in both antibacterial and LPS-neutralizing activities, suggesting that this central region is critically important in membrane interactions. Given the highly positive charged feature of this segment due to the presence of three arginines, it is not surprising that this cationic region is capable of promoting the interactions of peptides with anionic LPS and bacterial membranes.

Cationicity, α-helicity and amphipathicity are among the most important physico-chemical parameters that dictate the functional properties of α-helical antimicrobial peptides (8-10). To a certain degree, these three parameters are often positively correlated with the antibacterial activity. Consistent with these observations, a gradual increase in cationicity, α-helicity and amphipathicity among all peptide analogs [fowlicidin-2(1-14), fowlicidin-2(1-15) and fowlicidin-2(1-18)] leads to a gradual enhancement in permeabilization of E. coli inner membranes (FIG. 14), LPS neutralization (FIG. 16) and antibacterial potency (FIG. 12).

A comparison of the functional properties of all peptide analogs with those of the full-length fowlicidin-2 has enabled identification of short analogs with better therapeutic potential. Although fowlicidin-2(1-14), fowlicidin-2(1-15), and fowlicidin-2(19-31) lack cytotoxicity, their antibacterial and LPS-neutralizing activities are disappointing, indicative of little clinical utility. On the other hand, relative of the parent peptide, fowlicidin-2(1-18) and fowlicidin-2(15-31) have a MIC of 2-4 μM against most bacteria tested, with a minimum reduction in bacterial killing activity (FIG. 12), but cause no hemolysis and minimum 8% death to human Caco-2 cells at 100 μM in the presence of serum (FIG. 15). Both fowlicidin-2(1-18) and fowlicidin-2(15-31) are further capable of blocking LPS-induced proinflammatory responses in RAW264.7 cells to a similar extent as the parent peptide (FIG. 16). Moreover, these two short peptide analogs kill bacteria irrespective of salt, and the antibacterial activity of fowlicidin-2(15-31) is not affected by serum (FIG. 12 and FIG. 13). Therefore, both peptides represent better candidates with a significant improvement in therapeutic window and safety as compared with the parent peptide. The in vivo toxicity and antibacterial and anti-LPS efficacy of fowlicidin-2(1-18) and fowlicidin-2(15-31) are currently under evaluation.

### References for Example 3

1. Hancock, R. E., and Diamond, G. (2000) Trends Microbiol. 8(9), 402-410
2. Zasloff, M. (2002) Nature 415(6870), 389-395
3. McPhee, J. B., and Hancock, R. E. (2005) J Pept Sci 11(11), 677-687
4. Oren, Z., and Shai, Y. (1998) Biopolymers 47(6), 451-463
5. Lee, I. H., Cho, Y., and Lehrer, R. I. (1997) Infect Immun 65(7), 2898-2903
6. Ciornei, C. D., Sigurdardottir, T., Schmidtchen, A., and Bodelsson, M. (2005) Antimicrob Agents Chemother 49(7), 2845-2850
7. Bartlett, K. H., McCray, P. B., Jr., and Thorne, P. S. (2004) Int J Antimicrob Agents 23(6), 606-612
8. Chen, Y., Mant, C. T., Farmer, S. W., Hancock, R. E., Vasil, M. L., and Hodges, R. S. (2005) J Biol Chem 280(13), 12316-12329
9. Tossi, A., Sandri, L., and Giangaspero, A. (2000) Biopolymers 55(1), 4-30
10. Dathe, M., and Wieprecht, T. (1999) Biochim Biophys Acta 1462(1-2), 71-87
11. Zanetti, M., Gennaro, R., Skerlavaj, B., Tomasinsig, L., and Circo, R. (2002) Curr Pharm Des 8(9), 779-793
12. Bals, R., and Wilson, J. M. (2003) Cell Mol Life Sci 60(4), 711-720
13. Zaiou, M., and Gallo, R. L. (2002) J Mol Med 80(9), 549-561
14. Lehrer, R. I., and Ganz, T. (2002) Curr Opin Hematol 9(1), 18-22
15. Cirioni, O., Giacometti, A., Ghiselli, R., Bergnach, C., Orlando, F., Silvestri, C., Mocchegiani, F., Licci, A., Skerlavaj, B., Rocchi, M., Saba, V., Zanetti, M., and Scalise, G. (2006) Antimicrob Agents Chemother 50(5), 1672-1679
16. Giacometti, A., Cirioni, O., Ghiselli, R., Bergnach, C., Orlando, F., D'Amato, G., Mocchegiani, F., Silvestri, C., Del Prete, M. S., Skerlavaj, B., Saba, V., Zanetti, M., and Scalise, G. (2004) Crit Care Med 32(12), 2485-2490
17. Giacometti, A., Cirioni, O., Ghiselli, R., Mocchegiani, F., D'Amato, G., Circo, R., Orlando, F., Skerlavaj, B., Silvestri, C., Saba, V., Zanetti, M., and Scalise, G. (2004) Am J Respir Crit Care Med 169(2), 187-194
18. Example 1
19. Xiao, Y., Dai, H., Bommineni, Y. R., Soulages, J. L., Gong, Y. X., Prakash, O., and Zhang, G. (2006) FEBS J 273(12), 2581-2593
20. Sreerama, N., Venyaminov, S. Y., and Woody, R. W. (2000) Anal Biochem 287(2), 243-251
21. Wüthrich, K. (1986) NMR of proteins and nucleic acids, Wiley, New York
22. Wüthrich, K., Billeter, M., and Braun, W. (1983) J Mol Biol 169(4), 949-961
23. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998) Acta Crystallogr D Biol Crystallogr 54 (Pt 5), 905-921
24. Nicholls, A., Sharp, K. A., and Honig, B. (1991) Proteins 11(4), 281-296
25. Dorschner, R. A., Lopez-Garcia, B., Peschel, A., Kraus, D., Morikawa, K., Nizet, V., and Gallo, R. L. (2006) Faseb J 20(1), 35-42
26. Steinberg, D. A., and Lehrer, R. I. (1997) Methods Mol Biol 78, 169-186
27. Turner, J., Cho, Y., Dinh, N. N., Waring, A. J., and Lehrer, R. I. (1998) Antimicrob Agents Chemother 42(9), 2206-2214
28. Laskowski, R. A., Rullmannn, J. A., MacArthur, M. W., Kaptein, R., and Thornton, J. M. (1996) J Biomol NMR 8(4), 477-486
29. Yu, K., Park, K., Kang, S. W., Shin, S. Y., Hahm, K. S., and Kim, Y. (2002) J Pept Res 60(1), 1-9
30. Tack, B. F., Sawai, M. V., Kearney, W. R., Robertson, A. D., Sherman, M. A., Wang, W., Hong, T., Boo, L. M., Wu, H., Waring, A. J., and Lehrer, R. I. (2002) Eur J Biochem 269(4), 1181-1189
31. Travis, S. M., Anderson, N. N., Forsyth, W. R., Espiritu, C., Conway, B. D., Greenberg, E. P., McCray, P. B., Jr., Lehrer, R. I., Welsh, M. J., and Tack, B. F. (2000) Infect Immun 68(5), 2748-2755

### Example 4

### Fowlicidin-3 is an α-Helical Cationic Host Defense Peptide with Potent Antibacterial and Lipopolysaccharide-Neutralizing Activities

Cationic antimicrobial peptides comprise a large group of small peptides with extremely diverse amino acid sequences but with conserved features in each family [1, 2]. Acting as an important first line of defense, these peptides are mostly produced by innate immune cells such as phagocytes, mucosal epithelial cells, and skin keratinocytes in vertebrates, capable of killing a broad range of bacteria, fungi, and viruses, including resistant strains [1, 2]. Because of non-specific membrane-lytic activities, antimicrobial peptides have a low tendency to develop resistance, a desirable feature as a new class of antimicrobial agents [1, 3, 4].

Besides having direct microbicidal activities, antimicrobial peptides have increasingly been appreciated to play a profound role in regulating host immune responses to infections. Many peptides have been shown to be actively involved in binding and neutralization of LPS, chemotaxis of immune cells, regulation of dendritic cell differentiation, induction of angiogenesis and re-epithelialization, and modulation of cytokine and chemokine gene expression [5-7]. To better reflect the pleiotropic effects of antimicrobial peptides on various aspects of innate and adaptive immunity, these peptides have been proposed to be renamed as host defense peptides [6, 7]. Both antimicrobial and immunomodulatory activities of these peptides are being harnessed and manipulated for therapeutic benefit. It is possible to employ these peptides for antimicrobial therapy without provoking detrimental proinflammatory responses [6-8].

Cathelicidins represent a major family of host defense peptides that have been identified in fish, birds, and mammals [9-11]. All cathelicidins share a highly conserved “cathelin” pro-sequence at the N-terminus, with extremely variable C-terminal sequences having antimicrobial and immune regulatory activities [9-11]. We recently identified three chicken cathelicidins, namely fowlicidins 1-3, and found that putatively mature fowlicidin-1 and -2 are among the most efficacious cathelicidins that have been reported, with fowlicidin-1 being slightly more potent than fowlicidin-2 in killing bacteria [12; Example 2].

Here we report structural and functional characterization of fowlicidin-3, a third chicken cathelicidin that is likely to have evolved from fowlicidin-1 by gene duplication [Example 2]. Similar to fowlicidin-1, putatively mature fowlicidin-3 peptide was found to be largely α-helical with a kink in the central region and a relatively flexible unstructured segment in the N-terminal region. Fowlicidin-3 is highly active against a broad range of bacteria in vitro, including antibiotic-resistant strains, but 4- to 6-fold less toxic to mammalian host cells than fowlicidin-1. Moreover, fowlicidin-3 is more potent than fowlicidin-1 in blocking LPS-induced proinflammatory responses. Therefore, fowlicidin-3 should be an attractive antibacterial and anti-sepsis drug.

### Materials and Methods

### Peptide Synthesis

Putatively mature forms of fowlicidin-1 and -3 were predicted to consist of 26 and 27 amino acid residues in the C-terminal regions of their precursors, respectively [Example 1]. Fowlicidin-1 (RVKRVWPLVIRTVIAGYNLYRAIKKK, SEQ ID NO: 2) and -3 (KRFWPLVPVAINTVAAGIN LYKAIRRK, SEQ ID NO: 18) were chemically synthesized using the standard solid-phase method by Bio-Synthesis (Lewisville, Tex.). Both peptides were purified to >95% purity by reverse phase high-performance liquid chromatography (RP-HPLC). The mass and purity of each peptide were further confirmed by matrix-assisted laser desorption/ionization time-of-flight (MALDI-TOF) mass spectrometry using the Voyager DE-PRO instrument (Applied Biosystems, Foster City, Calif.) housed in the Recombinant DNA/Protein Core Facility of Oklahoma State University. Molecular masses of the two peptides are as follows: fowlicidin-1 (calculated: 3141.9 and observed: 3141.6) and fowlicidin-3 (calculated: 3094.8 and observed: 3095.1). Lyophilized peptides were reconstituted in 0.01% acetic acid. The peptide concentrations were measured by UV absorbance at 280 nm in the presence of 6 M guanidine hydrochloride [25], based on the extinction coefficients for aromatic tryptophan and tyrosine residues present in both peptides.

### CD Spectroscopy and Secondary Structure Determination

The secondary structure of fowlicidin-3 was determined on a Jasco-715 spectropolarimeter using a 0.1-cm path length cell over the 180-260 nm range as described [Example 2].

### NMR Spectroscopy and Tertiary Structure Calculations

The NMR experiments were performed with 500-MHz Varian UNITY plus NMR spectrometer (Varian, Palo Alto, Calif.) as previously described [Example 2].

### Bacterial Culture and Antibacterial Testing

Gram-negative bacteria (Escherichia coli ATCC 25922, S. enterica serovar Typhimurium ATCC 14028, S. enterica serovar Typhimurium DT104 ATCC 700408, and Klebsiella pneumoniae ATCC 13883), and Gram-positive bacteria (Listeria monocytogenes ATCC 19115, S. aureus ATCC 25923, S. aureus ATCC BAA-39, and S. aureus ATCC 43300) were purchased from either ATCC (Manassas, Va.) or MicroBiologics (St. Cloud, Minn.) and tested individually against fowlicidin-1 and -3. The MICs were determined by a standard broth microdilution assay as recommended by the Clinical and Laboratory Standards Institute [15]. Briefly, overnight bacterial culture was subcultured in fresh trypticase soy broth (TSB) with shaking at 250 rpm at 37° C. for 3 h to reach the mid-log phase of growth. Bacteria were then washed twice in 10 mM sodium phosphate buffer, pH 7.4, and diluted to 5×105 CFU/ml in MHB (BBL, Cockeysville, Md.). Following dispensing 90 μl of bacteria into 96-well cell culture plates, 10 μl of peptides in serial two-fold dilutions were added in duplicate. The MIC value of each peptide was determined as the lowest peptide concentration that gave no visible bacterial growth after overnight incubation at 37° C.

### Cytoplasmic Membrane Permeabilization Assay

E. coli ML-35p, a lactose permease-deficient strain with constitutive production of β-galactosidase in the cytosol, was used as described [17-19]. Briefly, mid log-phase bacteria were washed twice in 10 mM sodium phosphate buffer, pH 7.4, diluted to 0.03 OD600 (equivalent to 2.5-5×10⁷ CFU/ml) in the same phosphate buffer containing 1% TSB with and without 100 mM of NaCl. Following dispensing 80 μl bacteria into each well of a 96-well tissue culture plate, different concentrations of fowlicidins and 1.5 mM of p-nitrophenyl-β-D-galactopyranoside (ONPG) (Sigma, St. Louis, Mo.), a chromogenic substrate for β-galactosidase, were added to a total volume of 100 μl per well. The production of p-nitrophenol was monitored spectrophotometrically at 37° C. and 405 nm every 2 min for up to 1 h with periodic shaking. It is noted that, the amount of cytosolic β-galactosidase released, as indicated by the amount of p-nitrophenol produced, is proportional to the degree of lysis of E. coli inner membranes [17-19].

### Serum Effect on the Antibacterial Activity of Fowlicidin-3

Radial diffusion assay [22] was used to study the effect of serum on the antibacterial activity of fowlicidins. Briefly, after solidification of the underlay gel containing 4×10⁵ CFU/ml of a reference strain of S. aureus ATCC 25923, small wells (˜3 mm in diameter) were punched. One μg of fowlicidin-1 or -3 was diluted to a total of volume of 4 μl in 0.01% acetic acid with or without 50% chicken or human serum and then added separately to the wells. After 3 h of diffusion at 37° C., the nutrient rich overlay gel was poured and incubated at 37° C. overnight. The diameters of bacterial clearance zone were measured.

### Cytotoxicity Assay

The toxicity of fowlicidin-3 toward mammalian epithelial cells was evaluated by using MDCK cells (ATCC) and an alamarBlue dye (Biosource, Camarillo, Calif.) as described [Example 1].

### Hemolysis Assay

Freshly collected chicken and human blood were used for evaluating hemolytic activity as described [Examples 1 and 2].

### Real-Time PCR Analysis of the Effect of Fowlicidins on LPS-Induced Proinflammatory Gene Expression

Mouse macrophage RAW 264.7 cells were used to study the modulation of LPS-induced cytokine/chemokine gene expression by fowlicidin-3 in comparison with fowlicidin-1. Cells were seeded in 12-well tissue culture plates with 5×10⁵ cells/well in DMEM containing 10% FBS. After overnight incubation, cells were pretreated for 30 min with 0.5, 2.5, and 10 μM of fowlicidins in duplicate, followed by stimulation for 4 h with 100 ng/ml LPS from E. coli 0114:B4 (Sigma). Total RNA was then isolated from cells using TRIzol (Invitrogen, Carlsbad, Calif.) according to the manufacturer's instructions. Quantitative real-time reverse transcriptase (RT)-PCR was used to analyze the expressions of three common proinflammatory genes, namely IL-1β, CCL2/MCP-1, and CCL3/MIP-1a, using exon-spanning primers as described [Example 1].

The first-strand cDNA from 1.5 μg of each RNA sample was synthesized in a reaction volume of 20 μl at 42° C. for 30 min by using QuantiTect® Reverse Transcription Kit (Qiagen, Valencia, Calif.), which included removal of genomic DNA contamination prior to cDNA synthesis. Real-time PCR was performed by using 0.2 μg of the first-strand cDNA, gene-specific primers, SYBR® Premix Ex Taq™ (Takara Bio, Japan), and MyiQ® Real-Time PCR Detection System (Bio-Rad, Hercules, Calif.) in a total volume of 10 μ. PCR cycling conditions were as follows: 95° C. for 30 sec, followed by 40 cycles of 95° C. for 15 sec, 55° C. for 30 sec, and 72° C. for 30 sec. The comparative ΔΔCT method was used to quantify the gene expression levels, where β-actin was used as an internal control for normalization [12]. Relative fold changes in gene expression were calculated using the formula 2ΔΔCt. Melting curve analysis (55-95° C.) was performed and confirmed amplification of a single product in each case.

### Results

### Structural Characterization of Fowlicidin-3

Circular dichroism (CD) spectroscopy was first performed to determine the secondary structure of fowlicidin-3 in the presence of different concentrations of trifluoroethanol (TFE) and SDS. Fowlicidin-3 was largely unstructured in phosphate buffer and began to transform into a typical α-helical conformation following addition of increasing concentrations of TFE. Significant α-helical content (86%) with virtually no β-sheet structure was observed with fowlicidin-3 in 60% TFE (not shown). Similarly, fowlicidin-3 also exhibited 53% α-helical content in the presence of 0.25% SDS micelles (data not shown). These results suggested that fowlicidin-3 is likely to adopt a predominant α-helical conformation when interacting with bacterial membranes.

To further determine the tertiary structure of fowlicidin-3, two-dimensional nuclear magnetic resonance (NMR) spectroscopy was employed with the peptide in the presence of 50% deuterated TFE (TFE-d3)/50% H₂O (v/v) as described [13; Example 2]. Complete proton resonance assignments were obtained using spin system identification and sequential assignments from NMR spectra recorded at 25° C. Consistent with the CD results, the Ca-proton chemical shift index, together with the presence of a number of sequential dNN(i, i+1), nonsequential d_{αN}(i, i+3), and d_{αβ}(i, i+3) NOE peaks, was clearly indicative of an α-helical conformation for fowlicidin-3.

A total of 205 NOE constraints, including 68 intraresidue, 86 sequential, and 51 medium range constraints, were used to calculate the tertiary structure of fowlicidin-3. From 100 calculated structures that satisfied the experimental restraints, 20 structures with the lowest total energy were selected for further analysis. A Ramachandran plot, produced by PROCHECK-NMR [14], showed that 64.8% residues are in the most favored region and 33.4% are in additional allowed regions, respectively. A superimposition of 20 lowest energy structures showed a considerable degree of flexibility with a pairwise root mean square deviation (rmsd) of backbone of 3.03 Å. However, alignments along residues V⁹-A¹⁶ and N¹⁹-R²⁵ of the 20 structures resulted in the backbone rmsd values of <0.4 Å in both cases, suggesting relative rigidity of these two α-helical segments.

The energy minimized average structure of fowlicidin-3 was further calculated, showing a predominant α-helical structure extending from V⁹-R²⁵ with a relatively flexible N-terminal segment (FIG. 17A). A closer examination of the NMR structure revealed a kink within the long a-helix between residues 16-19, due to the presence of a glycine residue at position 17. As evidenced by a lack of NOEs, such a kink provides conformational flexibility between two short α-helical segments, reminiscent of fowlicidin-1 [Example 2]. Superimposition of fowlicidin-1 with fowlicidin-3 indeed revealed substantial overlapping, except for the flexible N-sequences. This is perhaps not surprising, given the fact that both peptides are likely to have evolved by duplication and share >60% identity in amino acid sequence in the putatively mature region (FIG. 17B).

### Evaluation of Antibacterial Properties of Fowlicidin-3

Fowlicidin-1 was found to be among the most potent cathelicidins in killing bacteria [Example 1]. To evaluate the antibacterial spectrum and efficacy of fowlicidin-3, we performed standard broth microdilution assays in 100% Muller-Hinton broth (MHB) as recommended by the Clinical and Laboratory Standards Institute [15] using fowlicidin-1 as a reference peptide. As shown in Table VII, fowlicidin-3 was active against a wide range of Gram-negative and Gram-positive bacteria with the minimum inhibitory concentrations (MICs) in the range of 1-2 μM, frequently showing slightly higher potency than fowlicidin-1. Moreover, fowlicidin-3 exhibited no diminished efficiency against antibiotic-resistant strains, including multidrug-resistant Salmonella enterica serovar Typhimurium DT104 and two methicillin-resistant Staphylococcus aureus (MRSA) strains tested.

TABLE VII
MICs of fowlicidin-3 in comparison with fowlicidin-1^{a}

| Bacteria | ATCC No. | Fowl-3 (μM) | Fowl-1(μM) |
|---|---|---|---|
| Gram-negative: | | | |
| E. coli | 25922 | 2 | 2 |
| S. typhimurium | 14028 | 2 | 2 |
| S. enteritidis | 13076 | 2 | 2 |
| K. pneumoniae | 13883 | 1 | 1 |
| S. typhimurium DT104 | 700408 | 2 | 2 |
| Gram-positive: | | | |
| L. monocytogenes | 19115 | 2 | 2 |
| S. aureus | 25923 | 1 | 2 |
| S. aureus (MRSA) | 43300 | 1 | 2 |
| S. aureus (MRSA) | BAA-39 | 1 | 1 |

^{a}MICs were determined as the lowest peptide concentration that gave no visible bacterial growth after overnight incubation in a standard CLSI broth microdilution assay using 100% Muller-Hinton broth. The experiments were repeated at least twice for each bacterial strain with similar values.

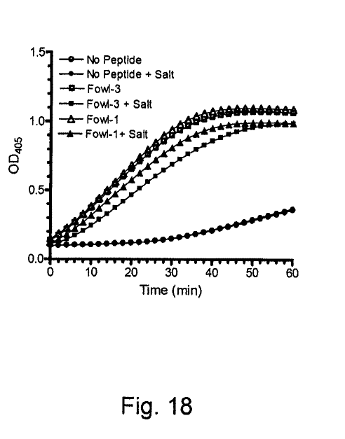

Most cationic host defense peptides including cathelicidins are membrane-lytic agents, killing bacteria by physical interaction with and disruption of bacterial cell membranes, although increasing evidence suggests the presence of intracellular targets for certain peptides [1, 16]. To examine the mechanism of action and bacterial killing kinetics of fowlicidin-3, E. coli ML-35p, a strain that contains a plasmid giving constitutive expression of β-galactosidase in the cytosol, was incubated with different concentrations of peptides for 1 h in the presence of a chromogenic substrate, p-nitrophenyl-β-D-galactopyranoside (ONPG) [17-19]. It is conceivable that the amount of β-galactosidase released, as indicated by color change, is proportional to the degree of permeabilization of bacterial cytosolic membranes by fowlicidins. As shown in FIG. 18, membrane lysis began almost immediately upon addition of 1 μM fowlicidin-3 or -1 to bacteria, reaching a plateau around 30-40 min, entirely consistent with earlier colony counting assays with fowlicidin-1 in which bacteria were killed quickly with the maximum killing occurring at 30 min following incubation of the peptide with bacteria [Example 1]. Identical trends were also observed with 0.5 and 2 μM of fowlicidins (data not shown). These results implied that, as most other host defense peptides, physical membrane disruption appears to be a major mechanism of killing bacteria for fowlicidin-3 and -1.

Physiological concentrations of salt prove to be inhibitory to antibacterial activities of many antimicrobial peptides, such as human cathelicidin LL-37 [18] and α- and β-defensins [20, 21]. However, the presence of 100 mM NaCl had little impact on membrane lysis with only a minimal delay in killing kinetics for fowlicidin-3 (FIG. 18), consistent with our direct colony counting assay (data not shown). These data suggested that, similar to fowlicidin-1 and -2 [Example 1], fowlicidin-3 kills bacteria in a salt-independent manner, in contrast with many other peptides whose activities are severely suppressed in the presence of salt [18, 20, 21].

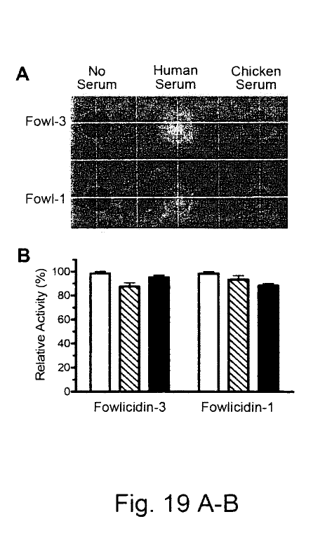

Serum has been found to be another important inhibitory factor in bactericidal activities of many host defense peptides, probably due to the presence of certain salts, divalent cations, and peptide-binding proteins. To examine the effect of serum on antibacterial efficacy of fowlicidin-3, a radial diffusion assay [22] was performed with S. aureus ATCC 25923 and peptides diluted with and without 50% human or chicken serum. The results revealed that both fowlicidin-3 and -1 retained >80% antibacterial activity in either serum (FIG. 19A and B), implying their in vivo therapeutic potential for systemic applications.

### Evaluation of the Toxicity of Fowlicidin-3 to Mammalian Cells

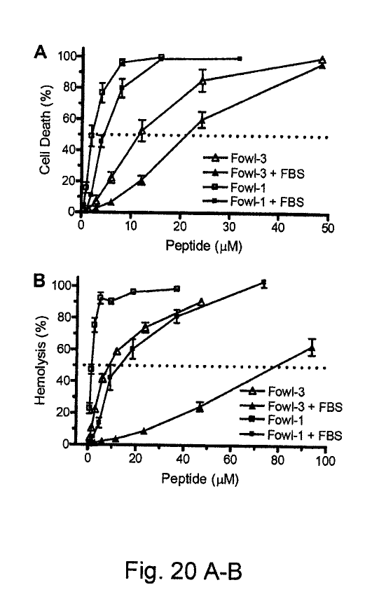

As compared with β-sheet defensins, a considerably higher degree of toxicity to mammalian cells occurs with α-helical cathelicidins, limiting their potential as antimicrobial agents. To study the toxicity of fowlicidin-3, Madin-Darby canine kidney (MDCK) epithelial cells were first incubated with different concentrations of fowlicidins in the presence or absence of 10% FBS, followed by a cell viability assay as described [Example 1]. As compared with fowlicidin-1 with a 50% effective concentration (EC₅₀) of ˜2 μM, fowlicidin-3 killed 50% MDCK cells at 12 μM (FIG. 6A). Moreover, the presence of 10% serum further reduced the toxicity of fowlicidin-3 by 2-fold (FIG. 20A).

To further test the hemolytic activity of fowlicidin-3, freshly isolated human erythrocytes were incubated with fowlicidins with and without 10% FBS, and lysis of erythrocytes was measured according to the release of hemoglobin [Example 1]. In the absence of serum, 50% hemolysis occurred at 9μM for fowlicidin-3, whereas fowlicidin-1 was considerably toxic with an EC₅₀ of ˜1.5 μM (FIG. 20B). Serum substantially reduced hemolysis of both peptides, with EC₅₀ values of 80 μM for fowlicidin-3 and 13 μM for fowlicidin-1 in 10% FBS. Taken together, fowlicidin-3 is slightly more potent than fowlicidin-1 in killing many bacterial strains tested, but appears to be 4- to 6-fold less toxic to mammalian cells than fowlicidin-1.

### Inhibition of LPS-Induced Proinflammatory Gene Expression by Fowlicidin-3

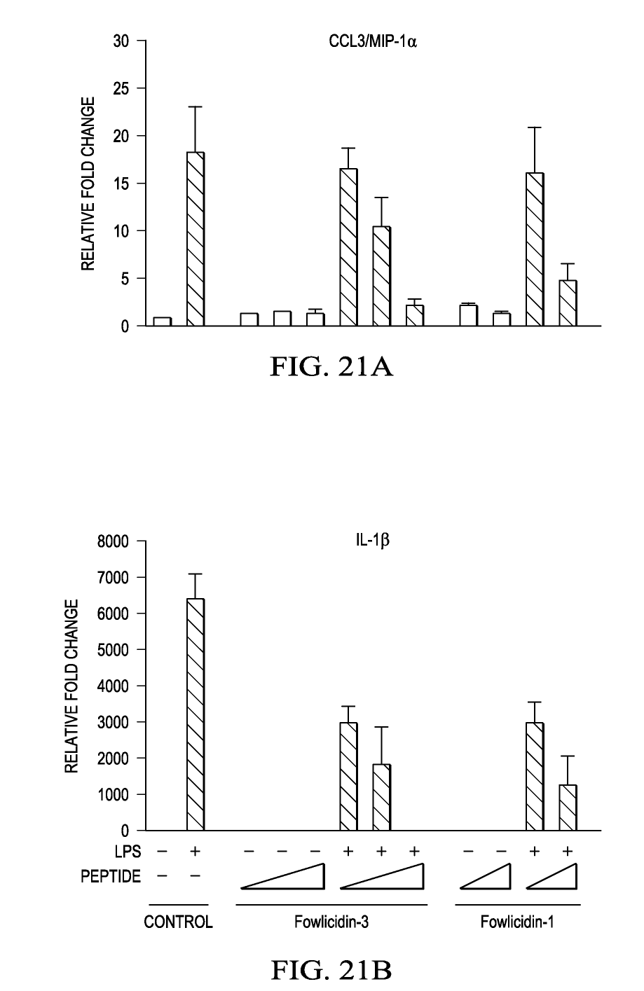

Because fowlicidin-1 and -2 were found to be able to bind LPS directly and suppressed LPS-induced cytokine gene expression [Example 1], we sought to determine whether fowlicidin-3 has a similar LPS-neutralizing activity. Mouse macrophage RAW264.7 cells were stimulated for 4 h with 100 ng/ml LPS in the presence and absence of different concentrations of fowlicidins, followed by real-time RT-PCR analysis of the expressions of three common proinflammatory genes, including interleukin (IL)-1β, CC chemokine ligand 2 (CCL2)/MCP-1, and CCL3/MIP-1a. As shown in FIGS. 21A and B, fowlicidin-3 dose-dependently inhibited the expression of IL-1β or CCL3/MIP-1a genes, with 10 μM reducing expression of both genes by >95%. A similar blockage of CCL2/MCP-1 expression was also observed (data not shown). Because treating cells with fowlicidin-3 alone had no effect on gene expression (FIG. 21), such an LPS-neutralizing activity was specific. It is noteworthy that, as compared with fowlicidin-1, fowlicidin-3 is more potent in inhibiting LPS-induced gene expression (FIG. 21), suggesting that fowlicidin-3 may be more effective in anti-sepsis therapy.

### Discussion

Our previous analyses of genomic sequences have revealed that the genes for fowlicidin-1 and -3 are almost identical in the first three exons and first three introns [Example 1]. The fourth exon, which primarily encodes biologically active, mature sequences, also shares >60% identity between the two peptides (FIG. 17B). Therefore, these two fowlicidins are most likely to be duplicated from each other during evolution. The putatively mature fowlicidin-3 peptide consists of 27 amino acid residues with a charge of +6 and no anionic residues, whereas fowlicidin-1 is composed of 26 amino acids with a net charge of +8. Evolution of two highly similar antimicrobial peptides with potent antibacterial activities could represent an enforcement of innate host defense. It is also plausible that fowlicidin-1 and -3 could have some non-overlapping biological functions yet to be discovered.

Because of a similarity in primary sequence, it is not surprising that both fowlicidins adopt a similar α-helical conformation in membrane-mimicking environments (FIG. 17B). Moreover, both peptides contain a kink near the central helical region due to the presence of a conserved glycine residue (FIG. 17B). Interestingly, such a glycine-induced hinge is not unique to fowlicidins, but appears to be a common feature for many α-helical cationic host defense peptides [13, 23, 24]. The presence of a hinge structure has been shown to be beneficial in enhancing molecular flexibility while reducing the toxicity of otherwise rigid peptides to mammalian cells [23, 24].

Amphipathicity is another hallmark of most α-helical cationic peptides [23, 24]. However, unlike typical α-helical peptides, the long helices of fowlicidin-1 and -3 are much less amphipathic, with no obvious segregation of hydrophobic residues from hydrophilic residues. Furthermore, the α-helical region is highly hydrophobic in that fowlicidin-3 is composed of only one cationic (K²²) and two polar uncharged residues (N¹², T¹³ and N¹⁹), whereas fowlicidin-1 consists of only two cationic (R¹¹ and R²¹) and two polar uncharged residues (T¹² and N¹⁸) (FIG. 17B). Instead, positively charged residues are mostly concentrated at both tails (FIG. 17B).

A series of antibacterial tests revealed that, similar to fowlicidin-1, fowlicidin-3 possesses potent, broad-spectrum, and fast-acting bactericidal activities with similar efficiency against both antibiotic-susceptible and -resistant bacterial strains. Killing of bacteria by fowlicidins starts immediately upon contact with bacteria, in sharp contrast with human cathelicidin LL-37, which takes up to 20-30 min before lysis of bacterial inner membranes occurs [17, 18]. More significantly, bacterial killing activity is largely unaffected by salt or serum, making fowlicidins attractive therapeutic candidates for potential in vivo systemic applications.

In spite of similarities in structural and antibacterial properties, fowlicidin-3 is much less toxic to mammalian cells than fowlicidin-1. Because the cytotoxicity (EC₅₀) of fowlicidin-3 is at least 10-to 40-fold (in the presence of serum) higher than MICs against all bacterial strains tested, a therapeutic window clearly exists for fowlicidin-3 particularly for systemic applications. More desirably, fowlicidin-3 is highly potent in blocking LPS-induced proinflammatory gene expression. Collectively, fowlicidin-3 appears to have promising therapeutic potential for further development as a novel antimicrobial and antisepsis agent.

It is interesting to note that the relatively higher toxicity associated with fowlicidin-1 is likely due to limited flexibility of the a-helix, which is a result of the physical hindrance caused by the side chain of a nearby tyrosine [Example 2]. Although fowlicidin-3 is devoid of aromatic residues adjacent to the conserved glycine (FIG. 17B), it will be important to examine the impact of further enhancing its flexibility on the functional properties. In fact, the flexibility of the hinge region has often been found to be positively correlated with an decrease in the toxicity of many α-helical peptides [23, 24]. Because amphipathicity, hydrophobicity, and helicity are among the most important factors that influence the antibacterial and toxicity of α-helical cationic peptides [23, 24], rational changes of these structural and physico-chemical parameters are likely to further improve the therapeutic potential of fowlicidin-3.

### References for Example 4

1. Zasloff, M. (2002) Antimicrobial peptides of multicellular organisms. Nature 415, 389-395.
2. Brogden, K. A., Ackermann, M., McCray, P. B., Jr. & Tack, B. F. (2003) Antimicrobial peptides in animals and their role in host defences. Int J Antimicrob Agents 22, 465-78.
3. Hancock, R. E. & Patrzykat, A. (2002) Clinical development of cationic antimicrobial peptides: from natural to novel antibiotics. Curr Drug Targets Infect Disord 2, 79-83.
4. Zanetti, M., Gennaro, R., Skerlavaj, B., Tomasinsig, L. & Circo, R. (2002) Cathelicidin peptides as candidates for a novel class of antimicrobials. Curr Pharm Des 8, 779-93.
5. Yang, D., Biragyn, A., Hoover, D. M., Lubkowski, J. & Oppenheim, J. J. (2004) Multiple roles of antimicrobial defensins, cathelicidins, and eosinophil-derived neurotoxin in host defense. Annu Rev Immunol 22, 181-215.
6. McPhee, J. B. & Hancock, R. E. (2005) Function and therapeutic potential of host defence peptides. J Pept Sci 11, 677-87.
7. Bowdish, D. M., Davidson, D. J. & Hancock, R. E. (2005) A re-evaluation of the role of host defence peptides in mammalian immunity, Curr Protein Pept Sci 6, 35-51.
8. Finlay, B. B. & Hancock, R. E. (2004) Can innate immunity be enhanced to treat microbial infections? Nat Rev Microbiol 2, 497-504.
9. Zanetti, M. (2004) Cathelicidins, multifunctional peptides of the innate immunity. J Leukoc Biol 75, 39-48.
10. Zaiou, M. & Gallo, R. L. (2002) Cathelicidins, essential gene-encoded mammalian antibiotics. J Mol Med 80, 549-61.
11. Lehrer, R. I. & Ganz, T. (2002) Cathelicidins: a family of endogenous antimicrobial peptides. Curr Opin Hematol 9, 18-22.
12. Example 1.
13. Example 2
14. Laskowski, R. A., Rullmannn, J. A., MacArthur, M. W., Kaptein, R. & Thornton, J. M. (1996) AQUA and PROCHECK-NMR: programs for checking the quality of protein structures solved by NMR. J Biomol NMR 8, 477-86.
15. National Committee for Clinical Laboratory Standards. (2003) Methods for dilution antimicrobial susceptibility tests for bacteria that grow aerobically, 6th ed. Approved Standard M7-A6, Wayne, Pa.
16. Brogden, K. A. (2005) Antimicrobial peptides: pore formers or metabolic inhibitors in bacteria? Nat Rev Microbiol 3, 238-50.
17. Dorschner, R. A., Lopez-Garcia, B., Peschel, A., Kraus, D., Morikawa, K., Nizet, V. & Gallo, R. L. (2006) The mammalian ionic environment dictates microbial susceptibility to antimicrobial defense peptides. FASEB J 20, 35-42.
18. Turner, J., Cho, Y., Dinh, N. N., Waring, A. J. & Lehrer, R. I. (1998) Activities of LL-37, a cathelin-associated antimicrobial peptide of human neutrophils. Antimicrob Agents Chemother 42, 2206-2214.
19. Friedrich, C., Scott, M. G., Karunaratne, N., Yan, H. & Hancock, R. E. (1999) Salt-resistant alpha-helical cationic antimicrobial peptides. Antimicrob Agents Chemother 43, 1542-8.
20. Hoover, D. M., Wu, Z., Tucker, K., Lu, W. & Lubkowski, J. (2003) Antimicrobial characterization of human beta-defensin 3 derivatives. Antimicrob Agents Chemother 47, 2804-9.
21. Ericksen, B., Wu, Z., Lu, W. & Lehrer, R. I. (2005) Antibacterial activity and specificity of the six human alpha-defensins. Antimicrob Agents Chemother 49, 269-75.
22. Steinberg, D. A. & Lehrer, R. I. (1997) Designer assays for antimicrobial peptides. Disputing the “one-size-fits-all” theory. Methods Mol Biol 78, 169-86.
23. Tossi, A., Sandri, L. & Giangaspero, A. (2000) Amphipathic, alpha-helical antimicrobial peptides. Biopolymers 55, 4-30.
24. Dathe, M. & Wieprecht, T. (1999) Structural features of helical antimicrobial peptides: their potential to modulate activity on model membranes and biological cells. Biochim Biophys Acta 1462, 71-87.
25. Gill, S. C. & von Hippel, P. H. (1989) Calculation of protein extinction coefficients from amino acid sequence data. Anal Biochem 182, 319-26.
26. Sreerama, N., Venyaminov, S. Y. & Woody, R. W. (2000) Estimation of protein secondary structure from circular dichroism spectra: inclusion of denatured proteins with native proteins in the analysis. Anal Biochem 287, 243-51.
27. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. & Warren, G. L. (1998) Crystallography & NMR system: A new software suite for macromolecular structure determination. Acta Crystallogr D Biol Crystallogr 54, 905-21.
28. Koradi, R., Billeter, M. & Wuthrich, K. (1996) MOLMOL: a program for display and analysis of macromolecular structures. J Mol Graph 14, 51-55.

### Example 5

### Tryptophan is Critical in Maintaining the Antibacterial Activity of Fowlicidin-1

### Abstract

Fowlicidin-1 has been found to possess potent antibacterial activity against a broad range of Gram-negative and Gram-positive bacteria, including antibiotic-resistant strains. To further determine the involvement of the first eight N-terminal amino acids in the antibacterial activity of fowlicidin-1, four peptide analogs with sequential deletions of amino acids from positions 5 to 7 were synthesized. The standard broth microdilution assay was used to test the activities of these four peptides against three Gram-positive and two Gram-negative bacteria, including multidrug-resistant strains S. aureus ATCC 43300 and S. enterica serovar Typhimurium DT104 ATCC 700408. Interestingly, fowlicidin-1 (5-26) and fowlicidin-1(6-26) showed identical MICs against all five bacterial strains tested, clearly suggesting that valine at position 5 is dispensable for the antibacterial activity. Fowlicidin-1(7-26) with omission of typtophan at position 6 resulted in a sharp=4-fold reduction in bacteria-killing efficiency, indicating the critical involvement of tryptophan in the antibacterial activity of fowlicidin-1. Fowlicidin-1(8-26) with an additional deletion of proline 7 displayed low antibacterial activities similar to fowlicidin-1(7-26), implying that proline 7 is not required for maintaining the antibacterial activity. Taken together, fowlicidin-1(6-26), retaining potent antibacterial activity with a significantly reduced cytotoxicity, is a short peptide analog with enhanced therapeutic potential as an antimicrobial drug candidate.

### Introduction

Cationic antimicrobial peptides are naturally occurring antibiotics that are actively being explored as a new class of anti-infective agents. We recently identified three cathelicidin antimicrobial peptides from chicken, namely fowlicidins 1-3, with potent and broad-spectrum antibacterial activities in vitro (see Example 1). They can kill antibiotic-resistant bacteria equally as susceptible bacteria. Furthermore, we have shown that three fowlicidins possess strong lipopolysaccharide (LPS)-neutralizing activity, suppressing LPS-induced proinflammatory cytokine gene expression to a minimum level. Collectively, these fowlicidin peptides represent excellent candidates for further development as a new generation of antimicrobial drugs in light of rapid emergence of antibiotic resistance worldwide. We recently have solved the NMR structures of these three fowlicidins, providing a timely opportunity for structure-based mutagenesis with a goal of potentiating their antibacterial activities while reducing their unwanted side-effects. This Example systematically explores structure-activity relationship studies of fowlicidin-1 to develop novel therapeutics with desirable functions.

### Materials and Methods

Design and synthesis of fowlicidin-1 analogs. We have revealed that fowlicidin-1 consists of a highly flexible, unstructured segment at the N-terminus from amino acid residues 1-8 and that the first five residues play a minimum role in antibacterial, LPS-binding, and cytotoxic activities (Example 2). To reveal the functional significance of residues at positions 6, 7 and 8, truncation mutants of fowlicidin-1, namely fowlicidin-1(5-26), fowlicidin-1(6-26), fowlicidin-1(7-26) and fowlicidin-1(8-26) (Table VIII), were chemically synthesized using the standard solid-phase method by Bio-Synthesis (Lewisville, Tex.). All peptides were purified to >95% purity by reverse phase high-performance liquid chromatography (RP-HPLC). The mass and purity of each peptide were further confirmed by matrix-assisted laser desorption/ionization time-of-flight (MALDI-TOF) mass spectrometry using the Voyager DE-PRO instrument (Applied Biosystems, Foster City, Calif.) housed in the Recombinant DNA/Protein Core Facility of Oklahoma State University. Lyophilized peptides were reconstituted in 0.01% acetic acid. The peptide concentrations were measured by UV absorbance at 280 nm in the presence of 6 M guanidine hydrochloride, based on the extinction coefficients for aromatic tryptophan and tyrosine residues present in peptides.

TABLE VIII
Amino acid sequences of fowlicidin-1 and its analogs

| | | | |
|---|---|---|---|
| | | Molecular | SEQ ID |
| Peptide | Amino Acid Sequence | Weight | NO: |

| | | | |
|---|---|---|---|
| Fowlicidin-1(1-26) | RVKRVWPLVIRTVIAGYNLYRAIKKK | 3141.89 | 2 |
| Fowlicidin-1(5-26) | VWPLVIRTVIAGYNLYRAIKKK | 2602.21 | 6 |
| Fowlicidin-1(6-26) | WPLVIRTVIAGYNLYRAIKKK | 2503.08 | 9 |
| Fowlicidin-1(7-26) | PLVIRTVIAGYNLYRAIKKK | 2316.86 | 65 |
| Fowlicidin-1(8-26) | LVIRTVIAGYNLYRAIKKK | 2219.75 | 5 |

Bacterial culture and antibacterial testing. Gram-negative bacteria Salmonella enterica serovar Typhimurium DT104 ATCC 700408, and Klebsiella pneumoniae ATCC 13883), and Gram-positive bacteria (Listeria monocytogenes ATCC 19115, Staphylococcus aureus ATCC 25923, and S. aureus ATCC 43300) were purchased from either ATCC (Manassas, Va.) or MicroBiologics (St. Cloud, Minn.) and tested individually against fowlicidin-1 and its mutants. The minimum inhibitory concentrations (MICS) were determined by a standard broth microdilution assay as recommended by the Clinical and Laboratory Standards Institute. Briefly, overnight bacterial culture was subcultured in fresh trypticase soy broth with shaking at 250 rpm at 37° C. for 3 h to reach the mid-log phase. Bacteria were then washed twice in 10 mM sodium phosphate buffer, pH 7.4, and diluted to 5×10⁵ CFU/ml in Mueller Hinton broth (BBL, Cockeysville, Md.). Following dispensing 90 μl of bacteria into 96-well cell culture plates, 10 μl of peptides in serial two-fold dilutions were added in duplicate. The MIC value of each peptide was determined as the lowest peptide concentration that gave no visible bacterial growth after overnight incubation at 37° C.
Cytotoxicity assay. The toxicity was evaluated with human colonic epithelial Caco-2 cells (ATCC) by using alamarBlue dye (Biosource) as described (20,21). Briefly, cells were seeded into a 96-well plate at 5×10⁴ cells/well in Dulbecco's modified Eagle's medium (DMEM) containing 20% FBS. After overnight growth to 80-90% confluence at 37° C. and 5% CO₂, cells were washed once with DMEM, followed by addition of 90 μl of fresh DMEM with and without 10% FBS and 10 μl of each peptide in duplicate (to a final concentration of 100 or 200 mM) into each well. After incubation for 18 h, 10 μl of alamarBlue dye were added to each well and incubated for another 6 h. The fluorescence was measured with excitation at 545 nm and emission at 590 nm. Percent cell death (%) was calculated as [1−(F_{peptide}−F_{background})/(F_{acetic acid}−F_{background})]×100, where F_{peptide} is the fluorescence of cells exposed to peptides, F_{acetic acid} is the fluorescence of cells exposed to 0.01% acetic acid only, and F_{background} is the background fluorescence of 10% alamarBlue dye in DMEM without cells. Cytotoxicity (EC₅₀) of peptides will be defined as the peptide concentration that causes 50% cell death.

### Results and Discussion

The standard broth microdilution assay was used to test the activities of four fowlicidin analogs against three Gram-positive and two Gram-negative bacteria, including multidrug-resistant strains S. aureus ATCC 43300 and S. enterica serovar Typhimurium DT104 ATCC 700408. Interestingly, fowlicidin-1(5-26) and fowlicidin-1(6-26) showed identical MICs against all five bacterial strains tested (Table IX), clearly suggesting that valine at position 5 (V5) is dispensable for the antibacterial activity. Fowlicidin-1(7-26) and fowlicidin-1(8-26) also displayed similar antibacterial activities (Table IX), implying that P7 is not required for maintaining the antibacterial activity.

TABLE IX
MICs of fowlicidin-1 analogs against different bacterial strains*

| | MIC (μM) | | | | |
|---|---|---|---|---|---|
| | Fowl-1 | Fowl-1 | Fowl-1 | Fowl-1 | Fowl-1 |
| Bacteria | (1-26) | (5-26) | (6-26) | (7-26) | (8-26) |
| Staphyloccus aureus | 1 | 1 | 1 | 8 | 4 |
| ATCC 25923 | | | | | |
| Staphyloccus aureus | 1 | 1 | 1 | 4 | 2 |
| ATCC 43300 | | | | | |
| Listeria monocytogenes | 2 | 4 | 4 | 16 | 16 |
| ATCC 19115 | | | | | |
| Salmonella enterica | 2 | 4 | 4 | >16 | 16 |
| serovar Typhimurium | | | | | |
| DT104 ATCC 700408 | | | | | |
| Klebsiella pneumoniae | 2 | 2 | 2 | 16 | 16 |
| ATCC 13883 | | | | | |

*The MIC value for each peptide was repeated twice with similar results.

However, omission of W6 resulted in at least 4-fold reduction in bacteria-killing efficiency (compare the MIC values between fowlicidin-1(6-26) and fowlicidin-1(7-26) (Table IX), indicating the critical involvement of tryptophan in the antibacterial activity of fowlicidin-1. It is worthwhile to note that tryptophan is known to have a preference to be inserted into lipid bilayers at the membrane-water interface (3, 4). Because of such membrane-seeking ability, inclusion of tryptophan often renders peptides with higher affinity for membranes and more potency against bacteria (5, 6).

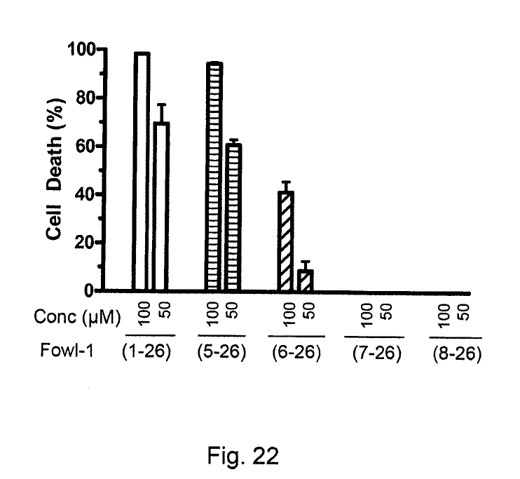

More desirably, fowlicidin-1(6-26) had substantially reduced toxicity to human colonic Caco-2 cells, relative to either the full-length peptide or fowlicidin-1(5-26) (FIG. 22), showing a further enhancement in safety and therapeutic window. Taken together, fowlicidin-1(6-26), retaining potent antibacterial activity with a significantly reduced cytotoxicity, is a short peptide analog with enhanced therapeutic potential as an antimicrobial drug candidate.

### References for Example 5

1. Xiao, Y., Cai, Y., Bommineni, Y. R., Fernando, S. C., Prakash, O., Gilliland, S. E., and Zhang, G. 2006. Identification and functional characterization of three chicken cathelicidins with potent antimicrobial activity. J Biol Chem 281:2858-2867.
2. Xiao, Y., Dai, H., Bommineni, Y. R., Soulages, J. L., Gong, Y. X., Prakash, O., and Zhang, G. 2006. Structure-activity relationships of fowlicidin-1, a cathelicidin antimicrobial peptide in chicken. FEBS J 273:2581-2593.
3. Yau, W. M., Wimley, W. C., Gawrisch, K., and White, S. H. 1998. The preference of tryptophan for membrane interfaces. Biochemistry 37:14713-14718.
4. Cook, G. A., Prakash, O., Zhang, K., Shank, L. P., Takeguchi, W. A., Robbins, A., Gong, Y. X., Iwamoto, T., Schultz, B. D., and Tomich, J. M. 2004. Activity and structural comparisons of solution associating and monomeric channel-forming peptides derived from the glycine receptor m2 segment. Biophys J 86:1424-1435.
5. Deslouches, B., Phadke, S. M., Lazarevic, V., Cascio, M., Islam, K., Montelaro, R. C., and Mietzner, T. A. 2005. De novo generation of cationic antimicrobial peptides: influence of length and tryptophan substitution on antimicrobial activity. Antimicrob Agents Chemother 49:316-322.
6. Subbalakshmi, C., Bikshapathy, E., Sitaram, N., and Nagaraj, R. 2000. Antibacterial and hemolytic activities of single tryptophan analogs of indolicidin. Biochem Biophys Res Commun 274:714-716.

### Example 6

### Rational Design of Fowlicidin Analogs

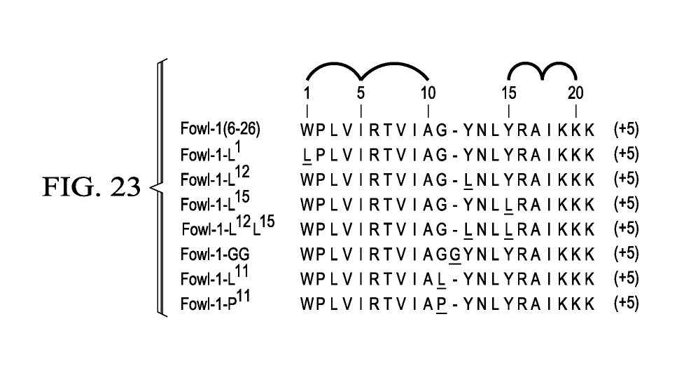

Further confirmation the functional significance of tryptophan (W1) of fowlicidin-1(6-26) (Table IX of Example 5), this residue is mutated to leucine (FIG. 23). Such a W1L mutation (Fowl-1-L1) will only cause minimal alterations in the physiochemical properties of the parent peptide. It is also interesting to note the presence of two lipophilic tyrosines, which are known to be important in membrane interactions, in the major antibacterial domain. Both tyrosines are mutated sequentially or simultaneously to leucines (FIG. 23).

A scan of over 150 helical antimicrobial peptides revealed that glycine is frequently found near the center and acts as a hinge to increase flexibility (51). Presence or introduction of such a hinge in the helix has been shown in many cases to be desirable, attenuating the toxicity of peptides to host cells while maintaining comparable antimicrobial potency with the peptides with no hinge (51, 54, 93). To investigate the influence of structural flexibility on the functions of fowlicidin-1, several analogs are designed (FIG. 23). An extra glycine (fowl-1-GG) is added to the kink region to further enhance the flexibility of two short flanking α-helical segments. A replacement of G¹¹ with proline (fowl-1-P¹¹) is also designed to increase the bend but minimize the flexibility of the two short helices. Conversely, fowl-1-L¹¹ is synthesized with a helix-stabilizing residue, leucine, being substituted for G¹¹. Such a variant minimizes both the bend and flexibility between the two short helices without significantly altering other structural characteristics.

Similarly, a series of fowl-2(1-18) and fowl-2(15-31) analogs is also designed and compared for antibacterial, cytotoxic, and endotoxin-neutralizing activities. Various deletion variants are also made to probe the minimal sequence required for antimicrobial activity of fowlicidins. C-terminal amidation of these short fragments is performed as well.

Increasing the kink or flexibility of α-helix by introducing proline or an additional glycine is results in retention of antibacterial and endotoxin-neutralizing activity with a reduced cytotoxicity, whereas a reduction in flexibility by replacing glycine with leucine increases toxicity. C-terminal amidation of these short fragments enhances the stability and antibacterial activity as well.

TABLE X

Listing of peptides cited herein and the corresponding
SEQ ID NOS.

| | | |
|---|---|---|
| | Sequence and/or | SEQ ID |
| Peptide | Location in Specification | NO: |

| | | |
|---|---|---|
| Fowlicidin-1 (-2-26) | PVRVKRVWPLVIRTVIAGYNLYRAIKKK; | 1 |
| | FIG. 10 | |
| Fowlicidin-1 | RVKRVWPLVIRTVIAGYNLYRAIKKK; | 2 |
| | FIGS. 11, 17B; Table IV; Table VIII | |
| Fowl-1(1-15) | RVKRVWPLVIRTVIA; Table IV | 3 |
| Fowl-1(1-23) | RVKRVWPLVIRTVIAGYNLYRAI; Table IV | 4 |
| Fowl-1(8-26) | LVIRTVIAGYNLYRAIKKK; Table IV; Table | 5 |
| | VIII | |
| Fowl-1(5-26) | VWPLVIRTVIAGYNLYRAIKKK; Table IV | 6 |
| Fowl-1-L¹⁶ | RVKRVWPLVIRTVIALYNLYRAIKKK; Table | 7 |
| | IV | |
| Fowl-1- | RVKRVWKLVIRLVKALYKLYRAIKKK; Table | 8 |
| K⁷L¹²K14L¹⁶K¹⁸ | IV | |
| Fowlicidin-1 (6-26) | WPLVIRTVIAGYNLYRAIKKK; | 9 |
| | FIG. 23; Table VIII | |
| Fowl-1-L¹ | LPLVIRTVIAGYNLYRAIKKK; | 10 |
| | FIG. 23 | |
| Fowl-1-L¹² | WPLVIRTVIAGLNLYRAIKKK; | 11 |
| | FIG. 23 | |
| Fowl-1-L¹⁵ | WPLVIRTVIAGYNLLRAIKKK; | 12 |
| | FIG. 23 | |
| Fowl-1-L¹²L¹⁵ | WPLVIRTVIAGLNLLRAIKKK; | 13 |
| | FIG. 23 | |
| Fowl-1-GG | WPLVIRTVIAGGYNLYRAIKKK; | 14 |
| | FIG. 23 | |
| Fowl-1-L¹¹ | WPLVIRTVIALYNLYRAIKKK; | 15 |
| | FIG. 23 | |
| Fowl-1-P¹¹ | WPLVIRTVIAPYNLYRAIKKK; | 16 |
| | FIG. 23 | |
| Fowlicidin-2 | LVQRGRFGRFLRKIRRFRPKVTITIQGSARF | 17 |
| | Table VI | |
| Fowlicidin-3 | KRFWPLVPVAINTVAAGINLYKAIRRK; FIG. | 18 |
| | 17B | |
| Fowlicidin-3 (4-27) | WPLVPVAINTVAAGINLYKAIKKK | 66 |
| Fowlicidin-1 with | MLSCWVLLLALLGGACALPAPLGYSQALAQ | 19 |
| signal peptide and | AVDSYNQRPEVQNAFRLLSADPEPGPNVQLS | |
| prosequence | SLHNLNFTIMETRCQARSGAQLDSCEFKEDG | |
| | LVKDCAAPVVLQGGRAVLDVTCVDSMADPV | |
| | RVKRVWPLVIRTVIAGYNLYRAIKKK | |
| | FIG. 1 | |
| Fowlicidin-2 with | MLSCWVLLLALLGGACALPAPLGYSQALAQ | 20 |
| signal peptide and | AVDSYNQRPEVQNAFRLLSADPEPGPGVDLS | |
| prosequence | TLRALNFTIMETECTPSARLPVDDCDFKENGV | |
| | IRDCSGPVSVLQDTPEINLRCRDASSDPVLVQ | |
| | RGRFGRFLRKIRRFRPKVTITIQGSARFG | |
| | FIG. 1 | |
| Fowlicidin-3 with | MLSCWVLLLALLGGACALPAPLGYSQALAQ | 21 |
| signal peptide and | AVDSYNQRPEVQNAFRLLSADPEPGPNVQLS | |
| prosequence | SLHNLNFTIMETRCQARSGAQLDSCEFKEDG | |
| | LVKDCAAPVVLQGGRAVLDVTCVDSMADPV | |
| | RVKRFWPLVPVAINTVAAGINLYKAIRRK | |
| | FIG. 1 | |
| pNGP with signal | MAGAWRALVLVAGLAAVVSVAQRSLSYEEII | 22 |
| peptide and | NKALWFFNQGRPGQRLFRLLVAMPPPNLNFT | |
| prosequence | TNIPLNFRIKETVCFSTRFHLHRQPRKCAFREG | |
| | GEERNCNGTFFMLPQFRLLSFQCTEDPDRERE | |
| | LNRQIPRVRRSAVSSDVAPPETDISKLPPAAR | |
| | DLYERTKYDIINNILRNF | |
| | FIG. 1 | |
| bNGP with signal | MAGAWKALVLVAGLAAVACVAQRGLSYEEI | 23 |
| peptide and | VTQALKFFNQGRRGQRIFGLLESTPPPPDLNS | |
| prosequence | TTIPLNFRIKETVCFLLWYRRRPRQCPFREGG | |
| | EERNCTGSFFMLRQLRLLSLNCVPDRELEPEP | |
| | RRRERRSAGSAGEDPPELDSSNLPPAVRDMY | |
| | ERAKYDIISNILRNF | |
| | FIG. 1 | |
| rNGP with signal | MARLWKTFMLVVALAVVACEAHRRLRYEDI | 24 |
| peptide and | VNRAIEAYNRGQRGRPLFRLLSATPPPGQNPT | |
| prosequence | SNVPLEFRIKETVCISTTERRLENCDFREGGEE | |
| | RNCTGEFSRRQWSTSLTLTCDRDCRREVSQV | |
| | ATFSDNKSDDSEKDKLEGLPPHAKNIYENAK | |
| | YDIISNILHNF | |
| | FIG. 1 | |
| mNGP with signal | MAGLWKTFVLVVALAVVSCEALRQLRYEEI | 25 |
| peptide and | VDRAIEAYNQGRQGRPLFRLLSATPPSSQNPA | |
| prosequence | TNIPLQFRIKETECTSTQERQPKDCDFLENGEE | |
| | RNCTGKFFRRRQSTSLTLTCDRDCSREDTQET | |
| | SFNDKQDVSEKEKFEDVPPHIRNIYEDAKYDI | |
| | IGNILKNF | |
| | FIG. 1 | |
| P15 with signal | MAGVWKVLVVLVGLAVVACAIPHRRLRYEE | 26 |
| peptide and | VVAQALQFYNEGQQGQPLFRLLEATPPPSLN | |
| prosequence | SKSRIPLNFRIKETVCIFTLDRQPGNCAFREGG | |
| | EERICRGAFVRRRRVRALTLRCDRDQRRQPE | |
| | FPRVTRPAGPTA | |
| | FIG. 1 | |
| LL37 with signal | MKTQRNGHSLGRWSLVLLLLGLVMPLAIIAQ | 27 |
| peptide and | VLSYKEAVLRAIDGINQRSSDANLYRLLDLDP | |
| prosequence | RPTMDGDPDTPKPVSFTVKETVCPRTTQQSPE | |
| | DCDFKKDGLVKRCMGTVTLNQARGSFDISC | |
| | DKDNKRFALLGDFFRKSKEKIGKEFKRIVQRI | |
| | KDFLRNLVPRTES | |
| | FIG. 1 | |
| CRAMP with signal | MQFQRDVPSLWLWRSLSLLLLLGLGFSQTPS | 28 |
| peptide and | YRDAVLRAVDDFNQQSLDTNLYRLLDLDPEP | |
| prosequence | QGDEDPDTPKSVRFRVKETVCGKAERQLPEQ | |
| | CAFKEQGVVKQCMGAVTLNPAADSFDISCNE | |
| | PGAQPFRFKKISRLAGLLRKGGEKIGEKLKKI | |
| | GQKIKNFFQKLVPQPEQ | |
| | FIG. 1 | |
| Protegrin-1 with | METQRASLCLGRWSLWLLLLALVVPSASAQ | 29 |
| signal peptide and | ALSYREAVLRAVDRLNEQSSEANLYRLLELD | |
| prosequence | QPPKADEDPGTPKPVSFTVKETVCPRPTRQPP | |
| | ELCDFKENGRVKQCVGTVTLDQIKDPLDITC | |
| | NEVQGVRGGRLCYCRRRFCVCVGRG | |
| | FIG. 1 | |
| Indolicidin with | MQTQRASLSLGRWSLWLLLLGLVVPSASAQ | 30 |
| signal peptide and | ALSYREAVLRAVDQLNELSSEANLYRLLELD | |
| prosequence | PPPKDNEDLGTRKPVSFTVKETVCPRTIQQPA | |
| | EQCDFKEKGRVKQCVGTVTLDPSNDQFDLN | |
| | CNELQSVILPWKWPWWPWRRG | |
| | FIG. 1 | |
| Melittin | GIGAVLKVLTTGLPALISWIKRKRQQ | 31 |
| | FIG. 10 | |
| Cecropin A-1 | GWLKKIGKKIERVGQHTRDATIQGLGIAQQA | 32 |
| | ANVAATAR | |
| | FIG. 10 | |
| Cecropin P-1 | SWLSKTAKKLENSAKKRISEGIAIAIQGGPR | 33 |
| | FIG. 10 | |
| CRAMP | GLLRKGGEKIGEKLKKIGQKIKNFFQKLVPQP | 34 |
| | EQ | |
| | FIG. 10 | |
| CAP 18 | GLRKRLRKFRNKIKEKLKKIGQKIQGFVPKLA | 67 |
| | PRTDY; FIG. 10 | |
| BMAP34 | GLFRRLRDSIRRGQQKILEKARRIGERIKDIFRG | 35 |
| | FIG. 10 | |
| SMAP34 | GLFGRLRDSLQRGGQKILEKAERIGDRIKDIF | 36 |
| | RG | |
| | FIG. 10 | |
| PMAP37 | GLLSRLRDFLSDRGRRLGEKIERIGQKIKDLSE | 37 |
| | FFQS | |
| | FIG. 10 | |
| BMAP28 | GGLRSLGRKILRAWKKYGPIIVPIIRIG; | 38 |
| | FIG. 10 | |
| SMAP29 | RGLRRLGRKIAHGVKKYGPTVLRIIRIAG; | 39 |
| | FIG. 10 | |
| Fowl-2(1-31) | LVQRGRFGRFLRKIRRFRPKVTITIQGSARF; | 40 |
| | Table VI | |
| Fowl-2(1-14) | LVQRGRFGRFLRKI; Table VI | 41 |
| Fowl-2(1-15) | LVQRGRFGRFLRKIR; Table VI | 42 |
| Fowl-2(1-18) | LVQRGRFGRFLRKIRRFR; Table VI | 43 |
| Fowl-2(15-31) | RRFRPKVTITIQGSARF; Table VI | 44 |
| Fowl-2(19-31) | PKVTITIQGSARF; Table VI | 45 |
| Fowlicidin 1(7-26) | PLVIRTVIAGYNLYRAIKKK; Table VIII | 65 |

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1-19. (canceled)

20. An isolated bactericidal peptide with an amino acid sequence consisting of VWPLV IRTVIAGYNLYRAIKKK (SEQ ID NO: 6) or a truncated bactericidal portion thereof with an amino acid sequence consisting of WPLVIRTVIAGYNLYRAIKKK (SEQ ID NO: 9), or derivatives thereof with conservative amino acid substitutions,
wherein a carboxy terminus of said isolated bactericidal peptide or said truncated bactericidal portion thereof is amidated.

21. A method of killing or damaging a bacterium, comprising the step of
contacting said bacterium with a bactericidal peptide with an amino acid sequence consisting of VWPLVIRTVIAGYNLYRAIKKK (SEQ ID NO: 6) or a truncated bactericidal portion thereof with an amino acid sequence consisting of WPLVIRTVIAGYNLYRAIKKK (SEQ ID NO: 9), or a derivative thereof with conservative amino acid substitutions,
wherein a carboxy terminus of said bactericidal peptide or said truncated bactericidal portion thereof is amidated, and wherein said bactericidal peptide or said truncated bactericidal portion thereof is provided in a quantity sufficient to kill or damage said bacterium.

22. The method of claim 21, wherein said bacterium is an antibiotic resistant bacterium.

23. A method of treating a bacterial infection in a patient in need thereof, comprising the step of
administering to said patient in a quantity sufficient to treat said bacterial infection, a bactericidal peptide with an amino acid sequence consisting of VWPLVIRTVIAGYNLYRAIKKK (SEQ ID NO: 6) or a truncated bactericidal portion thereof with an amino acid sequence consisting of WPLVIRTVIAGYNLYRAIKKK (SEQ ID NO: 9), or a derivative thereof with conservative amino acid substitutions,
wherein a carboxy terminus of said bactericidal peptide or said truncated bactericidal portion thereof is amidated.

24. The method of claim 23, wherein said bacterial infection is caused by an antibiotic resistant bacterium.


SEQUENCE LISTING (71 sequences)

SEQ ID NO: 1 - protein, 28 aa, Artificial
  truncated version of fowlicidin-1
PVRVKRVWPLVIRTVIAGYNLYRAIKKK

SEQ ID NO: 2 - protein, 26 aa, Artificial
  truncated fowlicidin-1
RVKRVWPLVIRTVIAGYNLYRAIKKK

SEQ ID NO: 3 - protein, 15 aa, Artificial
  truncated fowlicidin-1
RVKRVWPLVIRTVIA

SEQ ID NO: 4 - protein, 23 aa, Artificial
  truncated fowlicidin-1
RVKRVWPLVIRTVIAGYNLYRAI

SEQ ID NO: 5 - protein, 19 aa, Artificial
  truncated fowlicidin-1
LVIRTVIAGYNLYRAIKKK

SEQ ID NO: 6 - protein, 22 aa, Artificial
  truncated fowlicidin-1
VWPLVIRTVIAGYNLYRAIKKK

SEQ ID NO: 7 - protein, 26 aa, Artificial
  truncated fowlicidin-1
RVKRVWPLVIRTVIALYNLYRAIKKK

SEQ ID NO: 8 - protein, 26 aa, Artificial
  truncated fowlicidin-1
RVKRVWKLVIRLVKALYKLYRAIKKK

SEQ ID NO: 9 - protein, 21 aa, Artificial
  truncated fowlicidin-1
WPLVIRTVIAGYNLYRAIKKK

SEQ ID NO: 10 - protein, 21 aa, Artificial
  truncated fowlicidin-1 variant
LPLVIRTVIAGYNLYRAIKKK

SEQ ID NO: 11 - protein, 21 aa, Artificial
  truncated fowlicidin-1 variant
WPLVIRTVIAGLNLYRAIKKK

SEQ ID NO: 12 - protein, 21 aa, Artificial
  truncated fowlicidin-1 variant
WPLVIRTVIAGYNLLRAIKKK

SEQ ID NO: 13 - protein, 21 aa, Artificial
  truncated fowlicidin-1 variant
WPLVIRTVIAGLNLLRAIKKK

SEQ ID NO: 14 - protein, 22 aa, Artificial
  truncated fowlicidin-1 variant
WPLVIRTVIAGGYNLYRAIKKK

SEQ ID NO: 15 - protein, 21 aa, Artificial
  truncated fowlicidin-1 variant
WPLVIRTVIALYNLYRAIKKK

SEQ ID NO: 16 - protein, 21 aa, Artificial
  truncated fowlicidin-1 variant
WPLVIRTVIAPYNLYRAIKKK

SEQ ID NO: 17 - protein, 31 aa, Artificial
  truncated version of fowlicidin-2
LVQRGRFGRFLRKIRRFRPKVTITIQGSARF

SEQ ID NO: 18 - protein, 27 aa, Artificial
  truncated version of fowlicidiin-3
KRFWPLVPVAINTVAAGINLYKAIRRK

SEQ ID NO: 19 - protein, 148 aa, Gallus gallus
MLSCWVLLLALLGGACALPAPLGYSQALAQAVDSYNQRPEVQNAFRLLSADPEPGPNVQLSSLHNLNFTIMETRCQARSGAQLDSCEFKEDGLVKDCAAPVVLQGGRAVLDVTCVDSMADPVRVKRVWPLVIRTVIAGYNLYRAIKKK

SEQ ID NO: 20 - protein, 154 aa, Chicken
MLSCWVLLLALLGGACALPAPLGYSQALAQAVDSYNQRPEVQNAFRLLSADPEPGPGVDLSTLRALNFTIMETECTPSARLPVDDCDFKENGVIRDCSGPVSVLQDTPEINLRCRDASSDPVLVQRGRFGRFLRKIRRFRPKVTITIQGSARFG

SEQ ID NO: 21 - protein, 151 aa, Gallus gallus
MLSCWVLLLALLGGACALPAPLGYSQALAQAVDSYNQRPEVQNAFRLLSADPEPGPNVQLSSLHNLNFTIMETRCQARSGAQLDSCEFKEDGLVKDCAAPVVLQGGRAVLDVTCVDSMADPVRVKRFWPLVPVAINTVAAGINLYKAIRRK

SEQ ID NO: 22 - protein, 177 aa, Homo sapiens
MAGAWRALVLVAGLAAVVSVAQRSLSYEEIINKALWFFNQGRPGQRLFRLLVAMPPPNLNFTTNIPLNFRIKETVCFSTRFHLHRQPRKCAFREGGEERNCNGTFFMLPQFRLLSFQCTEDPDRERELNRQIPRVRRSAVSSDVAPPETDISKLPPAARDLYERTKYDIINNILRNF

SEQ ID NO: 23 - protein, 172 aa, Bos taurus
MAGAWKALVLVAGLAAVACVAQRGLSYEEIVTQALKFFNQGRRGQRIFGLLESTPPPPDLNSTTIPLNFRIKETVCFLLWYRRRPRQCPFREGGEERNCTGSFFMLRQLRLLSLNCVPDRELEPEPRRRERRSAGSAGEDPPELDSSNLPPAVRDMYERAKYDIISNILRNF

SEQ ID NO: 24 - protein, 168 aa, Rattus norvegicus
MARLWKTFMLVVALAVVACEAHRRLRYEDIVNRAIEAYNRGQRGRPLFRLLSATPPPGQNPTSNVPLEFRIKETVCISTTERRLENCDFREGGEERNCTGEFSRRQWSTSLTLTCDRDCRREVSQVATFSDNKSDDSEKDKLEGLPPHAKNIYENAKYDIISNILHNF

SEQ ID NO: 25 - protein, 167 aa, Bos taurus
MAGLWKTFVLVVALAVVSCEALRQLRYEEIVDRAIEAYNQGRQGRPLFRLLSATPPSSQNPATNIPLQFRIKETECTSTQERQPKDCDFLENGEERNCTGKFFRRRQSTSLTLTCDRDCSREDTQETSFNDKQDVSEKEKFEDVPPHIRNIYEDAKYDIIGNILKNF

SEQ ID NO: 26 - protein, 137 aa, Oryctolagus cuniculus
MAGVWKVLVVLVGLAVVACAIPHRRLRYEEVVAQALQFYNEGQQGQPLFRLLEATPPPSLNSKSRIPLNFRIKETVCIFTLDRQPGNCAFREGGEERICRGAFVRRRRVRALTLRCDRDQRRQPEFPRVTRPAGPTA

SEQ ID NO: 27 - protein, 170 aa, Homo sapiens
MKTQRNGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 28 - protein, 173 aa, Mus musculus
MQFQRDVPSLWLWRSLSLLLLLGLGFSQTPSYRDAVLRAVDDFNQQSLDTNLYRLLDLDPEPQGDEDPDTPKSVRFRVKETVCGKAERQLPEQCAFKEQGVVKQCMGAVTLNPAADSFDISCNEPGAQPFRFKKISRLAGLLRKGGEKIGEKLKKIGQKIKNFFQKLVPQPEQ

SEQ ID NO: 29 - protein, 149 aa, Sus scrofa
METQRASLCLGRWSLWLLLLALVVPSASAQALSYREAVLRAVDRLNEQSSEANLYRLLELDQPPKADEDPGTPKPVSFTVKETVCPRPTRQPPELCDFKENGRVKQCVGTVTLDQIKDPLDITCNEVQGVRGGRLCYCRRRFCVCVGRG

SEQ ID NO: 30 - protein, 144 aa, Bos taurus
MQTQRASLSLGRWSLWLLLLGLVVPSASAQALSYREAVLRAVDQLNELSSEANLYRLLELDPPPKDNEDLGTRKPVSFTVKETVCPRTIQQPAEQCDFKEKGRVKQCVGTVTLDPSNDQFDLNCNELQSVILPWKWPWWPWRRG

SEQ ID NO: 31 - protein, 26 aa, Apis mellifera
GIGAVLKVLTTGLPALISWIKRKRQQ

SEQ ID NO: 32 - protein, 39 aa, Drosophila melanogaster
GWLKKIGKKIERVGQHTRDATIQGLGIAQQAANVAATAR

SEQ ID NO: 33 - protein, 31 aa, Sus scrofa
SWLSKTAKKLENSAKKRISEGIAIAIQGGPR

SEQ ID NO: 34 - protein, 34 aa, Mus musculus
GLLRKGGEKIGEKLKKIGQKIKNFFQKLVPQPEQ

SEQ ID NO: 35 - protein, 34 aa, Bos taurus
GLFRRLRDSIRRGQQKILEKARRIGERIKDIFRG

SEQ ID NO: 36 - protein, 34 aa, Ovis aries
GLFGRLRDSLQRGGQKILEKAERIGDRIKDIFRG

SEQ ID NO: 37 - protein, 37 aa, Sus scrofa
GLLSRLRDFLSDRGRRLGEKIERIGQKIKDLSEFFQS

SEQ ID NO: 38 - protein, 28 aa, Bos taurus
GGLRSLGRKILRAWKKYGPIIVPIIRIG

SEQ ID NO: 39 - protein, 29 aa, Ovis aries
RGLRRLGRKIAHGVKKYGPTVLRIIRIAG

SEQ ID NO: 40 - protein, 31 aa, Artificial; identical to SEQ ID NO: 17
  truncated version of fowlicidin-2

SEQ ID NO: 41 - protein, 14 aa, Artificial
  truncated version of fowlicidin-2
LVQRGRFGRFLRKI

SEQ ID NO: 42 - protein, 15 aa, Artificial
  truncated version of fowlicidin-2
LVQRGRFGRFLRKIR

SEQ ID NO: 43 - protein, 18 aa, Artificial
  truncated version of fowlicidin-2
LVQRGRFGRFLRKIRRFR

SEQ ID NO: 44 - protein, 17 aa, Artificial
  truncated version of fowlicidin-2
RRFRPKVTITIQGSARF

SEQ ID NO: 45 - protein, 13 aa, Artificial
  truncated version of fowlicidin-2
PKVTITIQGSARF

SEQ ID NO: 46 - DNA, 52 bp, Artificial
  synthetic oligonucleotide primer
caagggaggacgctgtctgtcgaaggtaaggaacggacgagagaagggagag

SEQ ID NO: 47 - DNA, 53 bp, Artificial
  synthetic oligonucleotide primer
ctctcccttctcgaatcgtaaccgttcgtacgagaatcgctgtcctctccttg

SEQ ID NO: 48 - DNA, 23 bp, Artificial
  synthetic oligonucleotide primer
gtttccgcattgcccaacttcag

SEQ ID NO: 49 - DNA, 22 bp, Artificial
  synthetic oligonucleotide primer
ggaacagtgctaacagtggctc

SEQ ID NO: 50 - DNA, 22 bp, Artificial
  synthetic oligonucleotide primer
gctgtggactcctacaaccaac

SEQ ID NO: 51 - DNA, 22 bp, Artificial
  synthetic oligonucleotide primer
ttgaggttgtgcagggagctga

SEQ ID NO: 52 - DNA, 21 bp, Artificial
  synthetic oligonucleotide primer
cgctggtcatcaggactgtga

SEQ ID NO: 53 - DNA, 19 bp, Artificial
  synthetic oligonucleotide primer
ccatcgtgtctccattcta

SEQ ID NO: 54 - DNA, 21 bp, Artificial
  synthetic oligonucleotide primer
caccgtgttgatggccactgg

SEQ ID NO: 55 - DNA, 21 bp, Artificial
  synthetic oligonucleotide primer
tgaggccaccgagtgtcacct

SEQ ID NO: 56 - protein, 28 aa, Ovis aries
RGLRRLGRKIAHGVKKYGPTVLRIIRIA

SEQ ID NO: 57 - DNA, 22 bp, Artificial
  synthetic oligonucleotide primer
agaatctatacctgtcctgtgt

SEQ ID NO: 58 - DNA, 20 bp, Artificial
  synthetic ologonucleotide primer
tgtgctctgcttgtgaggtg

SEQ ID NO: 59 - DNA, 20 bp, Artificial
  synthetic oligonucleotide primer
acaagaggatcaccagcagc

SEQ ID NO: 60 - DNA, 21 bp, Artificial
  synthetic oligonucleotide primer
ctgaagaccttagggcagatg

SEQ ID NO: 61 - DNA, 20 bp, Artificial
  synthetic oligonucleotide primer
cacgccaattcatcgttgac

SEQ ID NO: 62 - DNA, 20 bp, Artificial
  synthetic oligonucleotide primer
cattcagttccaggtcagtg

SEQ ID NO: 63 - DNA, 20 bp, Artificial
  synthetic oligonucleotifde primer
ggagattactgctctggctc

SEQ ID NO: 64 - DNA, 20 bp, Artificial
  synthetic oligonucleotide primer
ctcctgcttgctgatccaca

SEQ ID NO: 65 - protein, 20 aa, Artificial
  truncated version of fowlicidin-1
PLVIRTVIAGYNLYRAIKKK

SEQ ID NO: 66 - protein, 24 aa, Artificial
  truncated version of fowlicidin-3
WPLVPVAINTVAAGINLYKAIKKK

SEQ ID NO: 67 - protein, 37 aa, Oryctolagus cuniculus
GLRKRLRKFRNKIKEKLKKIGQKIQGFVPKLAPRTDY

SEQ ID NO: 68 - protein, 28 aa, Artificial
  Synthetic consensus peptide sequence
KRFXPLVPVAIXTVXAXXXNLXXAIXXX

SEQ ID NO: 69 - protein, 25 aa, Artificial
  Synthetic consensus peptide sequence
XPLVPVAIXTVXAXXXNLXXAIXXX

SEQ ID NO: 70 - protein, 25 aa, Artificial
  Synthetic consensus peptide sequence
KRFXPLVIXTVXAXXXNLXXAIXXX

SEQ ID NO: 71 - protein, 22 aa, Artificial
  Synthetic consensus peptide sequence
XPLVIXTVXAXXXNLXXAIXXX